(19) United States
(12) Patent Application Publication
(10) Pub. No.: US 2024/0156885 A1
(43) Pub. Date: May 16, 2024

(54) VAGINAL MICROBIOTA-ASSOCIATED METHODS, COMPOSITIONS, AND DEVICES

(71) Applicant: Freya Biosciences ApS, Copenhagen Ø (DK)

(72) Inventors: Colleen Denise ACOSTA, Los Gatos, CA (US); Johan E.T. VAN HYLCKAMA, Belmont, MA (US); Thomas Gundelund RASMUSSEN, Wellesley, MA (US); Elleke Fenna BOSMA, Copenhagen (DK); Brynjulf MORTENSEN, Virum (DK)

(21) Appl. No.: 18/549,163

(22) PCT Filed: Mar. 3, 2022
(86) PCT No.: PCT/IB2022/000099

§ 371 Date: Sep. 5, 2023

Related U.S. Application Data
(60) Provisional application No. 63/156,328, filed on Mar. 3, 2021.

(51) Int. Cl. A61K 35/747; A61K 9/00; A61K 9/02; A61K 9/70; A61K 47/32; A61K 47/36; A61K 47/38; A61P 15/02

(52) U.S. Cl. CPC A61K 35/747; A61K 9/0034; A61K 9/02; A61K 9/7007; A61K 47/32; A61K 47/36; A61K 47/38; A61P 15/02

(57) ABSTRACT

The invention relates to a pharmaceutical composition for use in treating inflammation in the female genitourinary tract of a human subject, wherein the female subject exhibits a dysbiotic microbiota in the genitourinary tract. The pharmaceutical composition comprises a substantially complete vaginal microbiota preparation, wherein the preparation (i) comprises one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the preparation; and (ii) comprises less than 5% of Gardnerella spp., Atopobium spp., and Prevotella spp.; wherein the pharmaceutical composition comprises a pharmaceutically acceptable carrier or diluent. The invention further relates to a method of preparing said composition, and devices comprising and/or using the same.

### RELATED APPLICATIONS

This application claims the benefit of U.S. Provisional Application No. 63/156,328, filed on Mar. 3, 2021, which is incorporated herein by reference in its entirety.

### BACKGROUND OF THE INVENTION

The vagina is a fibromuscular tubular tract leading from the uterus to the exterior of the body in females. A healthy vagina is colonized by a mutually symbiotic flora of microorganisms, in particular of the genus Lactobacillus, that protects its host from vaginal infections. The acidity of a healthy vagina of a woman of child-bearing age (pH about 3.8-4.5) is thought to be due to the degradation of secreted glycogen/glucose to lactic acid and acetate by lactobacilli. Acidic conditions are unfavorable for the growth of many pathogenic microorganisms and pathobionts, including bacteria, protozoa and viruses. However, an imbalance in the vaginal microbiota may result in overgrowth of pathogenic microorganisms and pathobionts, resulting in dysbiosis, inflammation and/or infections.

Multiple kinds of vaginal infections (vaginitis) exist, depending on the pathogenic microorganism involved such as, bacterial vaginosis, vaginal candidiasis and trichomoniasis and combinations thereof.

Lactobacillus-containing products (comprising, e.g., Lactobacillus acidophilus, Lactobacillus rhamnosus, or Lactobacillus gasseri) for intravaginal or oral use have been available for many years. These products include vaginal suppositories containing lyophilized Lactobacillus acidophilus or other Lactobacillus species (e.g., Lactobacillus rhamnosus, or Lactobacillus gasseri) of human origin as well as various nutritional supplements. These products have been largely non-efficacious due to the products' inability to colonize the vagina with the exogenous lactobacilli. This might be due to poor quality or the use of ecologically unsuitable strains.

The colonization and establishment of isolated bacteria, such as single strains, in the vaginal microbial niche is difficult to predict from in vitro experiments, e.g., it is common for different bacterial species in the vaginal microbial niche to interact, and these interactions are difficult to recreate in the laboratory. It is well established and broadly accepted that different women can be colonized by different compositions of species/strains of lactobacilli and that this composition of lactobacilli changes over time. Thus, even if bacterial species display promising in vitro results, it might not colonize or engraft in vivo and provide the desired therapeutic effect, or the effect is transient as the strains(s) delivered are not able to collectively adapt to provide a maintained colonization over time. For example, a specified value for adherence to the vaginal mucosa (vaginal epithelial cells—VEC), ability to produce anti-microbial/anti-pathogenic substances, such as, e.g., hydrogen peroxide and/or bacteriocins, ability to produce lactic acid for acidification, and fast doubling time, in culture are often measured in vitro, though these characteristics have not routinely led to successful predictions of the ability of specific strains to engraft in the vaginal niche (their ability to colonize the vagina) in vivo.

Thus, a need exists for the development of additional therapeutic compositions for vaginal diseases and adverse health conditions in women.

### SUMMARY OF THE INVENTION

The present invention addresses this unmet need by providing compositions, substantially complete vaginal microbiota preparations, methods of producing the same and therapeutic methods. The compositions and substantially complete vaginal microbiota preparations are effective in the treatment of diseases, symptoms and/or disorders of the female urogenital tract, including inflammation of the urogenital tract, well-tolerated, without adverse effects, and/or effectively colonize the vaginal niche.

It shall be understood that topics which are discussed for, e.g., the first aspect will also apply for the second aspect, and any other aspects unless the technical context requires otherwise or unless the discussed topic clearly pertains to one aspect only. For example, in the context of the first aspect it is mentioned that a substantially complete vaginal microbiota preparation comprises four bacterial species from the genus Lactobacillus, and that the one to four bacterial species comprise about 80-99.9% of the preparation, wherein the one to four Lactobacillus comprise Lactobacillus crispatus, Lactobacillus iners, Lactobacillus jensenii and Lactobacillus gasseri. The preparation may comprise about 80-99.9% Lactobacillus crispatus. The preparation may comprise about 80-99.9% Lactobacillus iners. The preparation may comprise about 80-99.9% Lactobacillus jensenii. The preparation may comprise about 80-99.9% Lactobacillus gasseri. The preparation may comprise about 80-99.9% of one, two, three, or four of Lactobacillus crispatus, Lactobacillus iners, Lactobacillus jensenii, and Lactobacillus gasseri.

If in the context of the second aspect or another aspect, a substantially complete vaginal microbiota preparation is mentioned, this also means that the same Lactobacillus species, relative quantities and combinations of the first aspect are intended that can be used in the substantially complete vaginal microbiota preparation of the second or any other aspect even if this is not explicitly mentioned for the second aspect.

The disclosure for the first, second, and any other aspect shall thus be assessed in combination as reflecting embodiments of the same overarching principles, and not as unrelated, separate chapters.

Described herein, in a first aspect, are pharmaceutical compositions for use in treating inflammation in the female genitourinary tract of a human subject, wherein the female subject exhibits a dysbiotic microbiota in the genitourinary tract, said method comprising administering to the subject an effective amount of the pharmaceutical composition, wherein the pharmaceutical composition comprises a substantially complete vaginal microbiota preparation, wherein the preparation (i) comprises one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the preparation; and (ii) comprises less than 5% of Gardnerella spp., Atopobium spp., and Prevotella spp.; wherein the pharmaceutical composition comprises a pharmaceutically acceptable carrier or diluent.

In a second aspect, provided herein are pharmaceutical compositions comprising a substantially complete vaginal microbiota preparation, wherein the preparation (i) comprises one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the preparation; and (ii) comprises <5% of Gardnerella spp., Atopobium spp., and Prevotella spp.; and comprises a pharmaceutically acceptable carrier or diluent, optionally for use in treating inflammation in the female genitourinary tract of a human subject, wherein the female subject exhibits a dysbiotic microbiota in the genitourinary tract.

In a third aspect, provided herein are substantially complete vaginal microbiota preparations or pharmaceutical compositions comprising the same, wherein said preparation (i) comprises one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the preparation; and (ii) comprises less than 5% of Gardnerella spp., Atopobium spp., and Prevotella spp.; wherein the pharmaceutical composition comprises a pharmaceutically acceptable carrier or diluent.

In a fourth aspect, provided herein are dosage forms comprising the pharmaceutical compositions or the substantially complete vaginal microbiota preparations described herein formulated for vaginal administration, wherein the dosage form is solid, semi-solid, a liquid formulation, or a film-forming formulation.

In a fifth aspect, the invention provides a vaginal delivery system comprising a dosage form described herein, wherein said dosage form is suitable for administration to the vaginal cavity, wherein the dosage form is an applicator, dispenser or suppository.

In a sixth aspect, provided herein are methods for vaginal administration of the pharmaceutical compositions or the substantially complete vaginal microbiota preparations described herein to a human female subject, wherein the compositions or preparations are administered using a device, wherein the device comprises an open end (e.g., a tip) for insertion into the vaginal cavity, and a dispensing end (e.g., a plunger or piston) to expel the composition or preparation through the open end, the method comprising: a) introducing the open end into a vaginal cavity, b) expelling the composition or preparation into the vaginal cavity, thereby administering the composition or preparation to the vaginal cavity.

In a seventh aspect, provided herein are methods of producing a substantially complete vaginal microbiota preparation, said methods comprising A. providing a microbiota sample from a donor female genitourinary tract; wherein step A comprises one, two, or three of steps (1), (2), (3) or any combination thereof, and both steps (4) and (5): (1) adding a diluent to the microbiota sample to create a diluted sample, (2) removing a portion of the diluted microbiota sample for testing (e.g., nucleic acid sequencing), (3) pre-cooling for either refrigeration or freezing of the remainder of the microbiota sample, (4) storing the refrigerated or frozen microbiota sample under quarantine, (5) holding the refrigerated or frozen microbiota sample under quarantine until any completion of any combination of (a) testing the donor to exclude the substantial presence of one or more transmissible pathogens (e.g., blood, and/or cervicovaginal secretions, and/or urine sample testing), (b) confirming the composition and viability of the microbiota, or (c) further confirming the health of the female donor by a plurality of post-screening tests occurring within a time period of 30-90 days post-donation; and B. releasing the refrigerated or frozen microbiota sample from quarantine to define the substantially complete vaginal microbiota preparation.

In an eight aspect, provided herein are substantially complete vaginal microbiota preparations obtainable by the methods described herein.

In a ninth aspect, provided herein is an isolated substantially complete vaginal microbiota preparation suitable for vaginal administration, wherein said preparation comprises Lactobacillus iners and Lactobacillus gasseri, which together comprise about 80-99.9% of all detectable bacterial species of the preparation, and wherein the preparation comprises less than 5% of Gardnerella spp., Atopobium spp., and Prevotella spp.

In a tenth aspect, provided herein is an isolated substantially complete vaginal microbiota preparation suitable for vaginal administration, wherein said preparation comprises Lactobacillus iners and Lactobacillus jensenii which together comprise about 80-99.9% of all detectable bacterial species of the preparation, and wherein the preparation comprises less than 5% of Gardnerella spp., Atopobium spp., and Prevotella spp.

In a eleventh aspect, provided herein is an isolated substantially complete vaginal microbiota preparation suitable for vaginal administration, wherein said preparation comprises Lactobacillus iners and Lactobacillus crispatus, which together comprise about 80-99.9% of all detectable bacterial species of the preparation, and wherein the preparation comprises less than 5% of Gardnerella spp., Atopobium spp., and Prevotella spp.

In a twelfth aspect, provided herein is an isolated substantially complete vaginal microbiota preparation suitable for vaginal administration, wherein said preparation comprises Lactobacillus crispatus, Lactobacillus iners and Lactobacillus jensenii, which together comprise about 80-99.9% of all detectable bacterial species of the preparation, and wherein the preparation comprises less than 5% of Gardnerella spp., Atopobium spp., and Prevotella spp.

In a thirteenth aspect, provided herein is an isolated substantially complete vaginal microbiota preparation suitable for vaginal administration, wherein said preparation comprises Lactobacillus iners, Lactobacillus gasseri and Lactobacillus jensenii, which together comprise about 80-99.9% of all detectable bacterial species of the preparation, and further wherein the preparation comprises less than 5% of Gardnerella spp., Atopobium spp., and Prevotella spp.

In a further aspect, provided herein is a vaginal delivery system comprising; a) a dosage form suitable for administering a composition to the vaginal cavity, and b) a composition comprising a substantially complete vaginal microbiota preparation, wherein (i) the preparation comprises one to five bacterial species from the genus Lactobacillus, and (ii) the one to five bacterial species comprise about 80-99.9% of the preparation, and wherein the composition further comprises a pharmaceutically acceptable carrier or diluent. In some embodiments, the composition comprises one to four bacterial species from the genus Lactobacillus. In embodiments, the dosage form may be an applicator, dispenser or suppository. The preparation may comprise about 80-99.9% Lactobacillus crispatus.

The preparation may comprise about 80-99.9% Lactobacillus iners. The preparation may comprise about 80-99.9% Lactobacillus jensenii. The preparation may comprise about 80-99.9% Lactobacillus gasseri. The preparation may comprise about 80-99.9% of one, two, three, or four of Lactobacillus crispatus, Lactobacillus iners, Lactobacillus jensenii, and Lactobacillus gasseri.

The preparation may be free or substantially free of pathogens and pathobionts. The pathogen or pathobiont is one or more of Gardnerella spp., Atopobium spp., Prevotella spp. The preparation may comprise less than 5% of Gardnerella spp., Atopobium spp., and Prevotella spp.

The preparation may be free or substantially free of antimicrobial resistance (AMR) genes.

The preparation may be free or substantially free of human sperm (spermatozoa).

In addition to the vaginal microbiota preparation, the composition may further comprise a spermicide. The composition may further comprise an acidifying agent. The composition may further comprise lactic acid. The preparation may comprise a glycan. The glycan can be mucus.

The preparation may comprise less than 30 or less than 20 species other than from the genus Lactobacillus.

The composition may have an acidifying effect on the vaginal microbiota. The composition may have a pH of equal or less than 4.5.

The dosage form may comprise 10² to 10¹¹ CFU/VCC of lactobacilli. Preferably, the dosage form comprises at least 10⁶ CFU/VCC, at least 10⁷ CFU/VCC or at least 10⁸ CFU/VCC. The composition may have been dispensed or formulated in the dosage form. The dosage form can be sterile packaged.

The applicator or dispenser may comprise an open end (e.g., tip) for insertion into the vaginal cavity, and a dispensing end (e.g., a plunger or piston) to expel the composition.

The preparation can be obtained by a culture-independent method.

The dosage form can be calibrated (e.g., has a volume indication or maximal volume indication or control). The dosage form can be a single use or multiple use device. The dosage form can be designed from inert polymeric materials, such as polystyrene or polypropylene.

In a further aspect, provided herein is a pharmaceutical composition formulated for vaginal administration comprising a substantially complete vaginal microbiota preparation, wherein (i) the preparation comprises one to five bacterial species from the genus Lactobacillus, and (ii) the one to five bacterial species comprise about 80-99.9% of the preparation, and wherein the composition further comprises a pharmaceutically acceptable carrier or diluent. In one embodiment, the pharmaceutical composition comprises one to four bacterial species from the genus Lactobacillus. The preparation may comprise 80-99.9% Lactobacillus crispatus. The preparation may comprise 80-99.9% Lactobacillus iners. The preparation may comprise about 80-99.9% Lactobacillus jensenii. The preparation may comprise about 80-99.9% Lactobacillus gasseri. The preparation may comprise about 80-99.9% of one, two, three, or four of Lactobacillus crispatus, Lactobacillus iners, Lactobacillus jensenii, and Lactobacillus gasseri.

The preparation may further comprise further Lactobacillus species, wherein the further Lactobacillus species may be present in low relative quantities, such as 0.001%, 0,001-0,002%, 0,002-0,005%, 0.005-0.01%, 0.01-0.05%, 0.05-0.1%, or 0.1-1%.

The preparation may be free or substantially free of pathogens and pathobionts. The pathogen or pathobiont may be one or more of Gardnerella spp., Atopobium spp., and Prevotella spp. The preparation may comprise less than 5% of Gardnerella spp., Atopobium spp., and Prevotella spp. The preparation may be substantially free of antimicrobial resistance (AMR) genes. The preparation may be substantially free of human sperm (spermatozoa).

The composition may further comprise a spermicide. The composition may further comprise an acidifying agent. The composition may further comprise lactic acid. The preparation may comprise a glycan. The glycan can be mucus.

The preparation may comprise less than 20 species other than from the genus Lactobacillus.

The composition may have a pH of equal or less than 4.5.

The preparation may be obtained by a culture-independent method.

In a further aspect, provided herein is a method for vaginal administration to a human female subject in need thereof, of a composition comprising a substantially complete vaginal microbiota preparation using a device, wherein the device comprises an open end (e.g., a tip) for insertion into the vaginal cavity, and a dispensing end (e.g., a plunger or piston) to expel the composition through the open end, the method comprising: a) introducing the open end into a vaginal cavity, b) expelling the composition into the vaginal cavity, thereby administering the composition to the vaginal cavity. The device may an applicator or dispenser as described herein. The composition may be a pharmaceutical composition as described herein.

Expelling the composition may comprise contacting a mucosal or endometrial surface of the vagina. The subject is in need of improving her vaginal health. The administration to the subject may be carried out while the subject is in a lithotomy position. The composition may be of a suitable viscosity that allows the majority of it to stay in the vaginal cavity for at least 5, 10, 20, or 30 minutes, when the subject is in upright position.

The device may be calibrated (e.g., has a volume indication or maximal volume indication or control). The device can administer about 10² to 10¹¹ CFU of lactobacilli per administration. Preferably, the device can administer at least 10⁶ CFU/VCC, at least 10⁷ CFU/VCC or at least 10⁸ CFU/VCC.

The administering may be performed one, two, or three times within 3-21 days or 1 dose per cycle for one, two, three or more cycles. The administering may be performed once every one to four weeks for a period of one to six months. The device can be a single use or

The method may further comprise colonizing the vaginal mucosa with one or more Lactobacillus species. The one or more Lactobacillus species may be one, two, three, or four of Lactobacillus crispatus, Lactobacillus iners, Lactobacillus jensenii, and Lactobacillus gasseri. The preparation may comprise about 80-99.9% Lactobacillus crispatus. The preparation comprises about 80-99.9% Lactobacillus iners. The preparation may comprise about 80-99.9% Lactobacillus jensenii. The preparation may comprise about 80-99.9% Lactobacillus gasseri. The preparation may comprise about 80-99.9% of one, two, three, or four of Lactobacillus crispatus, Lactobacillus iners, Lactobacillus jensenii, and Lactobacillus gasseri.

The preparation can be substantially free of pathogens and pathobionts. The pathogen or pathobiont may be one or more of Gardnerella spp., Atopobium spp., and Prevotella spp. The preparation may comprise less than 5% of Gardnerella spp., Atopobium spp., and Prevotella spp. The preparation may be substantially free of antimicrobial resistance (AMR) genes. The preparation can be substantially free of human sperm (spermatozoa).

The composition may further comprise a spermicide. The composition may further comprise an acidifying agent. The composition may further comprise lactic acid. The preparation may comprise a glycan. The glycan can be mucus.

The preparation may comprise less than 20 species other than from the genus Lactobacillus.

The composition may have a pH of equal to or less than 4.5. The composition may have a pH effective to achieve a pH of the vaginal tract of about pH 3.5 to 4.5.

The preparation may be obtained by a culture-independent method.

In another aspect, provided herein is a kit comprising the dosage form (a) and composition (b) described above. The dosage form (a) and composition (b) can be in separate sterile packages. The dosage form can be a multi-dosage form. The dosage form (a) and/or the composition (b) may comprise multiple doses. The dosage form may be a single use or multiple use device. The composition can be freeze-dried or lyophilized. The kit may further comprise a resuspension medium for the freeze-dried or lyophilized composition.

In another aspect, provided herein is a substantially complete vaginal microbiota preparation comprising one to five, or one to four, bacterial species from the genus Lactobacillus derived from a female donor's genitourinary tract (e.g., vaginal microbial niche) secretion, wherein the one to five bacterial species comprise about 80-99.9% of the preparation and one or more species of said bacterial species are capable of engrafting (e.g., colonizing) in a female recipient's genitourinary tract. In one embodiment, the substantially complete vaginal microbiota preparation comprising one to four bacterial species from the genus Lactobacillus.

The preparation may comprise 80-99.9% of Lactobacillus species that are typically found in a healthy (e.g., non-dysbiotic) microbiome of the genitourinary tract (e.g., vaginal microbial niche). These Lactobacillus species can include Lactobacillus crispatus, Lactobacillus iners, Lactobacillus jensenii and Lactobacillus gasseri. The preparation may comprise about 80-99.9% Lactobacillus crispatus. The preparation may comprise about 80-99.9% Lactobacillus iners. The preparation may comprise about 80-99.9% Lactobacillus jensenii. The preparation may comprise about 80-99.9% Lactobacillus gasseri. The preparation may comprise about 80-99.9% of one, two, three, or four of Lactobacillus crispatus, Lactobacillus iners, Lactobacillus jensenii, and Lactobacillus gasseri.

The preparation may be formulated as a pharmaceutical composition as described herein. The pharmaceutical composition may further comprise an active agent selected from the group comprising of an antibiotic, immunological agent, a hormonal agent, metabolite or peptide. In some embodiments, the pharmaceutical composition may comprise at least one Lactobacillus strain that is added to the preparation comprised in the pharmaceutical composition. The pharmaceutical composition may be in the form of a suspension, spray, gel, cream, powder, capsule, solution for lavages, ovules, a vaginal insert, tampon, tablets or a microencapsulated product.

In another aspect, provided herein is a method of producing substantially complete vaginal microbiota preparation of any one of the preceding claims, comprising: A. providing a microbiota sample from a donor female genitourinary tract; wherein step A comprises optionally (1), (2), (3), (4), (5) or any combination thereof: (1) adding a diluent to the microbiota sample to create a diluted sample, (2) removing a portion of the diluted microbiota sample for testing (e.g., nucleic acid sequencing), (3) pre-cooling for either refrigeration or freezing of the remainder of the microbiota sample, (4) storing the refrigerated or frozen microbiota sample under quarantine, (5) holding the refrigerated or frozen microbiota sample under quarantine until any completion of any combination of (a) testing the donor to exclude the substantial presence of one or more transmissible pathogens (e.g., blood, and/or cervicovaginal secretions, and/or urine sample testing), (b) confirming the composition and viability of the microbiota, or (c) further confirming the health of the female donor by a plurality of post-screening tests occurring within a time period of 30-90 days post-donation; and B. releasing the refrigerated or frozen microbiota sample from quarantine to define substantially complete vaginal microbiota preparation.

The microbiota sample from the donor female can be a cervicovaginal secretion. The microbiota sample may comprise a glycan. The glycan can be mucus. The microbiota sample may weigh at least 100 mg, 150 mg, 200 mg or more. The microbiota sample may comprise 80-99.9% of Lactobacillus species that are typically found in a healthy (e.g., non-dysbiotic) microbiome of the genitourinary tract (e.g., vaginal microbial niche). These Lactobacillus species can include Lactobacillus crispatus, Lactobacillus iners, Lactobacillus jensenii and Lactobacillus gasseri. The microbiota sample may comprise about 80-99.9% Lactobacillus crispatus. The microbiota sample may comprise about 80-99.9% Lactobacillus iners. The microbiota sample may comprise about 80-99.9% Lactobacillus jensenii. The microbiota sample may comprise about 80-99.9% Lactobacillus gasseri. The microbiota sample may comprise about 80-99.9% of one, two, three, or four of Lactobacillus crispatus, Lactobacillus iners, Lactobacillus jensenii, and Lactobacillus gasseri.

Step B of the method may comprise determining that the microbiota sample is substantially free of pathogens and pathobionts. Step B may comprise determining that the microbiota sample is substantially free of Gardnerella spp., Atopobium spp., and Prevotella spp. Step B may comprise determining that the microbiota sample is substantially free of antimicrobial resistance (AMR) genes. Step B may comprise determining that the microbiota sample is substantially free of human sperm (spermatozoa). Step B may comprise determining that the female donor is substantially free of any one or more (two or more, three or more, or four or more) of: (i) bacteria involved in bacterial vaginosis (e.g., Gardnerella spp. and Prevotella spp.), (ii) yeast (e.g., Candida, Cryptococcus, and Saccharomyces species), (iii) sexually transmitted pathogens (e.g., Neisseria gonorrhea, Chlamydia trachomatis, and Trichomonas vaginalis), (iv) bacteria involved in urinary tract infections (e.g., E. coli, Staphylococcus, Chlamydia, and Mycoplasma), and (v) viruses (e.g., HIV, human papilloma virus (HPV), hepatitis B virus, hepatitis C virus, HSV-2), or any combination thereof.

In another aspect, provided herein is a method for restoring a healthy human vaginal microbiota balance in the female genitourinary tract of a human subject comprising administering to a subject in need of such restoration an effective amount of a pharmaceutical composition comprising a substantially complete vaginal microbiota preparation, optionally using a vaginal delivery system, thereby restoring a healthy vaginal microbiota balance. The female subject may exhibit a dysbiotic microbiota in the genitourinary tract and restoring a healthy vaginal microbiota balance provides treatment of the dysbiosis. The female subject can be treated with an effective amount of an antimicrobial agent (e.g., an antibiotic) to substantially diminish the residing dysbiotic microbiota prior to administration of the pharmaceutical composition comprising a substantially complete vaginal microbiota preparation.

In some embodiments, the female subject is subjected to a vaginal wash prior to administration of the substantially complete vaginal microbiota preparation in order to reduce the pathogen load. The vaginal wash can be performed with any suitable solution, including but not limited to saline, an acidic solution, or an antiseptic solution. In some embodiments, the acidic solution is lactic acid, optionally, wherein the lactic acid solution has a pH of about 3.5 to 4.5, optionally about pH 3.8-4.3. The antiseptic solution may be chlorhexidine or providone-iodine, wherein the povidone-iodine solution comprises about 10% or 5-10% povidone-iodine. In a particular embodiment, the vaginal wash is performed with chlorhexidine, wherein the solution comprises about 0.5% chlorhexidine.

Restoring a healthy vaginal microbiota balance may comprise colonization and engraftment of one or more species provided by the pharmaceutical composition comprising a substantially complete vaginal microbiota preparation. Restoring a healthy vaginal microbiota balance may comprise one or more of: (a) a reduction in the relative abundance of pathogen and/or pathobiont residing in the genitourinary tract (e.g., less than 10% relative abundance of pathogen and/or pathobiont), (b) an increase in relative abundance of Lactobacillus above 50% (above 60%, or above 70%), (c) a reduction in one or more pro-inflammatory markers (e.g., local or systemic), and/or (d) a lowering of vaginal pH (e.g., by at least pH 0.3, 0.5, 1.0, or 1.5), and/or (e) a change in grade based on the Ison-Hay scoring system (e.g., grade 0 or grade I), when compared to the values of (a), (b), (c), (d) or (e) determined in the same subject prior to administration of the pharmaceutical composition comprising a substantially complete vaginal microbiota preparation. Restoring a healthy vaginal microbiota balance comprises engraftment of a microbial community dominated (e.g., at least 50%, 60%, 70% or at least 80%) by one or more species of lactic acid producing bacteria.

The one or more species of lactic acid producing bacteria may be selected from: one, two, three, or four of Lactobacillus crispatus, Lactobacillus iners, Lactobacillus jensenii, and Lactobacillus gasseri.

Restoring a healthy vaginal microbiota balance may comprise achieving a pH of the vaginal tract of about pH 3.5 to 4.5. Preferably, the administration of the one or more species of lactic acid producing bacteria restores the vaginal pH to about 3.7 to about 4.2, more preferably to about 3.8 to about 4.0. The microbial community may remain dominated by lactic acid producing bacteria (e.g., lactobacilli) for at least 1 week, or at least 2, 3, 4 weeks. The microbial community may remain dominated by lactic acid producing bacteria (e.g., lactobacilli) for at least 2, 3, 4, 5 or at least 6 months.

In some embodiments, the administration of the preparation further comprises the administration of further Lactobacillus species, wherein the further Lactobacillus species may be present in low relative quantities, such as 0.001%, 0.001-0,002%, 0,002-0,005%, 0.005-0.01%, 0.01-0.05%, 0.05-0.1%, or 0.1-1%.

The method may comprise administering one or more additional active agents, selected from the group consisting of an antibiotic, immunological agent, and a hormonal agent.

The subject may be Caucasian. The subject may be Asian. The subject may be Black/African. The subject may be Hispanic. The subject may of child-bearing age or pre-menopausal. The subject may be post-menopausal.

Restoring a healthy vaginal microbiota balance may provide an antipathogenic microbial niche/vaginal environment. Restoring a healthy vaginal microbiota balance may provide an anti-inflammatory or low-inflammatory microbial niche/vaginal environment. The substantially complete vaginal microbiota preparation results in a Lactobacillus-dominated microbiota in the vaginal microenvironment, which results in a reduction of the vaginal pH, reducing the amount of pathogenic bacteria and restoration of the vaginal microbiota balance. Further, inflammation of the dysbiotic vaginal niche, even though the subjects do not present any vaginal symptoms, is treated.

In another aspect, provided herein is a method of treating dysbiosis in the female genitourinary tract of a human subject comprising administering to a subject in need of such treatment an effective amount of a pharmaceutical composition comprising a substantially complete vaginal microbiota preparation, optionally using a vaginal delivery system of any one of claims, thereby treating the dysbiosis. The dysbiosis can be associated with an infection of the genitourinary tract. The infection can be one or more of (recurrent) bacterial vaginosis (BV), vaginal candidiasis and trichomoniasis.

In another aspect, provided herein is a method of treating (chronic) inflammation in the female genitourinary tract of a human subject comprising administering to a subject in need of such treatment an effective amount of a pharmaceutical composition comprising a substantially complete vaginal microbiota preparation, optionally using a vaginal delivery system of any one of claims, thereby treating the inflammation.

In another aspect, provided herein is a method treating (recurrent) bacterial vaginosis (BV) in the female genitourinary tract of a human subject comprising administering to a subject in need of such treatment an effective amount of a pharmaceutical composition comprising a substantially complete vaginal microbiota preparation, optionally using a vaginal delivery system, thereby treating (recurrent) BV.

In another aspect, provided herein is a method of treating vaginal candidiasis in the female genitourinary tract of a human subject comprising administering to a subject in need of such treatment an effective amount of a pharmaceutical composition comprising a substantially complete vaginal microbiota preparation, optionally using a vaginal delivery system, thereby treating vaginal candidiasis.

In another aspect, provided herein is a method of treating trichomoniasis in the female genitourinary tract of a human subject comprising administering to a subject in need of such treatment an effective amount of a pharmaceutical composition comprising a substantially complete vaginal microbiota preparation, optionally using a vaginal delivery system, thereby treating trichomoniasis.

The methods described herein may further comprise administering one or more additional active agents, selected from the group consisting of an antibiotic, anti-mycotic, immunological agent, and a hormonal agent.

### BRIEF DESCRIPTION OF THE FIGURES

FIG. 1 is a schematic representation of the donor program to produce substantially complete vaginal microbiota preparations. Donors were selected based on microbiome sequencing, a medical examination, and the absence of certain diseases. Testing was performed both before and after the donation visits. All samples provided by the donor were subjected to quality control (see, e.g., FIG. 5). Samples were released only when donor and samples passed all requirements.

FIG. 2 shows the distribution of vaginal microbiomes from a cohort of 96 female subjects that were assessed by shotgun DNA sequencing to identify suitable donors from which to obtain cervicovaginal secretions. Relative abundance of bacteria was measured, and subjects were classified as “healthy”, “dysbiotic” and “undefined” using the classification parameters indicated.

FIG. 3 shows stacked bar graphs of relative bacterial abundance in the donors from the cohort of 96 female subjects (see, FIG. 2) with a healthy vaginal microbiome (n=61), as assessed by shotgun sequencing. All donor microbiomes in this graph contain at least 80% of vaginal Lactobacillus species (Lactobacillus crispatus, Lactobacillus gasseri, Lactobacillus jensenii, and Lactobacillus iners) or mixtures thereof, and less than 5% selected pathogens (Atopobium spp., Prevotella spp., B. vaginale, and F. vaginae) or mixtures thereof.

FIG. 4 shows stacked bar graphs of relative bacterial abundance in the donors from the cohort of 96 female subjects (see, FIG. 2) with a dysbiotic vaginal microbiome (left, n=27) or undefined microbiome (right, n=8) as assessed by shotgun sequencing. Dysbiotic microbiomes contain at least 20% species from selected vaginal pathogens (Atopobium spp., Prevotella spp., B. vaginale, and F. vaginae) or mixtures thereof, and less than 10% of vaginal Lactobacillus species (Lactobacillus crispatus, Lactobacillus gasseri, Lactobacillus jensenii, and Lactobacillus iners) or mixtures thereof.

FIG. 5 shows a flowchart depicting an exemplary procedure to obtain cervicovaginal secretions and processing thereof. FIG. 5A provides a general overview, whereas FIG. 5B provides a concrete exemplary embodiment of the procedure. The secretions were split into samples for characterization and quality control (including, e.g., viability count, DNA sequencing and pathogen screening) and processing into a substantially complete vaginal microbiota preparation.

FIG. 6A-6C show stacked bar graphs of four main Lactobacillus species present in the substantially complete vaginal microbiota preparations from 9 donors (A-I) as assessed by shotgun sequencing. The results from eight representative donation visits are shown for each donor. Where present, the vertical line indicates a new round of visits as recurring donor.

FIG. 7 shows plots depicting stability of substantially complete vaginal microbiota preparations after storage in −80° C. for up to 11 months. Each data point represents one sample; each connecting line indicates samples from one donation visit of one donor. Similar symbols and shading indicate similar microbiomes.

FIG. 8 shows bar graphs of substantially complete vaginal microbiota preparations' viability after thawing from −80° C. A. Gradual thawing of the samples and warming them up until approx. 30° C. B. Effect of repeated freeze-thaw cycles or prolonged incubation at ambient temperature (RT=room temperature) on cell viability.

FIG. 9A-9B show stacked bar graphs of relative bacterial abundance in two recipients (A-B) of a substantially complete vaginal microbiota preparation and their respective donors, as measured by shotgun sequencing. All recipients were enrolled based on their microbiome at screening; a baseline microbiome sample was taken just prior to dosing. The asterisk (*) indicates that one menstruation took place before that sample was taken. Two asterisks (**) indicate that two menstrual cycles passed.

FIG. 10 shows a principal component analysis (PCA) plot for two recipients of a substantially complete vaginal microbiota preparation, pre- and post-dosing, and their respective donors. The variables used as input are the relative abundances of bacterial species as measured by shotgun sequencing. The same input was used as for the graphs in FIG. 9.

FIG. 11 shows plots of a panel of inflammatory markers in a screening cohort (n=60), comparing women with a dysbiotic (n=19) and healthy (n=41) microbiome. A. PCA plot, where each dot corresponds to one woman; the 92 inflammation panel markers were used as variables. The asterisk (*) indicates a woman with a healthy microbiome who might have an unrecognized infection such as, e.g., Candida. B. Box and whisker plot of selected inflammatory markers which are associated with bacterial vaginosis.

FIG. 12 shows plots of inflammatory markers from the inflammation panel described in FIG. 11 in recipient 2 pre- and post-dosing with a substantially complete vaginal microbiota preparation. A. PCA plot showing the shift in inflammation state in the recipient pre- and post-dosing, relative to the donor samples and with a subset of the screening cohort for comparison. The 92 inflammation panel markers were used as variables. B. Dot plots for recipient 2 pre- and post-dosing, showing the same selected inflammatory markers as in FIG. 11B for the screening cohort. Horizontal lines represent medians; each dot represent a technical replicate: for each time point, 2 aliquots of the same sample were used, which were ran in duplicate.

FIG. 13 shows exemplary vaginal delivery systems (100) in three different set-ups (FIGS. 13A, 13B and 13C). FIG. 13A shows a vaginal delivery system in a pre-filled configuration. The basic dispensing device (102) comprises: a dispensing end (e.g., a plunger or piston) (101), which optionally can be configured manually or electromechanically; and an open end (e.g., a tip) (105) for insertion into the vaginal cavity, which optionally can be molded in any suitable form to better insert into the vaginal cavity. Optionally, the device (102) comprises a type of calibration (104), e.g., has a volume indication or maximal volume indication or control, e.g., to aide dosing. In a pre-filled configuration, the composition comprising the substantially complete vaginal microbiota preparation (103) (e.g., in frozen or liquid form) is comprised in the dispensing device (102). To administer the composition (103), the dispensing end (101) is pushed by some force into the dispensing device (102) and the composition is expelled through the open end (105), e.g., into the vaginal cavity. If the content is provided in frozen form, the user would first thaw the frozen content (103) prior to administration.

FIG. 13B shows a vaginal delivery system (100) in an alternative configuration. The dispensing device (102) is not pre-filled but empty. The composition comprising the substantially complete vaginal microbiota preparation (103) is provided separately from the dispensing device (102), e.g., in a container (107) with a cap (106), such as a cryo-vial or other suitable storage container. The dispensing device and the container can be packaged, shipped and or stored separately. A user would then, usually shortly prior to use (e.g., administration to the urogenital tract) dispense the content (103) of the container (107) and dispense the content into the dispensing device (102). If desired, the content (103) can be frozen, in which case the user would thaw the frozen content (103) prior to administration, or the content (103) can be provided in lyophilized form, in which case the user would rehydrate the lyophilized content (103) prior to administration. The dispensing end (101) may be provided as detached from the dispensing device (102), as shown, or can, alternatively be attached to it and is later removed just prior to filling the dispensing device (102) with the composition comprising the substantially complete vaginal microbiota preparation (103).

FIG. 13C shows a vaginal delivery system (100) in an alternative configuration. The dispensing device (108) comprises a dispensing end (101), and an open end (105), however, unlike (102), the dispensing device (108) comprises a docking platform (110) that allows for a connection with a container (109), such as a cartridge, e.g., to allow a quick-connection. Optionally, the container (109) can be loaded into the dispensing device (108), e.g., in a suitable space between the docking platform (110) and the dispensing end (101). The container (109) comprises the composition comprising the substantially complete vaginal microbiota preparation (103), optionally a type of calibration (104), and caps (106) on both ends. One end of the container (109) may then be connected to the docking platform (110) of the dispensing device (108) and the other end to the dispensing end (101). The caps (106) of the container (109) may be configured so that their seal is broken upon contacting with the docking platform (110) and the dispensing end (101), e.g., they would be made from a soft material (such as, e.g., a rubber or other soft polymer or a film) that can be pierced by both the docking platform (110) and the dispensing end (101) to release the content (103).

All configurations can be adapted for single use or multiple use applications. For example, the vaginal delivery system (100) exemplified in FIG. 13C could be adapted to multiple use by keeping the dispensing end (101) of the vaginal delivery system permanently attached to the dispensing device (108, dashed lines) and configure (105) and/or (110) so that they are discarded after each use or made of a cleanable/sterilizable multi-use material and configured to be removable from the device (108).

FIG. 14 depicts the design of a first clinical study. This study explores and maps the shifts in vaginal microbiomes when 3 sequential administrations of the substantially complete vaginal microbiota preparations to recipients are preformed. Recipients are screened to have vaginal dysbiosis based on criteria defined from metagenomic sequencing of a vaginal sample.

FIG. 15 is a schematic outlining an exemplary design of a clinical study that administers an antibiotic with the substantially complete vaginal microbiota preparation.

FIG. 16 depicts the design of a second clinical study. This study explores and maps the shifts in vaginal microbiomes when up to 3 administrations of substantially complete vaginal microbiota preparations to recipients are preformed, separated by follow-up visits and assessment of treatment success. Recipients are screened to have vaginal dysbiosis based on criteria defined from metagenomic sequencing of a vaginal sample.

### DETAILED DESCRIPTION

The present invention relates to compositions, devices, and kits comprising substantially complete vaginal microbiota preparation and methods of making and using the same. Provided herein are substantially complete vaginal microbiota preparations and compositions for modulation of the vaginal microbiota for prevention or treatment of conditions and diseases where the composition of the vaginal microbiota has been implicated. The applicant has developed a safe and efficacious process to produce substantially complete vaginal microbiota preparations and provides devices and methods for administering and using same, e.g., to restore or maintain a healthy human vaginal microbiota balance in the female genitourinary tract, to treat dysbiosis and/or inflammatory conditions.

Vaginal Flora, Vaginal Microbial Niche

A healthy vaginal flora is characterized by an acidic environment inhabited predominantly by lactic acid bacteria, primarily species of Lactobacillus (residing in the vaginal microbial niche). The microbial composition in healthy women can differ, though it is typically dominated by one of four Lactobacillus species: L. crispatus, L. iners, L. gasseri, L. jensenii, and mixtures thereof. A healthy vagina of a women of child-bearing age is estimated to be dominated by 10⁷-10⁹ colony forming units of lactic acid producing bacteria (e.g., Lactobacillus) per gram of fluid. The species distribution differs between women of different geographical background, race (e.g., Asian, white women, black, Hispanic), age, lifestyle and the like. The composition of the vaginal flora is also influenced by which specific strains and/or species the woman has inherited from her mother and/or which strains and/or species have migrated from her digestive tract to the urogenital tract. Healthy, fertile women present with a pH of about 3 to 5.5 (more specifically between pH 3.5 and 4.5) in the vagina, primarily as a result of lactic acid production. Vaginal pH undergoes physiological changes from birth to menopause. The increase of vaginal pH above 4.0-4.5 is detrimental for the survival of Lactobacillus bacteria, but not for other microorganisms. The vaginal lactobacilli are believed to have a protective effect against vaginal colonization by pathogenic microorganisms (e.g., yeast (Candida albicans), Trichomonas vaginalis, Neisseria gonorrhoeae, and Chlamydia trachomatis, and viruses, e.g., HIV, HSV-2, and various anaerobes) and prevent the vaginal establishment of, for instance, bacteria that are present in the colon, such as Gardnerella vaginalis, Mobiluncus, Bacteroides, Prevotella and Escherichia coli.

Several factors may contribute to the disturbance of the vaginal flora. Factors may include, a) use of antibiotics to kill pathogenic bacteria which can lead to significantly reduced levels of lactobacilli in the vagina; b) hormonal changes, in particular changes in estrogen levels, which are observed in several phases of a woman's life (e.g., puberty, pregnancy, childbearing age, pre- and post-menopause); estrogen levels are thought to be associated with Lactobacillus levels (dominance) in the vagina; c) sexual intercourse, which can be associated with pH increases (semen generally is alkaline) that may disturb the vaginal flora, because bacteria other than lactobacilli may start to flourish once the vaginal pH increases; d) use of medications, e.g., chemotherapeutics or antimycotics; e) use of birth control products; f) during menstruation; g) insufficient hygiene (e.g., promoting undesirable spread of the microorganisms from rectum to the urogenital area); h) general health status, such as, e.g., being diabetic.

Disturbance of the vaginal flora may lead to vaginal dysbiosis and vaginal disorders, e.g., candidiasis and bacterial vaginosis, which are two common vaginal disorders that affect women worldwide. Bacterial vaginosis is believed to be the result of displaced vaginal lactic acid producing bacteria which are replaced by a range of unwanted species such as Gardnerella vaginalis, Bacteroides, Mobiluncus, Prevotella, and Mycoplasma hominis. Vaginal infections are most often associated with one or more of: Escherichia, Enterococcus, Pseudomonas, Proteus, Klebsiella, Streptococcus, Staphylococcus, Gardnerella, Ureaplasma, Bacteroides, Peptococcus, Neisseria, Serratia, Corynebacterium, Clostridium, and Candida.

Vaginal lactobacilli predominance is thought to play an important role in resistance to infection via production of lactic acid and acidification of the vagina and by production of other antimicrobial products, such as, e.g., hydrogen peroxide. The presence of lactobacilli in the vagina has been linked to decreased frequencies of bacterial vaginosis, yeast vaginitis and sexually transmitted pathogens, including Neisseria gonorrhea, Chlamydia trachomatis, and Trichomonas vaginalis. Lactobacillus dominance varies among ethnic groups (they are thought to be very predominant in Asian and white women but less so in black and Hispanic women, though they still represent the majority).

Studies have shown that Lactobacillus-depleted communities can be transient, lasting just a few days, while in other instances the depleted communities persist for many weeks. Some women with Lactobacillus-depleted communities remain asymptomatic and healthy. However, such women may be at higher risk for infections and sexually transmitted diseases (STDs).

In some embodiments, the vaginal flora is restored by replenishing or supplementing the disturbed vaginal flora with the substantially complete vaginal microbiota preparation and composition thereof described herein.

Substantially Complete Vaginal Microbiota Preparation and Lactobacilli

Aspects of the invention relate to substantially complete vaginal microbiota preparations, methods of making and using the same, and pharmaceutical compositions comprising the same. There has been considerable interest in the development of non-antibiotic, ecologically appropriate approaches to replenish healthy vaginal flora in female subjects in need thereof. The substantially complete vaginal microbiota preparations described herein provide a solution for problems encountered in the past with using isolated and culture-propagated defined or single strain compositions or those containing multiple isolated and culture-propagated strains. Problems include lack of vaginal colonization and engraftment in the urogenital tract, including the vaginal cavity, of the strains that have been administered. The substantially complete vaginal microbiota preparations described herein can be, e.g., administered to the vaginal cavity to modulate the vaginal microbial niche for maintenance of a healthy vaginal microbiota and to help restore an unbalanced vaginal microbiota. It was found that a substantially complete vaginal microbiota preparation is preferable and advantageous to composition containing single and culture-propagated strains in that it is capable of efficiently colonizing the vaginal microbial niche upon administration.

The female urogenital (also known as genital-urinary) tract consists of interconnected biogeographical niches. Bacteria from the vaginal microbial community can migrate through the cervix to remote sites of the urogenital tract. A dysbiosis in the vaginal microbial community can result in a dysbiosis in remote sites. Dysbiosis at these sites has been associated with a range of disease and conditions, including urinary tract infection (UTI) and pelvic inflammatory disease (PID). In some embodiments, administration of a substantially complete vaginal microbiota preparation to the vagina includes resolving dysbiosis in remote sites of the urogenital tract.

In some embodiments, the substantially complete vaginal microbiota preparations comprise one, two, three, four or five different bacterial species from the genus Lactobacillus. In some embodiments, the substantially complete vaginal microbiota preparations comprise one, two, three, or four different bacterial species from the genus Lactobacillus. In some embodiments, the bacterial species comprise about 80%, about 85%, about 90%, about 91%, about 92%, about 93%, about 94%, about 95%, about 96%, about 97%, about 98%, about 99%, about 99.5%, about 99.9%, 80-99%, 75%-95%, 85%-95%, 85%-99%, or 90%-99% of the preparation (of the total of all detectable bacterial taxa (e.g., species) of the preparation).

In some embodiments, the preparation comprises of one of (a) to (o) Lactobacillus species and species combinations: a) Lactobacillus crispatus; (b) Lactobacillus iners; (c) Lactobacillus jensenii; (d) Lactobacillus gasseri; (e) Lactobacillus crispatus and Lactobacillus iners; (f) Lactobacillus crispatus and Lactobacillus jensenii; (g) Lactobacillus crispatus and Lactobacillus gasseri; (h) Lactobacillus iners and Lactobacillus jensenii; (i) Lactobacillus iners and Lactobacillus gasseri; (j) Lactobacillus jensenii and Lactobacillus gasseri; (k) Lactobacillus crispatus, Lactobacillus iners and Lactobacillus jensenii; (1) Lactobacillus crispatus, Lactobacillus iners and Lactobacillus gasseri; (m) Lactobacillus crispatus, Lactobacillus jensenii and Lactobacillus gasseri; (n) Lactobacillus iners, Lactobacillus jensenii and Lactobacillus gasseri; (o) Lactobacillus crispatus, Lactobacillus iners, Lactobacillus jensenii and Lactobacillus gasseri.

In one aspect, the substantially complete vaginal microbiota preparation (i) comprises one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the preparation; and (ii) comprises less than 5% of Gardnerella spp., Atopobium spp., and Prevotella spp.; wherein the pharmaceutical composition comprises a pharmaceutically acceptable carrier or diluent.

In some embodiments, about 80-99.9% of all detectable bacterial species of the preparation comprise one bacterial species from the genus Lactobacillus, selected from Lactobacillus crispatus, Lactobacillus iners, Lactobacillus jensenii, and Lactobacillus gasseri. In some embodiments, about 80-99.9% of all detectable bacterial species of the preparation comprise Lactobacillus crispatus. In some embodiments, about 80-99.9% of all detectable bacterial species of the preparation comprise Lactobacillus iners. In some embodiments, about 80-99.9% of all detectable bacterial species of the preparation comprise Lactobacillus jensenii. In some embodiments, about 80-99.9% of all detectable bacterial species of the preparation comprise Lactobacillus gasseri.

In some embodiments, about 80-99.9% of all detectable bacterial species of the preparation comprise two bacterial species from the genus Lactobacillus, selected from Lactobacillus crispatus, Lactobacillus iners, Lactobacillus jensenii, and Lactobacillus gasseri. In some embodiments, about 80-99.9% of all detectable bacterial species of the preparation comprise Lactobacillus crispatus and Lactobacillus iners. In some embodiments, about 80-99.9% of all detectable bacterial species of the preparation comprise Lactobacillus crispatus and Lactobacillus jensenii. In some embodiments, about 80-99.9% of all detectable bacterial species of the preparation comprise Lactobacillus crispatus and Lactobacillus gasseri. In some embodiments, about 80-99.9% of all detectable bacterial species of the preparation comprise Lactobacillus iners and Lactobacillus jensenii. In some embodiments, about 80-99.9% of all detectable bacterial species of the preparation comprise Lactobacillus iners and Lactobacillus gasseri.

In some embodiments, about 80-99.9% of all detectable bacterial species of the preparation comprise three bacterial species from the genus Lactobacillus, selected from Lactobacillus crispatus, Lactobacillus iners, Lactobacillus jensenii, and Lactobacillus gasseri.

In some embodiments, about 80-99.9% of all detectable bacterial species of the preparation comprise Lactobacillus crispatus, Lactobacillus iners and jensenii. In some embodiments, about 80-99.9% of all detectable bacterial species of the preparation comprise Lactobacillus crispatus, Lactobacillus iners and gasseri.

In some embodiments, about 80-99.9% of all detectable bacterial species of the preparation comprise four bacterial species from the genus Lactobacillus, selected from Lactobacillus crispatus, Lactobacillus iners, Lactobacillus jensenii, and Lactobacillus gasseri.

In some embodiments, Lactobacillus crispatus or Lactobacillus iners are present in greater relative quantities than other bacterial species of the preparation, for example, 80-99.9% of all detectable bacterial species of the substantially complete vaginal microbiota preparation may be Lactobacillus crispatus, further comprising less than 20%, 10%, 5%, 2%, 1%, 0.5% or 0.1% other bacterial species.

Further Lactobacillus Species

In some embodiments, one or more additional Lactobacillus species are present in the preparations in minor quantities (e.g., less than 20%, 15%, 10%, 5%, 2%, 1% of the Lactobacillus species of the preparation). In some embodiments, these lactobacilli species are present in a concentration of about 0.01-1%, 0.02-0.5% or 0.01-0.3% of all detectable bacterial species of a substantially complete vaginal microbiota preparation.

In a further embodiment, the substantially complete vaginal microbiota preparation comprises further lactobacilli other than Lactobacillus crispatus, Lactobacillus iners, Lactobacillus jensenii, or Lactobacillus gasseri, wherein the further Lactobacillus species include, e.g., Lactobacillus acidophilus, Limosilactobacillus fermentum (formerly known as Lactobacillus fermentum), Lacticaseibacillus casei (formerly known as Lactobacillus casei), and Lacticaseibacillus rhamnosus (formerly known as Lactobacillus rhamnosus). As used herein, Limosilactobacillus fermentum, Lacticaseibacillus casei, Lacticaseibacillus rhamnosus are referred to as Lactobacillus and are included in the meaning of Lactobacillus.

The substantially complete vaginal microbiota preparation of the invention comprises less than 5% of Gardnerella spp., Atopobium spp., and Prevotella spp. In some embodiments, substantially complete vaginal microbiota preparation of the invention comprises less than 10%, less than 7%, less than 5%, less than 2%, less than 1%, less than 0.5%, or less than 0.1% of Gardnerella spp., Atopobium spp., and Prevotella spp. The preparation of the invention can further comprise less than 5% of Gardnerella vaginalis., Atopobium spp., and Prevotella spp. and Fannyhessa vaginae. Gardnerella vaginalis was historically misclassified and has recently been re-classified as belonging to the genus Bifidobacteria. The terms Gardnerella vaginalis and Bifidobacterium vaginalis are thus used interchangeably. Atopobium vaginae and Fannyhessea vaginea are also used interchangeably throughout the application.

Provided herein are substantially complete vaginal microbiota preparations, which are separate from the animal or human body (isolated substantially complete vaginal microbiota preparations). The substantially complete vaginal microbiota preparation is thus used interchangeably with isolated substantially complete vaginal microbiota preparation.

Lactobacillus crispatus-Dominant Preparations

Provided herein are Lactobacillus crispatus-dominant substantially complete vaginal microbiota preparations, wherein Lactobacillus crispatus is present in a greater amount than each of Lactobacillus iners, Lactobacillus jensenii and Lactobacillus gasseri. Preferred aspects and embodiments of Lactobacillus crispatus-dominant substantially complete vaginal microbiota preparations are provided in the following.

In one aspect, the substantially complete vaginal microbiota preparation of the invention comprises Lactobacillus crispatus, in a relative quantity of about 80-99.9% of all detectable bacterial species of the preparation; and less than 5% of Gardnerella spp., Atopobium spp., and Prevotella spp. In some embodiments, preparation comprises Lactobacillus crispatus, in a relative quantity of above 90%, 95%, 99% or 99.9% of all detectable bacterial species of the preparation. In some embodiments, the species Lactobacillus iners, Lactobacillus jensenii, Lactobacillus gasseri are not present in detectable quantities in the preparation.

In another aspect, the substantially complete vaginal microbiota preparation of the invention comprises Lactobacillus crispatus and Lactobacillus iners, which together comprise about 80-99.9% of all detectable bacterial species of the preparation; and less than 5% of Gardnerella spp., Atopobium spp., and Prevotella spp. In some embodiments, the preparation does not comprise detectable quantities of Lactobacillus gasseri and/or Lactobacillus jensenii. In some embodiments, the preparation comprises Lactobacillus crispatus and Lactobacillus iners which together comprise about 80-99.9% of all detectable bacterial species of the preparation, wherein Lactobacillus iners comprises up to 5%, 10%, 20% or 30% of all detectable bacterial species of the preparation. In some embodiments, the preparation of the invention comprises Lactobacillus crispatus and Lactobacillus iners which together comprise about 80-99.9% of all detectable bacterial species of the preparation, wherein Lactobacillus iners comprises about 5-40%, such as 5-10%, 10-20%, 20-30%, or 30-40% of all detectable bacterial species of the preparation. In some embodiments, the preparation comprises Lactobacillus crispatus and Lactobacillus iners which together comprise about 80-99.9% of all detectable bacterial species of the preparation, wherein Lactobacillus iners comprises at least 5%, 10%, 20% or 30% of all detectable bacterial species of the preparation.

In one aspect, the substantially complete vaginal microbiota preparation of the invention comprises Lactobacillus crispatus and Lactobacillus jensenii, which together comprise about 80-99.9% of all detectable bacterial species of the preparation; and less than 5% of Gardnerella spp., Atopobium spp., and Prevotella spp. In some embodiments, the preparation does not comprise detectable quantities of Lactobacillus gasseri and/or Lactobacillus iners. In some embodiments, the preparation comprises Lactobacillus crispatus and Lactobacillus jensenii, which together comprise about 80-99.9% of all detectable bacterial species of the preparation, wherein Lactobacillus jensenii comprises at least 0.01 to about 45% of all detectable bacterial species of the preparation. In some embodiments, the preparation of the invention comprises Lactobacillus crispatus and Lactobacillus jensenii, which together comprise about 80-99.9% of all detectable bacterial species of the preparation, wherein Lactobacillus jensenii comprises about 0.01-5%, 5-10%, 10-20%, or 30-40% of all detectable bacterial species of the preparation. In some embodiments, the preparation comprises Lactobacillus crispatus and Lactobacillus jensenii which together comprise about 80-99.9% of all detectable bacterial species of the preparation, wherein Lactobacillus jensenii comprises about 0.01-5% of all detectable bacterial species of the preparation.

In one aspect, the substantially complete vaginal microbiota preparation of the invention comprises Lactobacillus crispatus, Lactobacillus iners and Lactobacillus jensenii which together comprise about 80-99.9% of all detectable bacterial species of the preparation; and less than 5% of Gardnerella spp., Atopobium spp., and Prevotella spp. In some embodiments, the preparation does not comprise detectable quantities of Lactobacillus gasseri. In some embodiments, the preparation comprises Lactobacillus crispatus, Lactobacillus iners and Lactobacillus jensenii, which together comprise about 80-99.9% of all detectable bacterial species of the preparation, wherein each of Lactobacillus iners and Lactobacillus jensenii comprises at least 0.01 to about 20% of all detectable bacterial species of the preparation. In some embodiments, the preparation comprises Lactobacillus crispatus, Lactobacillus iners and Lactobacillus jensenii, which together comprise about 80-99.9% of all detectable bacterial species of the preparation, wherein each of Lactobacillus iners and Lactobacillus jensenii comprises at least 0.01 to about 15% of all detectable bacterial species of the preparation. In some embodiments, the preparation comprises Lactobacillus crispatus, Lactobacillus iners and Lactobacillus jensenii, which together comprise about 80-99.9% of all detectable bacterial species of the preparation, wherein each of Lactobacillus iners and Lactobacillus jensenii comprises about 2-15% of all detectable bacterial species of the preparation. In some embodiments, the preparation comprises Lactobacillus crispatus, Lactobacillus iners and Lactobacillus jensenii, which together comprise about 80-99.9% of all detectable bacterial species of the preparation, wherein each of Lactobacillus iners and Lactobacillus jensenii comprises about 2-10% of all detectable bacterial species of the preparation. In some embodiments, the preparation of the invention comprises Lactobacillus crispatus, Lactobacillus iners and Lactobacillus jensenii, which together comprise about 80-99.9% of all detectable bacterial species of the preparation, wherein Lactobacillus jensenii comprises about 0.01-5%, 5-10%, or 10-20% of all detectable bacterial species of the preparation. In some embodiments, the preparation comprises Lactobacillus crispatus Lactobacillus iners and Lactobacillus jensenii, which together comprise about 80-99.9% of all detectable bacterial species of the preparation, wherein Lactobacillus iners comprises about 0.01-5%, 5-10%, or 10-20% of all detectable bacterial species of the preparation. In some embodiments, the preparation comprises Lactobacillus crispatus Lactobacillus iners and Lactobacillus jensenii, which together comprise about 80-99.9% of all detectable bacterial species of the preparation, wherein each of Lactobacillus iners and Lactobacillus jensenii comprises up to 15% of all detectable bacterial species.

In one aspect, the substantially complete vaginal microbiota preparation of the invention comprises Lactobacillus crispatus, Lactobacillus iners, Lactobacillus jensenii and Lactobacillus gasseri, which together comprise about 80-99.9% of all detectable bacterial species of the preparation; and less than 5% of Gardnerella spp., Atopobium spp., and Prevotella spp. In some embodiments, the preparation comprises Lactobacillus crispatus, Lactobacillus iners, Lactobacillus jensenii and Lactobacillus gasseri, which together comprise about 80-99.9% of all detectable bacterial species of the preparation, wherein Lactobacillus crispatus comprise at least 50%, 60%, 70% or 80% of all detectable bacterial species of the preparation. In some embodiments, the preparation comprises Lactobacillus crispatus, Lactobacillus iners, Lactobacillus jensenii and Lactobacillus gasseri, which together comprise about 80-99.9% of all detectable bacterial species of the preparation, wherein each of Lactobacillus jensenii, Lactobacillus iners and Lactobacillus gasseri comprises 0.01-20%, 0.2-15% or 0.2-10% of all detectable bacterial species of the preparation. In some embodiments, the preparation comprises Lactobacillus crispatus, Lactobacillus iners, Lactobacillus jensenii and Lactobacillus gasseri, which together comprise about 80-99.9% of all detectable bacterial species of the preparation, wherein each of Lactobacillus jensenii, Lactobacillus iners and Lactobacillus gasseri comprises 0.01 to about 5% of all detectable bacterial species of the preparation. In some embodiments, the preparation comprises Lactobacillus crispatus, Lactobacillus iners, Lactobacillus jensenii and Lactobacillus gasseri, which together comprise about 80-99.9% of all detectable bacterial species of the preparation, wherein Lactobacillus crispatus comprises at least 60% of all detectable bacterial species of the preparation, and further wherein each of Lactobacillus jensenii, Lactobacillus crispatus and Lactobacillus gasseri comprises 0.01 to about 10% of all detectable bacterial species of the preparation.

Lactobacillus iners-Dominant Preparations

The invention further provides Lactobacillus iners-dominant substantially complete vaginal microbiota preparations, wherein Lactobacillus iners is present in a greater amount than each of Lactobacillus crispatus, Lactobacillus jensenii and Lactobacillus gasseri. Preferred aspects and embodiments of Lactobacillus iners-dominant substantially complete vaginal microbiota preparations are provided in the following.

In one aspect, the substantially complete vaginal microbiota preparation of the invention comprises Lactobacillus iners, in a relative quantity of about 80-99.9% of all detectable bacterial species of the preparation; and less than 5% of Gardnerella spp., Atopobium spp., and Prevotella spp. In some embodiments, preparation comprises Lactobacillus iners, in a relative quantity of above 90%, 95%, 99% or 99.9% of all detectable bacterial species of the preparation. In some embodiments, the species Lactobacillus crispatus, Lactobacillus jensenii, Lactobacillus gasseri are not present in detectable quantities in the preparation.

In another aspect, the substantially complete vaginal microbiota preparation of the invention comprises Lactobacillus iners and Lactobacillus crispatus, which together comprise about 80-99.9% of all detectable bacterial species of the preparation; and less than 5% of Gardnerella spp., Atopobium spp., and Prevotella spp. In some embodiments, the preparation does not comprise detectable quantities of Lactobacillus gasseri. In some embodiments, the preparation comprises Lactobacillus iners and Lactobacillus crispatus, which together comprise about 80-99.9% of all detectable bacterial species of the preparation, wherein Lactobacillus crispatus comprises less than 20%, less than 10% or less than 5% of all detectable bacterial species of the preparation. In some embodiments, the preparation of the invention comprises Lactobacillus iners and Lactobacillus crispatus, which together comprise about 80-99.9% of all detectable bacterial species of the preparation, wherein Lactobacillus crispatus comprises 0.01-10%, 0.01-5%, 0.01-2% or 0.01-1%, of all detectable bacterial species of the preparation. In some embodiments, the preparation comprises Lactobacillus iners and Lactobacillus crispatus, which together comprise about 80-99.9% of all detectable bacterial species of the preparation, wherein Lactobacillus iners comprises at least 50%, 60%, 70%, 80%, 90%, 95%, or 99% of all detectable bacterial species of the preparation. In some embodiments, the preparation comprises Lactobacillus iners and Lactobacillus crispatus, which together comprise about 80-99.9% of all detectable bacterial species of the preparation, wherein Lactobacillus iners comprises at least 70% and Lactobacillus crispatus comprises less than 20% of all detectable bacterial species of the preparation.

In another aspect, the substantially complete vaginal microbiota preparation of the invention comprises Lactobacillus iners and Lactobacillus jensenii, which together comprise about 80-99.9% of all detectable bacterial species of the preparation; and less than 5% of Gardnerella spp., Atopobium spp., and Prevotella spp. In some embodiments, the preparation does not comprise detectable quantities of Lactobacillus gasseri and/or Lactobacillus crispatus. In some embodiments, the preparation comprises Lactobacillus iners and Lactobacillus jensenii, which together comprise about 80-99.9% of all detectable bacterial species of the preparation, wherein Lactobacillus jensenii comprises less than 1%, 0.5%, 0.2%, 0.1%, 0.05% or 0.01% of all detectable bacterial species of the preparation. In some embodiments, the preparation comprises Lactobacillus iners and Lactobacillus jensenii, which together comprise about 80-99.9% of all detectable bacterial species of the preparation, wherein Lactobacillus jensenii comprises 0.01-0.05% of all detectable bacterial species of the preparation. In some embodiments, the preparation comprises Lactobacillus iners and Lactobacillus jensenii, which together comprise about 80-99.9% of all detectable bacterial species of the preparation, wherein Lactobacillus iners comprises at least 70% and Lactobacillus jensenii comprises less than 1% of all detectable bacterial species of the preparation. In some embodiments, the preparation comprises Lactobacillus iners and Lactobacillus jensenii, which together comprise about 80-99.9% of all detectable bacterial species of the preparation, wherein Lactobacillus iners comprises at least 80% and Lactobacillus jensenii comprises less than 0.1% of all detectable bacterial species of the preparation. In some embodiments, the preparation comprises Lactobacillus iners and Lactobacillus jensenii, which together comprise about 80-99.9% of all detectable bacterial species of the preparation, wherein Lactobacillus iners comprises at least 90% and Lactobacillus jensenii comprises less than 0.1% of all detectable bacterial species of the preparation.

In another aspect, the substantially complete vaginal microbiota preparation of the invention comprises Lactobacillus iners and Lactobacillus gasseri, which together comprise about 80-99.9% of all detectable bacterial species of the preparation; and less than 5% of Gardnerella spp., Atopobium spp., and Prevotella spp. In some embodiments, the preparation does not comprise detectable quantities of Lactobacillus jensenii and/or Lactobacillus crispatus. In some embodiments, Lactobacillus jensenii and/or Lactobacillus crispatus comprise less than 2%, 1%, 0.5%, 0.1% of all detectable bacterial species of the preparation. In some embodiments, the preparation comprises Lactobacillus iners and Lactobacillus gasseri, which together comprise about 80-99.9% of all detectable bacterial species of the preparation, wherein Lactobacillus gasseri comprises less than 5%, 4%, 3%, 2%, or 1% of all detectable bacterial species of the preparation. In some embodiments, the preparation comprises Lactobacillus iners and Lactobacillus gasseri, which together comprise about 80-99.9% of all detectable bacterial species of the preparation, wherein Lactobacillus gasseri comprises 0.01-5%, 1-4%, or 1-3% of all detectable bacterial species of the preparation. In some embodiments, the preparation comprises Lactobacillus iners and Lactobacillus gasseri, which together comprise about 80-99.9% of all detectable bacterial species of the preparation, wherein Lactobacillus iners comprises at least 70% and Lactobacillus gasseri comprises less than 5% of all detectable bacterial species of the preparation. In some embodiments, the preparation comprises Lactobacillus iners and Lactobacillus gasseri, which together comprise about 80-99.9% of all detectable bacterial species of the preparation, wherein Lactobacillus iners comprises at least 80% and Lactobacillus gasseri comprises less than 5% of all detectable bacterial species of the preparation. In some embodiments, the preparation comprises Lactobacillus iners and Lactobacillus gasseri, which together comprise about 80-99.9% of all detectable bacterial species of the preparation, wherein Lactobacillus iners comprises at least 90% and Lactobacillus gasseri comprises less than 5% of all detectable bacterial species of the preparation.

In one aspect, the substantially complete vaginal microbiota preparation of the invention comprises Lactobacillus iners, Lactobacillus crispatus and Lactobacillus jensenii, which together comprise about 80-99.9% of all detectable bacterial species of the preparation; and less than 5% of Gardnerella spp., Atopobium spp., and Prevotella spp. In some embodiments, the preparation does not comprise detectable quantities of Lactobacillus gasseri. In some embodiments, the preparation comprises Lactobacillus iners, Lactobacillus crispatus and Lactobacillus jensenii, which together comprise about 80-99.9% of all detectable bacterial species of the preparation, wherein each of Lactobacillus crispatus and Lactobacillus jensenii comprises at least 0.01 to about 10% of all detectable bacterial species of the preparation. In some embodiments, the preparation comprises Lactobacillus crispatus, Lactobacillus crispatus and Lactobacillus jensenii, which together comprise about 80-99.9% of all detectable bacterial species of the preparation, wherein each of Lactobacillus crispatus and Lactobacillus jensenii comprises at least 0.01 to about 5% of all detectable bacterial species of the preparation. In some embodiments, the preparation comprises Lactobacillus iners, Lactobacillus crispatus and Lactobacillus jensenii, which together comprise about 80-99.9% of all detectable bacterial species of the preparation, wherein each of Lactobacillus crispatus and Lactobacillus jensenii comprises about 0.01-1% of all detectable bacterial species of the preparation. In some embodiments, the preparation comprises Lactobacillus iners, Lactobacillus crispatus and Lactobacillus jensenii, which together comprise about 80-99.9% of all detectable bacterial species of the preparation, wherein Lactobacillus jensenii comprises less than 2%, 1%, 0.5%, 0.2% of all detectable bacterial species of the preparation. In some embodiments, the preparation comprises Lactobacillus iners, Lactobacillus crispatus and Lactobacillus jensenii, which together comprise about 80-99.9% of all detectable bacterial species of the preparation, wherein Lactobacillus crispatus comprises less than 2%, 1%, 0.5%, 0.2% of all detectable bacterial species of the preparation. In some embodiments, the preparation comprises Lactobacillus iners, Lactobacillus crispatus and Lactobacillus jensenii, which together comprise about 80-99.9% of all detectable bacterial species of the preparation, wherein each of Lactobacillus iners and Lactobacillus jensenii comprises less than 2%, 1%, 0.5%, 0.2% of all detectable bacterial species in the preparation.

In one aspect, the substantially complete vaginal microbiota preparation of the invention comprises Lactobacillus iners, Lactobacillus crispatus and Lactobacillus gasseri, which together comprise about 80-99.9% of all detectable bacterial species of the preparation; and less than 5% of Gardnerella spp., Atopobium spp., and Prevotella spp. In some embodiments, the preparation does not comprise detectable quantities of Lactobacillus jensenii. In some embodiments, the preparation comprises Lactobacillus iners, Lactobacillus crispatus and Lactobacillus gasseri, which together comprise about 80-99.9% of all detectable bacterial species of the preparation, wherein each of Lactobacillus crispatus and Lactobacillus gasseri comprises at least 0.01 to about 10% of all detectable bacterial species of the preparation. In some embodiments, the preparation comprises Lactobacillus iners, Lactobacillus crispatus and Lactobacillus gasseri, which together comprise about 80-99.9% of all detectable bacterial species of the preparation, wherein each of Lactobacillus crispatus and Lactobacillus gasseri comprises at least 0.01 to about 5% of all detectable bacterial species of the preparation. In some embodiments, the preparation comprises Lactobacillus iners, Lactobacillus crispatus and Lactobacillus gasseri, which together comprise about 80-99.9% of all detectable bacterial species of the preparation, wherein each of Lactobacillus crispatus and Lactobacillus gasseri comprises about 0.01-1% of all detectable bacterial species of the preparation. In some embodiments, the preparation comprises Lactobacillus iners, Lactobacillus crispatus and Lactobacillus gasseri, which together comprise about 80-99.9% of all detectable bacterial species of the preparation, wherein Lactobacillus gasseri comprises less than 2%, 1%, 0.5%, 0.2% of all detectable bacterial species of the preparation. In some embodiments, the preparation comprises Lactobacillus iners, Lactobacillus crispatus and Lactobacillus gasseri, which together comprise about 80-99.9% of all detectable bacterial species of the preparation, wherein Lactobacillus crispatus comprises less than 2%, 1%, 0.5%, 0.2% of all detectable bacterial species of the preparation. In some embodiments, the preparation comprises Lactobacillus iners, Lactobacillus crispatus and Lactobacillus gasseri, which together comprise about 80-99.9% of all detectable bacterial species of the preparation, wherein each of Lactobacillus iners and Lactobacillus gasseri comprises less than 2%, 1%, 0.5%, 0.2% of all detectable bacterial species in the preparation.

In one aspect, the substantially complete vaginal microbiota preparation of the invention comprises Lactobacillus crispatus, Lactobacillus iners, Lactobacillus jensenii and Lactobacillus gasseri, which together comprise about 80-99.9% of all detectable bacterial species of the preparation; and less than 5% of Gardnerella spp., Atopobium spp., and Prevotella spp. In some embodiments, the preparation comprises Lactobacillus crispatus, Lactobacillus iners, Lactobacillus jensenii and Lactobacillus gasseri, which together comprise about 80-99.9% of all detectable bacterial species of the preparation, wherein Lactobacillus iners comprises at least 50%, 60%, 70% or 80% of all detectable bacterial species of the preparation. In some embodiments, the preparation comprises Lactobacillus crispatus, Lactobacillus iners, Lactobacillus jensenii and Lactobacillus gasseri, which together comprise about 80-99.9% of all detectable bacterial species of the preparation, wherein each of Lactobacillus jensenii, Lactobacillus iners and Lactobacillus gasseri comprises 0.01-25%, 0.1-20% or 0.2-15% of all detectable bacterial species of the preparation. In some embodiments, the preparation comprises Lactobacillus crispatus, Lactobacillus iners, Lactobacillus jensenii and Lactobacillus gasseri, which together comprise about 80-99.9% of all detectable bacterial species of the preparation, wherein each of Lactobacillus jensenii, Lactobacillus crispatus and Lactobacillus gasseri comprises 0.01 to about 5% of all detectable bacterial species of the preparation. In some embodiments, the preparation comprises Lactobacillus crispatus, Lactobacillus iners, Lactobacillus jensenii and Lactobacillus gasseri, which together comprise about 80-99.9% of all detectable bacterial species of the preparation, wherein Lactobacillus iners comprises at least 50% of all detectable bacterial species of the preparation, and further wherein each of Lactobacillus jensenii, Lactobacillus crispatus and Lactobacillus gasseri comprises 0.01 to about 25% of all detectable bacterial species of the preparation.

Lactobacillus jensenii-Dominant Preparations

The invention further provides Lactobacillus jensenii-dominant substantially complete vaginal microbiome preparations, wherein Lactobacillus jensenii is present in a greater amount than each of Lactobacillus crispatus, Lactobacillus iners and Lactobacillus gasseri. Preferred aspects and embodiments of Lactobacillus jensenii-dominant substantially complete vaginal microbiome preparations are provided in the following.

In one aspect, the substantially complete vaginal microbiota preparation of the invention comprises Lactobacillus jensenii, Lactobacillus crispatus, Lactobacillus iners and Lactobacillus gasseri in a relative quantity of about 80-99.9% of all detectable bacterial species of the preparation; and less than 5% of Gardnerella spp., Atopobium spp., and Prevotella spp. In some embodiments, preparation comprises Lactobacillus jensenii in a relative quantity of above 60%, 65%, 70% or 75% of all detectable bacterial species of the preparation. In some embodiments, each of Lactobacillus crispatus, Lactobacillus iners, Lactobacillus gasseri comprises 0.01 to about 20% of all detectable bacterial species of the preparation. In some embodiments, each of Lactobacillus crispatus, Lactobacillus iners, Lactobacillus gasseri comprises 0.05 to about 15% of all detectable bacterial species of the preparation. In some embodiments, Lactobacillus jensenii comprises at least 65% of all detectable bacterial species of the preparation, and further wherein each of Lactobacillus crispatus, Lactobacillus iners, Lactobacillus gasseri comprises 0.01 to about 15% of all detectable bacterial species of the preparation.

Lactobacillus gasseri-Dominant Preparations

The invention further provides Lactobacillus gasseri-dominant substantially complete vaginal microbiome preparations, wherein Lactobacillus gasseri is present in a greater amount than each of Lactobacillus crispatus, Lactobacillus jensenii and Lactobacillus gasseri. Preferred aspects and embodiments of Lactobacillus gasseri-dominant substantially complete vaginal microbiome preparations are provided in the following.

In one aspect, the substantially complete vaginal microbiota preparation of the invention comprises Lactobacillus gasseri, in a relative quantity of about 80-99.9% of all detectable bacterial species of the preparation; and less than 5% of Gardnerella spp., Atopobium spp., and Prevotella spp. In some embodiments, preparation comprises Lactobacillus gasseri, in a relative quantity of above 90%, 95%, 99% or 99.9% of all detectable bacterial species of the preparation. In some embodiments, the species Lactobacillus crispatus, Lactobacillus jensenii, Lactobacillus gasseri are not present in detectable quantities in the preparation.

In another aspect, the substantially complete vaginal microbiota preparation of the invention comprises Lactobacillus gasseri, Lactobacillus crispatus, Lactobacillus iners and/or Lactobacillus jensenii, which together comprise about 80-99.9% of all detectable bacterial species of the preparation; and less than 5% of Gardnerella spp., Atopobium spp., and Prevotella spp. In some embodiments, the preparation does not comprise detectable quantities of Lactobacillus iners and/or Lactobacillus jensenii. In some embodiments, the preparation does not comprise detectable quantities of Lactobacillus crispatus and/or Lactobacillus jensenii. In some embodiments, the preparation does not comprise detectable quantities of Lactobacillus iners and/or Lactobacillus crispatus. In some embodiments, the preparation comprises Lactobacillus gasseri in a relative quantity of at least 40%, 45%, 50%, 55%, 60%, 65% or more of all detectable bacterial species of the preparation.

The substantially complete vaginal microbiota preparation, and pharmaceutical composition comprising the same, are suitable for administration, preferably vaginal administration, to a subject. The subject can be a human.

In one aspect, the substantially complete vaginal microbiota preparation provided herein is suitable for vaginal administration. In a further aspect, the substantially complete vaginal microbiota preparation provided herein is for use in treating inflammation or a disorder associated with inflammation. In a further aspect, the substantially complete vaginal microbiota preparation provided herein comprises an effective amount of vaginal lactobacilli for engrafting in a recipient's vaginal niche and treating inflammation.

Lactobacilli in the Vaginal Niche

Lactobacilli are the predominant microorganisms in the vaginal microbial community and they play a major role in maintaining a healthy urogenital tract. Lactobacilli are capable of preventing adhesion and growth of pathogenic microorganisms and/or overgrowth of pathobionts through mechanisms that appear to involve secretion of anti-adhesion factors, hydrogen peroxide, bacteriocins and fermenting the glycogen to lactic acid, thereby creating an acidic environment hostile to pathogens and pathobionts. The genus Lactobacillus comprises a phenotypically heterogenous group of Gram-positive, aerotolerant anaerobic, lactic acid producing bacteria. Other typical characteristics include being catalase-negative and rod-shaped, and generally possess DNA with a low content of guanine (G) and cytosine (C), less than about 50%. They are members of the phylum Firmicutes, class Bacilli, order Lactobacillales and family Lactobacillaceae. One skilled in the art will be able to identify Lactobacillus species using standard techniques.

Species of lactobacilli can be identified phenotypically, as well as genetically, e.g., on the basis of 16S rRNA (ribosomal RNA) sequence (or the DNA encoding the 16S rRNA, generally referred to as 16S rDNA). Genetic analysis can be performed using standard techniques, for example whole genome sequencing analysis as well as widely used typing approaches based on nucleotide variation in several hundred DNA sequences and a few gene fragments: Multi-locus Sequence Typing (MLST), Multi-locus Variable number of tandem repeats Analysis (MLVA), rMLST and cgMLST) discussed, e.g., in Marcos Pérez-Losada, M. et al., “Microbial sequence typing in the genomic era”, Infection, Genetics and Evolution, Vol. 63, September 2018, p. 346-359.

Other identification techniques include: Vaginal pH, Nugent Score, Whiff Test, gas liquid chromatographic analysis of glucose fermentation products, total anaerobe concentrations, total aerobe concentrations, enzymatic activity (e.g., lipase, phospholipase A2 and phospholipase C, hydrogen peroxide production).

In some embodiments, the substantially complete vaginal microbiota preparations described herein do not comprise (and are not derived from) isolated and/or culture-propagated bacterial strain(s). Substantially complete vaginal microbiota preparations can be prepared from cervicovaginal secretions (vaginal fluid), e.g., collected from the vaginal tract of a female donor with a healthy vaginal flora. Substantially complete vaginal microbiota preparations can be collected using standard techniques using commercially available collection devices, such as, a menstrual fluid collection device (soft cup or soft disc), a syringe, a tube, spatula or beaker, or an absorbent matrix. In some embodiments, the menstrual fluid collection device is a vaginal self-sampling device. A vaginal self-sampling device can be, e.g., used by donors to collect vaginal fluid or cervicovaginal secretions without the help of another person.

Optionally, the collected material is undergoing centrifugation. The centrifugation step may be performed to facilitate collection, without physical separation of vaginal fluid components. In some embodiments, the substantially complete vaginal microbiota preparations comprise one or more of: mucus (e.g., secreted by the cervix), shed epithelial cells, vaginal transudate, and bacteria other than lactobacilli found in the secretion from the female donor. It is thought that mucus and other components of the secretion (including other bacteria) are beneficial to Lactobacillus growth and survival upon administration to the urogenital tract thereby supporting engraftment in a female recipient. This provides an advantage over compositions comprising isolated strain(s). Thus, in some embodiments, the substantially complete vaginal microbiota preparation is not cultured (e.g., for the purpose of strain isolation) or propagated in vitro but rather preferably stored in the refrigerator at 4° C. or immediately frozen after collection (e.g., frozen to 0° C., −20° C., −80° C., −190° C.), or optionally spray dried or lyophilized.

If desired, the cervicovaginal secretions can be further processed, e.g., prior to refrigeration or freezing, e.g., by filtration for sterility and/or to remove residual particles, aggregates and cells, and adding diluent, e.g., to arrive at a desired volume, concentration (e.g., CFU/mL) and/or viscosity, as discussed herein. Optionally, the substantially complete vaginal microbiota preparation is kept refrigerated or frozen until it is formulated into a dosage form and/or dispensed into an applicator or dispenser.

Other advantages of the substantially complete vaginal microbiota preparations described herein include that, in embodiments, substantial engraftment does not require the use of mucus adhesive excipients (such as, e.g., hydrocolloids, e.g., xanthan gum, locust bean gum alginate) to increase the ability to colonize by adherence of lactobacilli to the mucosal membrane, which is sometimes used to increase engraftment of isolated, propagated strains. This minimizes the risk of triggering an adverse side effect, such as an allergic reaction due to the mucus adhesive excipient, and increases tolerance. Another advantage of the substantially complete vaginal microbiota preparations described herein is the efficient and prolonged engrafting/colonization of the lactobacilli in the vaginal mucosa. In some embodiments, the colonization effect persists for at least 1, 2, 3, 4, 5 or more menstrual cycles after administration of the substantially complete vaginal microbiota preparation.

One further advantage of the inventive method and preparation lies in that no pre-treatment with antibiotics is required to successfully treat inflammation of the dysbiotic subject, which was not previously possible. This is likely due to the smaller sample concentrations or use of single strains, whereas the preparation provided herein comprises not only the entire ecosystem of the vaginal microbiota but also a higher concentration and/or amount of the vaginal microbiota in the preparation. Lastly, the liberal use or overuse of antibiotics is directly associated with the problem of increasing antibiotic resistance. The present preparation is not reliant on using antibiotics prior to or during treatment, which is thus a further advancement and advantageous effect of the present invention.

Pioneer Species

Another advantage of the substantially complete vaginal microbiota preparation is the presence of a substantially complete ecosystem of the donor's vaginal microbiota. Even lactobacilli that are present in very low quantities, such as 0.01% or even 0.001%, in certain embodiments, are successfully transferred to the recipient by administering the preparation. Without being bound by theory, it is believed that a substantially complete ecosystem provides optimal growth conditions for pioneer species, such as, e.g., Lactobacillus jensenii, Lactobacillus gasseri and/or Lactobacillus iners, which preferentially colonize and proliferate in the sub-optimal recipient dysbiotic vaginal niche, e.g., at higher pH.

In embodiments, the pioneer species are characterized by being able to initially engraft and proliferate in the dysbiotic vaginal niche, thereby, e.g., liberating metabolites (including amino acids) and lowering the pH. This in turn would be expected to promote the proliferation of other Lactobacilli present in the preparation and vaginal cavity.

By providing a substantially complete ecosystem, the vaginal Lactobacillus species comprised in the substantially complete vaginal microbiota preparation may act in concert to reverse the dysbiosis and result, in certain embodiments, in ‘twinning’ of the vaginal microbiome, e.g., reproducing a near copy of the donor's healthy vaginal microbiota in the recipient's vaginal niche.

It is thought that Lactobacillus species have species-specific optimal pH ranges at which they proliferate and thrive. L. crispatus typically proliferates well in a pH range of about 3.7-4.0, more optimally at pH 3.7-3.9. L. iners typically proliferates best at a pH range of 3.9-4.2. L. gasseri and L. jensenii proliferate best at pH values of about 4.0-5.2. Particularly L. jensenii can proliferate well at high pH.

In some embodiments, the substantially complete vaginal microbiota preparation comprises one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the preparation, wherein the L. crispatus is present at a relative concentration of at least 50%, at least 60%, at least 70%, at least 80%, at least 90%, or about 99.9%, further wherein the rate of engraftment and/or proliferation of L. jensenii after administration is higher relative to the rate of engraftment and/or proliferation of L. crispatus.

In some embodiments, the substantially complete vaginal microbiota preparation comprises one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the preparation, wherein the L. crispatus and L. iners are both present at about 30-45%, further wherein the rate of engraftment and/or proliferation of L. jensenii after administration is higher relative to the rate of engraftment and/or proliferation of L. crispatus.

In some embodiments, the substantially complete vaginal microbiota preparation comprises one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the preparation, wherein the L. crispatus and L. iners are both present at about 30-45%, further wherein the rate of engraftment and/or proliferation of L. gasseri after administration is higher relative to the rate of engraftment and/or proliferation of L. crispatus.

In some embodiments, the substantially complete vaginal microbiota preparation comprises one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the preparation, wherein the L. crispatus and L. iners are both present at about 30-45%, further wherein the rate of engraftment and/or proliferation of L. iners after administration is higher relative to the rate of engraftment and/or proliferation of L. crispatus.

In some embodiments, substantially complete vaginal microbiota preparations comprise L. iners, L. gasseri and/or L. jensenii species, capable of proliferating in a pH from about 3.8-5.0, 4.0-5.0, 4.0-4.8, 4.0-4.6, 4.0-4.5, 4.2-5.0, 4.2-4.8, 4.2-4.6, 4.4-4.8 or pH 4.4-5.0. In some embodiments, the proliferation of L. iners, L. gasseri and/or L. jensenii reduces the pH in the recipient vaginal niche to a pH hospitable for proliferation of L. crispatus. In some embodiments, the proliferation of L. iners, L. gasseri and/or L. jensenii reduces the pH in the recipient vaginal niche to a pH suitable for proliferation of L. crispatus. In some embodiments, after administration of the preparation, the at least one of L. iners, L. gasseri and/or L. jensenii has the highest rate of engraftment and/or proliferation, which is greater than the rate of proliferation of L. crispatus.

In embodiments, the successful administration and treatment of dysbiosis using the substantially complete vaginal microbiota preparation transfers the vaginal ecosystem of the donor to the recipient, including minor species that might be present at low relative quantities.

After administration of the substantially complete vaginal microbiota preparation to the dysbiotic recipient, the pioneer species, e.g., L. iners, L. gasseri and/or L. jensenii, may have a proliferative advantage, e.g., due to their specific tolerability of a higher pH range, which is compatible with the resident pH in the dysbiotic vaginal niche. The Lactobacillus species comprised in the ecosystem may thus not colonize and proliferate at the same rate after administration, but depending on, e.g., the pH of the dysbiotic vaginal niche may favor a subset of the lactobacilli, such as, e.g., the pioneer species of the ecosystem (e.g., L. iners, L. gasseri and/or L. jensenii), to proliferate first. This initial proliferation of the pioneer species renders the dysbiotic vaginal niche habitable for the “follower-species”, such as, e.g., L. crispatus, which prefer a lower pH to thrive. The proliferation of the pioneer species, e.g., L. iners, L. gasseri and/or L. jensenii, may help to reduce pH level, e.g., from pH 4.8 to 4.0, thereby creating more amenable conditions for the proliferation of L. crispatus.

It is predicted that the interplay between the different Lactobacillus species present in the substantially complete vaginal microbiota preparation affect the successful treatment of dysbiosis. This advantageous effect would not be observable with isolated strains or isolated species.

In some embodiments, the lactobacilli comprised in the substantially complete vaginal microbiota preparations described herein are capable of colonizing and becoming established (engrafted) in a human vagina. In some embodiments, colonization and engraftment occurs even during menstrual discharge. The lactobacilli comprised in the substantially complete vaginal microbiota preparations may continue to reside in the vagina after administration over several menstruation cycles. In some embodiments, the lactobacilli engraft in the vaginal microbial niche over one or more than one (e.g., two, three, four, five or six) menstruation cycle upon vaginal administration. Residence time can be determined, e.g., using nucleic acid sequencing, e.g., for specific lactobacilli, e.g., comparing sequencing results prior to and post administration of the substantially complete vaginal microbiota preparations described herein, e.g., to determine the identity of newly added members (e.g., one or more lactobacilli) to the recipient's microbial community, and then at predetermined time intervals (e.g., prior or post a menstruation cycle, optionally, over a number of menstruation cycles) determine (e.g., through nucleic acid sequencing) that all or a certain subset of the newly added members are still substantially present at the specific time interval. In some embodiments, the residence time of the recipient's microbiome may be compared to the donor's microbiome. The residence time may vary depending on various factors including hormone levels (e.g., estrogen), diet, sexual activity, acidity status of the vagina, and the presence or absence of genital infections and other microbial perturbations, e.g., treatment with antibiotics.

Successful colonization and engraftment of the lactobacilli comprised in the substantially complete vaginal microbiota preparations may be indicated by one or more of: decreased pH, increased lactic acid content, lower abundance of antibiotic resistance genes, decreased amount of fungal DNA, decreased toxin content, decreased pathogenicity factors, decreased inflammatory cytokines and chemokines, decreased immune cell infiltrates, decreased total bacterial DNA load, decreased total pathogenic DNA load, increased viscoelasticity, increased sialoglycan content, decreased relative or absolute abundance of pathobionts or pathogens, or any combination thereof in the vaginal cavity and the vaginal microbial niche (e.g., when compared to baseline of the same subject (e.g., prior to administration and engraftment) or a non-treated control subject).

Stability

The lactobacilli comprised in the substantially complete vaginal microbiota preparations or in pharmaceutical compositions formulated with the substantially complete vaginal microbiota preparations described herein are generally viable for up to one week if stored in the refrigerator at 4° C. and up to several months (e.g., 1, 2, 3, 4, 5, 6, 9, 12, 15, 18, or 24 months, or longer) if stored frozen at about −18° C., or preferably at about −80° C. Viability decreases over time. The substantially complete vaginal microbiota preparations or pharmaceutical compositions thereof described herein are suitable for administration if they retain at least 30%, 40%, 50%, 60%, 70%, or at least 80% viable bacteria prior to use. Viable cells are generally able to colonize and engraft. Percent viability refers to the percentage of viable bacteria in a population. In some embodiments, the substantially complete vaginal microbiota preparations described herein are further processed to add a diluent, such as, e.g., saline, e.g., to formulate a pharmaceutical composition. In some embodiments, the substantially complete vaginal microbiota preparations or compositions thereof described herein are further processed to be freeze-dried (lyophilized), e.g., for easy storage, packaging, formulated in, for instance, compositions, and transport, and can be rehydrated before administration.

Some Composition Characteristics

Provided herein are compositions, such as pharmaceutical compositions comprising the substantially complete vaginal microbiota preparations. In some embodiments, the pharmaceutical compositions comprise i) a substantially complete vaginal microbiota preparation comprising, e.g., one or more Lactobacillus species and optionally one or more residual constituents of a cervicovaginal secretion (such as, e.g., vaginal mucus, vaginal transudate, other vaginal fluids, and vaginal epithelial cells), ii) a pharmaceutically acceptable buffer or diluent or other excipients, such as, e.g., saline (e.g., to adjust the viscosity and/or isotonicity/osmolarity of the composition), iii) one or more acidifying agents, e.g., lactic acid or boric acid (e.g., to adjust the pH of the composition, e.g., to below pH 5, or between pH 3.0 and 4.5), iv) one or more other active agents, such as, e.g., prebiotics (e.g., to generate a synbiotic mixture with the substantially complete vaginal microbiota preparation), spermicides (e.g., to kill or inactivate residual sperm in the preparation); hormonal agents (e.g., estrogen), anti-inflammatory agents, and the like. In preferred embodiments, the pharmaceutical compositions comprise (i) and (ii). In one embodiment, the pharmaceutical compositions consist of (i) and (ii). In one embodiment, the pharmaceutical compositions comprise (i) and (ii) but not (iii) and (iv). In one embodiment, the pharmaceutical compositions comprise (i), (ii), and (iii) but not (iv). In one embodiment, the pharmaceutical compositions comprise (i), (ii), (iii) and a spermicide. In some embodiment, the pharmaceutical compositions do not comprise prebiotics, hormonal or anti-inflammatory agents.

Acidifying Agents

The compositions comprising a substantially complete vaginal microbiota preparation described herein may optionally comprise one or more acidifying agents, such as, e.g., organic acids or salts thereof. In some embodiments, acidifying agents may be used to reduce the pH of the compositions, e.g., to below pH 5.5 or 5.0, e.g., to between pH 3.5 to 4.5, or pH 3.0 to 4.5. In decreasing pH such acidifiers may act as anti-microbial agents (e.g., to inhibit Candida or pathogenic bacteria). Acidifying agents comprise, e.g., lactic, acetic, ascorbic, citric, folic sorbic, or boric acid. In other embodiments, the acidifying agent is administered separate from the compositions comprising a substantially complete vaginal microbiota preparation; e.g. prior to, concurrent with, or after administration of the composition (e.g., in a different dosage form, such as, e.g. a suppository, cream, gel, powder, douche or similar), e.g., for one to seven days, or one to ten days. In these embodiments, the acidifying agent may be used to promote the survival and engraftment of the lactobacilli comprised in the substantially complete vaginal microbiota preparation. Acidifying agents may also be useful as spermicides, e.g., to kill or inactivate residual sperm that may be present in the preparation. In one embodiment, the spermicidal activity is contributed by adding lactic acid. In one embodiment, lactic acid may be provided as a racemic mixture of D- and L-isomers, or at different suitable ratio, including, e.g., only D-lactate or only L-lactate.

In some embodiments, the compositions comprising a substantially complete vaginal microbiota preparation is acidified by adding 0.1%, 0.5%, 1%, 1.5%, 2%, 2.5%, 3% or 5% of acidifier (e.g., lactic acid). In preferred embodiments, the acidifier is added at a concentration of 0.5% to 1.5%, or 0.2% to 2% (w/w), or a similar concentration that matches a healthy vagina.

Viscosity

The compositions comprising a substantially complete vaginal microbiota preparation described herein may comprise one or more pharmaceutically acceptable buffer or diluent or other excipients. In preferred embodiments, the diluent is saline (e.g., 0.9% NaCl), e.g., sterile normal saline. In one embodiment, the diluent is used to adjust the viscosity of the composition, e.g., to lower the viscosity of the substantially complete vaginal microbiota preparation derived from the cervicovaginal secretion, thereby increasing the ability to administer the composition to a female recipient, e.g., using an applicator or dispenser or similar vaginal delivery system. In other embodiments, one or more excipients is used to formulate the composition in different dosage forms, such as, e.g., suppositories, creams or dissolving films or tablets.

In some embodiments, the dosage form is liquid, solid or semi-solid. The solid dosage form preferably comprises a tablet, capsule, or a film. The semi-solid dosage form preferably comprises a suppository, ointment, gel, cream or rigid foam. In a preferred embodiment, the dosage form is a gel.

When adjusting the viscosity for administration to a female recipient's vaginal cavity, e.g., using an applicator or dispenser, care should be taken to adjust the viscosity to be easily dispensable in the vaginal cavity (e.g., without having to apply significant force to expel the liquid composition), yet avoid making the composition so fluid/liquid that is does not stay in the vaginal cavity for any appreciable amount of time after administration. Thus, a viscosity should be selected that is suitable to prevent rapid discharge from the vaginal cavity. The compositions comprising a substantially complete vaginal microbiota preparation described herein are preferably of a viscosity which allows the majority of it to stay in the vagina for at least 5 minutes, at least 10 minutes, at least 20 minutes, at least 30 minutes, when the female recipient is in an upright position, although administration is preferably carried out in a lithotomy position, e.g., with the female recipient lying down. Viscosity can be measured, e.g., using a Viscometer.

Spermicides

The compositions comprising a substantially complete vaginal microbiota preparation described herein may comprise one or more spermicides. Care should be taken when using nonoxynol-9 (N-9) because of its membrane disruptive properties (it increases the permeability of vaginal tissue by causing damage the cervicovaginal epithelium) and has been shown to be detrimental to Lactobacillus spp. Other spermicides include octoxynol-9, sodium cholate, and benzalkonium chloride. In one embodiment, lactic acid is used. In other embodiments, lactic acid may be used in combination, e.g., with citric acid.

Prebiotics

A “prebiotic” as used herein is a growth substrate, which increases growth of bacteria (such as lactobacilli) comprised in the substantially complete vaginal microbiota preparations, as could be measured, e.g., in vitro. If desired, though it is not generally necessary, a prebiotic may be added to the compositions described herein, e.g., to create a synbiotic mixture, e.g., to increase growth of the bacteria comprised in the substantially complete vaginal microbiota preparations upon administration to the genitourinary tract of a female recipient. This may, under certain circumstances, increase successful colonization and engraftment. In other embodiments, prebiotics may be used for the maintenance of an engrafted preparation and/or general vaginal health.

If a prebiotic is desired, it should be carefully chosen to not be greatly metabolizable by any yeast (e.g., Candida species), pathobionts or pathogens (e.g., E. coli and other Gram-negative bacteria) that may reside in the female recipient's genitourinary tract (e.g., to avoid promoting their growth). Prebiotics include, e.g., lactitol, lactulose, and in some instances also other oligosaccharides and soluble fibers, e.g., fructooligosaccharides (FOS), glucooligosaccharides (GOS), and inulin.

Other Active Agents

If desired, other active agents may be added to the compositions described herein, e.g., to address bacterial or fungal infections and/or sexually transmitted diseases in the recipient female, for example antimicrobial agents, antifungal agents, antibacterial agents, antiviral agents, antibiotics, antiparasitic agents (e.g., with activities against Trichomonas vaginalis), anti-inflammatory agents, and the like. Care must be taken when formulating these agents into the pharmaceutical composition so as to not substantially interfere with the activity and efficacy of the substantially complete vaginal microbiota preparations (and lactobacilli) comprised in the compositions.

In some embodiments, the pharmaceutical compositions comprise a form of estrogen. Adequate levels of estrogens play a role in the trophism of vaginal mucosa, and estrogens increase the cellular content of glycogen.

In some embodiments, the pharmaceutical compositions comprise thiosulfate, e.g., to potentiate the anti-pathogenic effect of lactobacilli.

If desired, the pharmaceutical compositions can further contain an antibiotic, such as, e.g., metronidazole, or one or more antibiotics of the following classes: a macrolide (e.g., azithromycin, clarithromycin and erythromycin), a tetracycline (e.g., doxycycline, tigecycline), a fluoroquinolone (e.g., gemifloxacin, levofloxacin, ciprofloxacin and mocifloxacin), a cephalosporin (e.g., ceftriaxone, defotaxime, ceftazidime, cefepime), a penicillin (e.g., amoxicillin, amoxicillin with clavulanate, ampicillin, piperacillin, and ticarcillin) optionally with a beta-lactamase inhibitor (e.g., sulbactam, tazobactam and clavulanic acid), such as ampicillin-sulbactam, piperacillin-tazobactam and ticarcillin with clavulanate, an aminoglycoside (e.g., amikacin, arbekacin, gentamicin, kanamycin, neomycin, netilmicin, paromomycin, rhodostreptomycin, streptomycin, tobramycin, and apramycin), a penem or carbapenem (e.g. doripenem, ertapenem, imipenem and meropenem), a monobactam (e.g., aztreonam), an oxazolidinone (e.g., linezolid), vancomycin, glycopeptide antibiotics (e.g. telavancin), and the like.

In some embodiments, the pharmaceutical compositions can further contain an antimicrobial (an antibiotic or antifungal) selected from metronidazole, tinidazole, secnidazole, clindamycin, nystatin, azithromycin, erythromycin, ofloxacin, doxycycline, levofloxacin, amoxicillin, and fluconazole.

If desired, the pharmaceutical composition can contain an agent for treating infections with mycobacteria. Suitable agents for treating infections with mycobacteria include an aminoglycoside (e.g., capreomycin, kanamycin, streptomycin), a fluoroquinolone (e.g. ciprofloxacin, levofloxacin, moxifloxacin), isozianid and isozianid analogs (e.g. ethionamide), aminosalicylate, cycloserine, diarylquinoline, ethambutol, pyrazinamide, protionamide, rifampin, and the like.

If desired, the pharmaceutical composition can contain a suitable antiviral agent, such as remdesivir, oseltamivir, zanamavir, amantidine or rimantadine, ribavirin, gancyclovir, valgancyclovir, foscavir, Cytogam® (cytomegalovirus immune globulin), pleconaril, rupintrivir, palivizumab, motavizumab, cytarabine, docosanol, denotivir, cidofovir, and acyclovir.

If desired, the pharmaceutical composition can contain a suitable antifungal agent, such as polyene (e.g., nystatin and natamycin) and imidazole antifungals (e.g., flucanozole and clotrimazole).

If desired, the pharmaceutical composition can contain one or more suitable steroids. For example, the composition may include androgens/anabolic steroids, estrogens, progestogens, corticosteroids, neurosteroids, estradiol, estropipate, premarin, drospirenone, noresthisterone, levonorgestrel, testosterone, fluoxymesterone, methylesterosterone, oxandrolone, and oxymetholone.

Combination Therapies

Provided herein are also methods to treat a subject, e.g., a human female, in need thereof, e.g., a subject exhibiting dysbiosis (e.g., dysbiosis in the urogenital tract) by administering an effective amount of a composition, such as a pharmaceutical composition comprising a substantially complete vaginal microbiota preparation described herein.

It will be understood by one of skill in the art that the administration regimen for treating may include one or more other therapies in combination with the administration of the substantially complete vaginal microbiota preparations and compositions described herein. In some embodiments, the methods for treating vaginal dysbiosis described herein may further comprise administering standard of care treatment. For example, the methods for treating vaginal dysbiosis described herein may further comprise administering antimicrobial agents, antifungal agents, antibacterial agents, antiviral agents, antibiotics, antiparasitic agents (e.g., with activities against Trichomonas vaginalis), anti-inflammatory agents, and the like. Current standard of care antimicrobials (antibiotics, antifungals) include metronidazole, tinidazole, secnidazole, clindamycin, nystatin, azithromycin, erythromycin, ofloxacin, doxycycline, levofloxacin, amoxicillin, and fluconazole. In another example, the methods for treating vaginal dysbiosis described herein may further comprise administering thiosulfate, e.g., to help recolonize the vaginal microbiota and to prevent the regrowth of pathogenic agents and thus recurrences.

Any of the therapeutic agents described herein can be considered a) for formulation as a pharmaceutical composition with the substantially complete vaginal microbiota preparations, or b) as a combination therapy. These combination therapies may be administered together or separately from and concurrent with (substantially the same time) or sequentially to (e.g., prior to or after) administration of the substantially complete vaginal microbiota preparations and compositions described herein. They may be administered using different routes of administration and dosage forms, such as orally (e.g., as a pill) or topical (e.g., as a gel). The use combination therapies should be assessed to determine that the treatment does not substantially interfere with the activity and efficacy of the substantially complete vaginal microbiota preparations (and lactobacilli) comprised in the compositions and if they do, the regimen should be adjusted (e.g., timing, dosing, sequencing, etc.) to minimize the interference.

Vaginal Delivery Systems

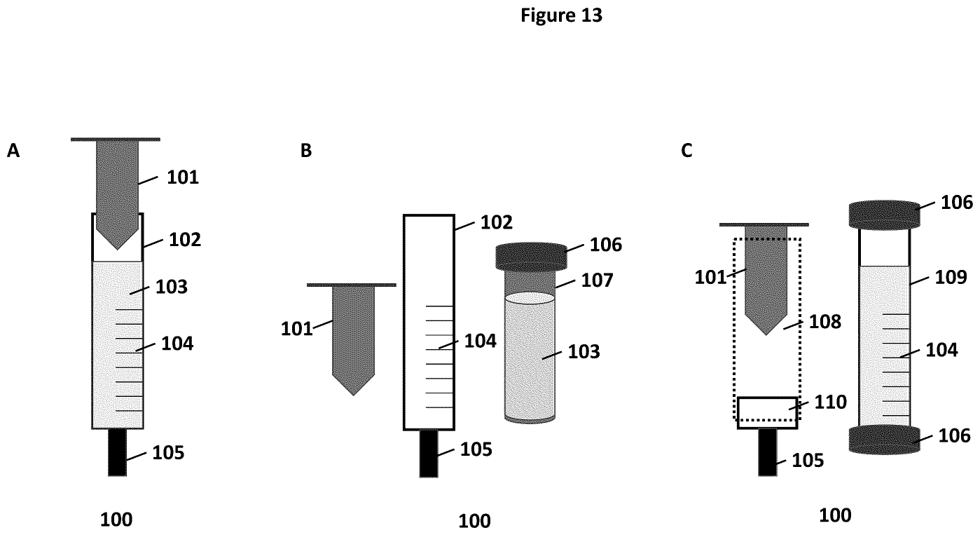

Provided herein are also vaginal delivery systems (e.g., FIGS. 13A, 13B, 13C).

Vaginal delivery systems may be used to administer a composition comprising a substantially complete vaginal microbiota preparation described herein, e.g., to a female (e.g., to the vaginal cavity). The vaginal delivery system provides means for administering the composition to the vaginal cavity. It may be comprised of a device or dosage form that is suitable for vaginal delivery of the compositions comprising a substantially complete vaginal microbiota preparation described herein. For example, a suitable device comprises an open end (e.g., a tip) for insertion into the vaginal cavity, and a dispensing end (e.g., a plunger or piston) to expel the composition through the open end. The compositions comprising a substantially complete vaginal microbiota preparation can be loaded through either end, and can be, e.g., packaged, either pre-filled or separate from the composition. If desired, the packaging is sterile. The device could then be used directly for administration (if pre-filled) or filled with the composition immediately (or shortly prior to) administration by a user. Alternatively, the device is filled with the composition and stored under refrigeration for a few days up to about a week (or alternatively frozen if it is stored for longer periods) prior to administration. In some cases, e.g., where the device is an applicator or dispenser, after release of the composition comprising a substantially complete vaginal microbiota preparation in the vaginal cavity, the device is removed from the vaginal cavity. Suitable devices include applicators and dispensers, e.g., those commonly used in medicine and science. The applicators and dispensers may either be operated manually or comprise electromechanical aides.

If desired, the vaginal delivery system is calibrated, e.g., has a volume indication or maximal volume indication or control, e.g., to aide dosing. In one embodiment, the vaginal delivery system is a single-use device, which is thrown away after each use. In another embodiment, the vaginal delivery system is a multiple-use device in which case it will have to be cleaned, and optionally sterilized, after each use. Both single use and multiple use applicators can be of any suitable material, such as, e.g., inert polymeric materials, such as polystyrene or polypropylene.

An exemplary and non-limiting embodiment of a vaginal delivery system is depicted in FIGS. 13A, 13B, and 13C with optional features.

In other embodiments, the vaginal delivery system comprises a vaginal suppository that will remain in the vaginal cavity until it is dissolved. Care needs to be taken when formulating a vaginal suppository or tablets to avoid damaging the bacteria (lactobacilli) comprised in the substantially complete vaginal microbiota preparation, e.g., when pressure or heat is applied during the formulation process that may inactivate the preparation. Measures should be taken to reduce the impact of the heat and pressure on the microorganisms. In a further embodiment, vaginal delivery systems comprise an absorbent product comprising the composition comprising a substantially complete vaginal microbiota preparation such as, e.g., a feminine hygiene diaper, panty liners, sanitary napkin, tampon, panty guard or an incontinence guard.

The vaginal delivery systems described herein may optionally comprise composition comprising a substantially complete vaginal microbiota preparation that are in dry form, e.g., lyophilized or spray dried and then optionally formulated into a dosage form described herein, or reconstituted, e.g., with sterile water, a weak acidic solution, gel, or buffer prior to use. If the composition is not reconstituted, depending on the precise formulation of the dosage form, rehydration may also be achieved inside the vaginal cavity, e.g., aided by resident vaginal fluid. Methods of preserving viable bacteria by lyophilization can promote long-term preservation of the microorganism. One skilled in the art will be able to lyophilize bacteria using standard techniques.

In all of these embodiments, dyes, perfumes, pH buffering agents, drying or resuspending agents, or other standard materials for drug formulation can be incorporated into the compositions and devices.

In all of these embodiments, devices and compositions described herein may be packed in a suitable packaging, for example in a bottle, flacon, blister pack, cartridge, applicator or dispenser. In one embodiment, the vaginal delivery system comprises a dosage form comprising the pharmaceutical composition or the substantially complete vaginal microbiota preparation of the invention suitable for administering the composition or preparation to the vaginal cavity, wherein the dosage form is an applicator, dispenser or suppository. In a further embodiment, the pharmaceutical composition or the substantially complete vaginal microbiota preparation is in liquid or semi-solid form. A solid dosage form comprises a capsule, a tablet and a film. The semi-solid dosage form comprises a suppository, ointment, gel, cream or rigid foam.

Dosage Forms and Formulations

Multiple dosage forms comprising a substantially complete vaginal microbiota preparation suitable for the vaginal delivery systems are contemplated herein, and include a suspension, spray, gel, cream, ointment, powder, (gelatin or vegetable cellulose) capsule, solution for lavages or douches, foams, films, ovules, a vaginal insert (e.g. tampon), tablets, disk, wafer (e.g., drying on film, by vaporization), or a microencapsulated product employing excipients and formulation techniques known to those skilled in the art. Particularly preferred dosage forms include formed gels, lyophilized gels, tablets, frozen formulations and films

A number of suitable excipients can be used to formulate the substantially complete vaginal microbiota preparation, such as bulking agents, polymers, carbon sources, mucoadhesive agents, or pH modifiers and/or buffers.

The carbon source excipients may act as a carbon source for the microbiota contained in the substantially complete vaginal microbiota preparation. Such carbon courses comprise mannitol, maltodextran, and Guar gum. Some excipients may further serve as mucoadhesive agents or as viscosity agents. Bulking agents may comprise one or more of mannitol, micro-crystalline cellulose, maltodextran, guar gum, inulin, or alginic acid (e.g., sodium alginate). Polymers may comprise structural polymers. In some embodiments, polymers comprise one or more of mucin, hyaluronic acid, polyvinyl alcohol, sodium CMC, polyvinylpyrrolidone, hydroxypropyl methylcellulose, carbopol (e.g., Carbopol 934), and poloxamer (e.g., poloxamer 407). Mucoadhesive agents comprise but are not limited to alginic acid (sodium alginate) and sodium CMC. Viscosity agents comprise but are not limited to Guar gum and Carbopol 934.

Some excipients serve as pH modifiers and/or buffers, such as lactic acid and acetate buffer.

Suitable formulations show little to no flow on suitable vertical surfaces and maintain high bacterial viability (e.g., CFU count) both upon formulation and during (long-term) storage.

Desired formulation selection parameters include, for example, mucoadhesion (of reconstituted product, e.g., in the vaginal tract); viscosity (of reconstituted product), e.g., final viscosity for gel-based product needs to be syringeable at ambient temperature and preferably congealed at 37° C. (at body temperature, e.g., in the vaginal tract); total sugar content (of reconstituted product), e.g., ideally at or lower than physiological concentration (about 0.5-1.0 mg/mL); volume of reconstituted product, e.g., up to 3 mL; hydration rate/disintegration rate (e.g., of gel/matrix), e.g., sufficient physical integrity to provide desired release rate; pH, e.g., between about pH 3.4-3.9 (e.g., to promote inhibition of competitive vaginal bacteria); water activity/moisture content (e.g., of dried formulations), e.g., between 0.5-3% water (e.g., for longer term dried formulation stability); microbial diversity, e.g., relative abundance of Lactobacillus species, such as, e.g., L. crispatus, L. gasseri, L. jensenii, and L. iners; total dose/potency, e.g., preferably at least about 1×10⁵ CFU/VCC, at least about 10⁶ CFU/VCC, at least about 10⁷ CFU/VCC or at least about 10⁸ CFU/VCC per administration (per dose), shelf-life (not reconstituted) at various temperatures, and microbial limits, e.g., absence of microorganisms such as, Pseudomonas aeruginosa, Candida albicans, Staphylococcus aureus, Ph Eur criteria 5.1.4, 2.6.12 & 2.6.13).

Suitable testing methods include standard assays, such as, plate count (e.g., MRS agar), e.g., for life bacteria count, dose determination, shelf-life; rheometer, e.g., for mucoadhesion and viscosity, pH meter, Karl Fisher/water activity meter, Ph Eur testing, e.g., for microbial loads.

Substantially complete vaginal microbiota preparations can be lyophilized and

formulated into, e.g., gels and tablets as well as other dosage forms that can be filled with lyophilized products, such as, e.g., capsules. Substantially complete vaginal microbiota preparations can also be formulated into gels that can be frozen, as well as into liquid media (e.g., with glycerol) that can be frozen. Substantially complete vaginal microbiota preparations can also be formulated into (air-dried) films, that could be, e.g., shaped like disks. Losses in viability range from approximately 0.5 log to 1 log at the formulation step depending on excipient and dosage form.

In preferred embodiments, the substantially complete vaginal microbiota preparation of the invention is provided in a dosage form selected from a tablet, pre-formed gel, lyophilized gel, liquid formulation, frozen formulation, film-forming formulation or film.

Pre-Formed and Frozen Gels

The substantially complete vaginal microbiota preparation of the invention can be comprised in a pre-formed gel. In some embodiments, the pre-formed gel is provided in a vial, e.g., for drawing up into a syringe. In some embodiments, the pre-formed gel is provided in a suitable applicator, e.g., as shown in FIG. 13. The pre-formed gel may be stored frozen or refrigerated depending to maintain the stability of the substantially complete vaginal microbiota preparation. In some embodiments, the pre-formed gel comprises hyaluronic acid. In some embodiments, hyaluronic acid is comprised in a concentration of about 0.3-3%. In some embodiments, hyaluronic acid is comprised in a concentration of about 0.5-2%. In some embodiments, the pre-formed gel comprising hyaluronic acid may be frozen at −80° C. In some embodiments, the frozen pre-formed gel comprising hyaluronic acid is substantially stable for a time period of at least 3 months, 6 months, 9 months, 12 months, 18 months or longer.

Lyophilized Gels

The substantially complete vaginal microbiota preparation of the invention can be comprised in in a lyophilized gel. The lyophilized gel may is stable and may be stored for extended periods of time at 2-8° C. The lyophilized gel comprises the lyophilized substantially complete vaginal microbiota preparation and a gel forming excipient. The lyophilized gel may be supplied in a vial. The lyophilized gel can be reconstituted prior to administration (e.g., in a clinical or home setting) with a reconstitution agent. In some embodiments, the reconstitution agent comprises a gel, a gel forming agent, or a liquid. The liquid may comprise water, saline or another liquid suitable for reconstitution and subsequent administration to a subject.

In particular embodiments, the lyophilized gel comprising the substantially complete vaginal microbiota preparation of the invention further comprises sodium-carboxymethylcellulose (Na-CMC). In some embodiments, Na-CMC is comprised in a concentration of about 1-3%. In some embodiments, Na-CMC is comprised in a concentration of about 2%. In particular embodiments, the lyophilized gel comprises the substantially complete vaginal microbiota preparation of the invention and hyaluronic acid. In some embodiments, hyaluronic acid is comprised in a concentration of about 0.3-3%. In some embodiments, hyaluronic acid is comprised in a concentration of about 0.5-2%. In particular embodiments, the lyophilized gel comprises the substantially complete vaginal microbiota preparation of the invention and hyaluronic acid and sodium-carboxymethylcellulose (Na-CMC) at the above concentrations. The lyophilized gel comprising NA-CMP, hyaluronic acid, or a combination of both, is stable at 2-8° C. or at −20° C. for a time period of at least 3 months, 6 months, 9 months, 12 months, 18 months or longer. In some embodiments, the lyophilized gel comprising hyaluronic acid may be frozen at −80° C. In some embodiments, the frozen lyophilized gel comprising hyaluronic acid is stable for a time period of at least 3 months, 6 months, 9 months, 12 months, 18 months or longer. In some embodiments, the lyophilized gel and the reconstitution agent are provided as a kit.

Film Forming Formulations

The substantially complete vaginal microbiota preparation of the invention can be comprised in in a film-forming formulation. The substantially complete vaginal microbiota preparation of the invention can be formulated with polymeric excipients, wherein the polymeric excipients have bioadhesive properties and film-forming capacity. Exemplary polymeric excipients for film-forming formulations include polyvinyl alcohol (PVA), sodium lactate and lactic acid. In particular embodiments, the film-forming formulation comprises polyvinyl alcohol (PVA). In some embodiments, the PVA is comprised in a concentration of about 10-25%, 10-20%, or about 12-15%. In particular embodiments, the film-forming formulation comprises PVA in a concentration of about 12%. In some embodiment, the film-forming formulation comprises PVA, e.g. in a concentration of about 10-25%, 10-20%, 12-15% or 12%, and is air-dried. In some embodiments, the film-forming formulation is provided as a disc or wafer. In some embodiments, the film-forming formulation is provided as a mucoadhesive pessary or patch. The film-forming formulation is substantially stable for a time period of at least 3 months, 6 months, 9 months, 12 months, 18 months or longer at 2-8° C. In some embodiments, the film-forming formulation rapidly disperses or dissolves in contact with fluids, e.g. cervicovaginal solution, to intravaginally form a viscous and bioadhesive gel. In some embodiments, formation of a bioadhesive dispersion is retained in the vagina for prolonged periods of time.

Tablets

The substantially complete vaginal microbiota preparation of the invention can be comprised in in a tablet. The tablet may comprise agents with gel-forming properties, muco-adhesive properties, or a combination of both. The tablet may further comprise excipient, such as swelling agents, bulking agents, lactic acid, carbopol, HPMC, alginate, or sodium-carboxymethylcellulose (Na-CMC). In some embodiments, the bulking agent comprises microcrystalline cellulose, HPMC/PVP, maltodextran, or poloxamer 407. In particular embodiments, the tablet comprises the substantially complete vaginal microbiota preparation of the invention and Na-CMC. In particular embodiments, the tablet comprises the substantially complete vaginal microbiota preparation of the invention and polyvinylpyrrolidone (PVP). The tablet may further be substantially stable, e.g., substantially retain Lactobacilli viability. The tablet may be substantially stable at 2-8° C. or at room temperature (about 25° C.) for a time period of at least 3 months, 6 months, 9 months, 12 months, 18 months or longer. In particular embodiments, the tablet comprises the substantially complete vaginal microbiota preparation of the invention and sodium-carboxymethylcellulose (Na-CMC) and is stable at 2-8° C. or at room temperature (about 25° C.) for a time period of at least 3 months, 6 months, 9 months, 12 months, 18 months or longer. In particular embodiments, the tablet comprises the substantially complete vaginal microbiota preparation of the invention and polyvinylpyrrolidone (PVP) and is stable at 2-8° C. or at room temperature (about 25° C.) for a time period of at least 3 months, 6 months, 9 months, 12 months, 18 months or longer.

Liquid Formulations

The substantially complete vaginal microbiota preparation of the invention can be comprised in in a liquid formulation. In some embodiments, the liquid formulation may be provided as a frozen formulation. In some embodiments, the liquid formulation is provided with gelling agents in a kit. The liquid formulation may comprise a cyroprotectant, such as glycerol, wherein the glycerol concentration is optionally less than 25%, less than 20%, less than 15%, or less than 10%. In some embodiments, the liquid formulation may be frozen, e.g., at −20° C. or −80° C., and substantially retains Lactobacillus viability. In some embodiments, the liquid formulation further comprises lactic acid. In a preferred embodiment, the liquid formulation comprises lactic acid and a cryoprotectant, such as glycerol. In some embodiments, the frozen liquid formulation comprising the cryoprotectant, and optionally lactic acid, is substantially stable for a time period of at least 3 months, 6 months, 9 months, 12 months, 18 months or longer. In some embodiments, the liquid formulation comprising the substantially complete vaginal microbiota preparation is provided with a gelling agent as a kit. In some embodiments, a frozen liquid formulation comprising the substantially complete vaginal microbiota preparation is provided with a gelling agent as a kit. In some embodiments, a frozen liquid formulation which does not comprise the substantially complete vaginal microbiota preparation is provided with a lyophilized gel as a kit, wherein the lyophilized gel comprises the substantially complete vaginal microbiota preparation.

Kits

Provided herein are also kit comprising vaginal delivery systems, including one or more dosage forms or devices and other agents, as described herein, including, optionally, instructions for the user. In some embodiments, the dosage form or device and the composition are in separate sterile packages. In some embodiments, the dosage form and/or the composition comprises multiple doses. In some embodiments, the dosage form or device is ready-to-use. In another embodiment, the substantially complete vaginal microbiota preparation or composition thereof needs to be reconstituted. In such embodiments, the kit further comprises a medium for reconstitution. The kit may comprise single dosage forms or multi-dosage forms.

Methods of Producing Preparations and Compositions and Donor Considerations

Aspects of the invention include methods of producing a substantially complete vaginal microbiota preparation as described herein. These methods include selecting a suitable female donor to provide samples of vaginal fluids, preferably a cervicovaginal secretion, and processing the samples to provide compositions (such as pharmaceutical compositions) comprising the substantially complete vaginal microbiota preparation described herein. The donor female is generally healthy, does not exhibit dysbiosis of the vaginal microbiota and optionally is subjected to one or more additional health tests. The substantially complete vaginal microbiota preparation provided herein (i) comprises one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the preparation; and (ii) comprises less than 5% of Gardnerella spp., Atopobium spp., and Prevotella spp. For example, the methods of producing a substantially complete vaginal microbiota preparation includes a step of providing a microbiota sample (such as a cervicovaginal secretion) from a healthy female donor and a step of releasing from quarantine (e.g., based on meeting one or more predetermined (quality) parameters, such as, e.g., those obtained from performing one or more of steps 5(a) to 5(e) below) a processed sample as a (pharmaceutical) composition, e.g., for administration to a female recipient in need of a substantially complete vaginal microbiota preparation.

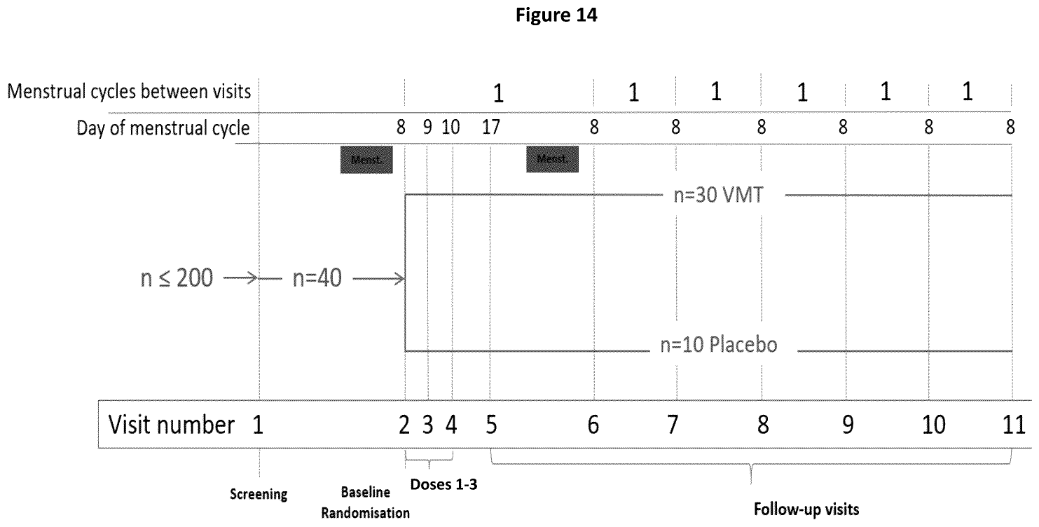

Generally, the step of providing and processing of a microbiota sample (such as a cervicovaginal secretion) from a healthy female donor can include one, two, three, four, five or more steps, and any combinations, e.g., any two, three, four, five or more steps, including: (1) adding a diluent (e.g., saline), buffer, or other excipient to the microbiota sample to create a diluted sample; (2) removing a portion of the diluted microbiota sample for testing (e.g., nucleic acid sequencing); (3) pre-cooling for either refrigeration or freezing of the remainder of the microbiota sample; (4) storing the refrigerated or frozen microbiota sample under quarantine, and/or (5) holding the refrigerated or frozen microbiota sample under quarantine until completion of any combination of (a), (b), (c), (d), and/or (e): (a) testing the donor to exclude the presence of transmissible pathogens (e.g., blood, vaginal swab, and/or urine sample testing), (b) confirming the composition and viability of the donor sample microbiota (e.g., lactobacilli), (c) further confirming the health of the female donor by a plurality of post-screening tests occurring within a time period of 30-90 days post-donation (or alternatively: 10-120 days, or 30-60 days), (d) testing for presence of sperm cells, and/or (e) testing pH of the sample (e.g., Figure. 14). Any of steps (1) to (5) and 5(a) to 5(e) are optional and can be carried out sequentially or in parallel in any particular order that is desired.

In one embodiment, the method of producing a substantially complete vaginal microbiota preparation comprises
A. providing a microbiota sample from a donor female genitourinary tract; wherein step A comprises one, two, or three of steps (1), (2), (3) or any combination thereof, and both steps (4) and (5):
   (1) adding a diluent to the microbiota sample to create a diluted sample,
   (2) removing a portion of the diluted microbiota sample for testing (e.g., nucleic acid sequencing),
   (3) pre-cooling for either refrigeration or freezing of the remainder of the microbiota sample,
   (4) storing the refrigerated or frozen microbiota sample under quarantine,
   (5) holding the refrigerated or frozen microbiota sample under quarantine until any completion of any combination of (a) testing the donor to exclude the substantial presence of one or more transmissible pathogens (e.g., blood, and/or cervicovaginal secretions, and/or urine sample testing), (b) confirming the composition and viability of the microbiota, or (c) further confirming the health of the female donor by a plurality of post-screening tests occurring within a time period of 30-90 days post-donation; and
B. releasing the refrigerated or frozen microbiota sample from quarantine to define the substantially complete vaginal microbiota preparation.

Step (1) may include, e.g., adding an acidifying agent (e.g., to adjust the pH of the sample); adjusting the viscosity of the sample (e.g., to aid administration as described herein); adjusting the isotonicity/osmolarity; and/or adding one or more other active agents, such as described herein, including spermicides, antimicrobial agents, hormonal agents, anti-inflammatory agents, and optionally prebiotics. In one embodiment, the acidifying agent is lactic acid.

For step (2), the microbiota sample (e.g., a sample of vaginal fluid/vaginal secretion) is preferably at least 75 mg or 100 mg, more preferably at least 150 mg and a portion is removed for nucleic acid sequencing. The microbiota sample may be 75 mg, 100 mg, 150 mg, 200 mg, 250 mg, or 300 mg. In a further embodiment, the microbiota sample (e.g., a sample of vaginal fluid/vaginal secretion) is at least 200 mg, 300 mg, 400 mg, 500 mg, 500 mg, 700 mg or more. In one embodiment, the microbiota sample (e.g., a sample of vaginal fluid/vaginal secretion) is at least 500 mg.

Sequencing is performed to assess the microbial community of the donor microbiota sample and to select suitable donor females. Preferably, the presence of one, two, three, four or five different bacterial species from the genus Lactobacillus is detected in the donor microbiota sample by nucleic acid sequencing. Most preferably, one (dominant) bacterial species from the genus Lactobacillus is detected in the donor microbiota sample by nucleic acid sequencing. In some embodiments, nucleic acid sequencing determines that the donor microbiota sample comprises 80%, about 85%, about 90%, about 91%, about 92%, about 93%, about 94%, about 95%, about 96%, about 97%, about 98%, about 99%, about 99.5%, about 99.9%, 80-99.9%, 75%-95%, 85%-95%, 85%-99.9%, or 90%-99.9% lactobacilli of one species of the total of all detectable species in the preparation. In some embodiments, nucleic acid sequencing determines that the donor microbiota sample comprises 80%, about 85%, about 90%, about 91%, about 92%, about 93%, about 94%, about 95%, about 96%, about 97%, about 98%, about 99.9%, about 99.5%, about 99.9%, 80-99%, 75%-95%, 85%-95%, 85%-99.9%, or 90%-99.9% lactobacilli of more than one species (e.g., two, three, four, or five species) of the total of all detectable species in the preparation. Preferably, nucleic acid sequencing determines that the donor microbiota sample comprises species of Lactobacillus selected from: a) Lactobacillus crispatus; (b) Lactobacillus iners; (c) Lactobacillus jensenii; (d) Lactobacillus gasseri, or any combination thereof (including combinations of two, three, or all four species).

Optionally, nucleic acid sequencing is performed to identify any pathogens or pathobionts in the donor sample, e.g., to determine that a donor sample is substantially free of pathogens and pathobionts. For example, nucleic acid sequencing is preformed to detect the presence of one or more of Gardnerella spp., Atopobium spp., and/or Prevotella spp. In some embodiments, nucleic acid sequencing is preformed to detect the presence of one or more of Gardnerella spp., Atopobium spp., Prevotella spp, and/or Fannyhessa vaginae. In some embodiments, nucleic acid sequencing is performed to detect the presence of one or more of Gardnerella vaginalis, Bacteroides, Mobiluncus spp., Sneathia spp., and Mycoplasma hominis. In some embodiments, nucleic acid sequencing is preformed to detect the presence of one or more of Escherichia, Enterococcus, Pseudomonas, Proteus, Klebsiella, Streptococcus, Staphylococcus, Gardnerella, Ureaplasma, Bacteroides, Peptococcus, Neisseria, Serratia, Corynebacterium, Clostridium, and Candida. The presence of any one or more of these species suggests the displacement of healthy vaginal lactic acid producing bacteria by unwanted species, signaling the presence of vaginal dysbiosis. Donor samples containing more than about 1%, 3%, 5%, 8%, 10% or more than about 15% of species belonging to one or more unwanted species are not used for further processing to generate the substantially complete vaginal microbiota preparations described herein. In one embodiment, donor samples containing more than about 5% of species belonging to one or more unwanted species (e.g., are Gardnerella spp., Atopobium spp., and/or Prevotella spp.) are not used for further processing to generate the substantially complete vaginal microbiota preparations described herein.

Optionally, nucleic acid sequencing is performed to identify the presence of any antimicrobial resistance (AMR) genes in the donor sample, e.g., to determine that a donor sample is substantially free of antimicrobial resistance (AMR) genes. Antimicrobial resistance (AMR) genes include genes that confer resistance to one or more antibiotics, including, e.g., aminoglycosides, beta-lactams, tetracyclines, and sulfonamides (e.g., as described and cataloged in the NCBI National Database of Antibiotic Resistant Organisms (NDARO)). It will be appreciated that due to extensive use of antibiotics the cut-off is not zero. Some reasonable allowance for the presence of AMR genes is made. The precise cut-off can be determined by one of ordinary skill, based on, e.g., the nature of the AMR genes (e.g., degree of health concern) and public health recommendations.

Alternatively, or in addition, tests are performed to determine that a donor sample is substantially free one or more of gram-negative toxins (e.g., endotoxin or lipopolysaccharide (LPS) and other secreted exotoxins and enterotoxins), pathogenicity factors/bacterial virulence factors, and/or colonization factors (e.g., motility, adherence, invasiveness, etc.).

Optionally, tests are performed (e.g., by microscopy) to identify the presence of any human sperm (spermatozoa) in the donor sample, e.g., to determine that a donor sample is substantially free of human sperm (spermatozoa).

Step (3) may include pre-cooling for either subsequent refrigeration (e.g., at 4° C.) or freezing (e.g., −18° C. or −80° C.), depending on the desired time period for storage, quarantine and use for administration. This step may also include freeze-drying (lyophilizing), e.g., for easy storage, packaging, formulation and transport. Further, this step may optionally include viability testing, e.g., upon refrigeration, freezing, or freeze-drying, e.g., viability of the lactobacilli comprised in the substantially complete vaginal microbiota preparations.

Steps (4) and (5) include storing the refrigerated, frozen or freeze-dried microbiota sample under quarantine, and holding the stored microbiota sample under quarantine until completion of a number of tests conducted on the donor microbiota sample and/or the sample donor. The quarantine is lifted, and the sample released for use as a substantially complete vaginal microbiota preparation or composition (e.g., pharmaceutical composition), e.g., for administration to a recipient female, upon the sample and/or the donor passing one or more predetermined tests (sample quality and/or female donor health tests). Those include one or more of: (a) testing the donor to exclude the presence of transmissible pathogens (e.g., blood, vaginal swab, and/or urine sample testing); (b) confirming the composition and viability of the donor sample microbiota (e.g., lactobacilli), and/or (c) further confirming the health of the female donor by a plurality of post-screening tests occurring within a time period of 30-90 days post-donation (or alternatively: 10-120 days, or 30-60 days).

Step 5 testing to exclude the presence of transmissible (and potentially infectious) pathogens (e.g., blood, vaginal swab, and/or urine sample testing) may include determining that the female donor is substantially free of any one or more (two or more, three or more, or four or more) of: (i) bacteria involved in bacterial vaginosis (e.g., Gardnerella and Mobiluncus), (ii) yeast (e.g., Candida, Cryptococcus, and Saccharomyces species), (iii) sexually transmitted pathogens (including Neisseria gonorrhea, Chlamydia trachomatis, and Trichomonas vaginalis), (iv) bacteria involved in urinary tract infections (e.g., E. coli, Staphylococcus, Chlamydia, and Mycoplasma), and (v) viruses (e.g., HIV, human papilloma virus (HPV), hepatitis B virus, hepatitis C virus, HSV-2).

Step 5 testing may include determining that the female donor is substantially free of any one or more sexually transmitted infections or diseases (STI, STD), including chlamydia, chancroid, crabs (pubic lice), genital herpes, genital warts, Hepatitis B, human immunodeficiency virus/acquired immunodeficiency syndrome, human papilloma virus, trichomoniasis, molluscum contagiosum, pelvic inflammatory disease, syphilis, gonorrhea, and yeast infections.

Step 5 testing may include determining that the female donor does not exhibit a dysbiosis in the vaginal tract, e.g., by one or more established tests for bacterial vaginosis (BV) and bacterial infections. Such tests include Amsel Criteria, Nugent Gram-stain scoring system, and Hay-Ison Criteria. Alternatively, other methods may be used to determine the absence of dysbiosis, e.g., using the BV Blue test or Affirm Microbial Identification Test.

Amsel Criteria include the presence of three of the following four symptoms: (a) thin homogeneous malodorous discharge; (b) vaginal pH fluid>4.5; (c) an amine odor from vaginal fluid when 10% KOH is added; and (d) the presence of “clue” cells (vaginal epithelial cells with adherent bacteria that obscure cell margins) (Amsel et al., Am. J. Med. 74:14-22 (1983)).

The Nugent Gram-stain scoring system involves assessment of a normally prepared Gram stain for relative abundance of three morphotypes of bacteria, and then calculating the so-called Nugent score based on the amounts of large Gram-positive rods (lactobacilli morphotype; decrease in lactobacilli is scored as 0 to 4), small Gram-negative and variable rods (Bacteroides and Gardnerella morphotype; scored as 0 to 4), and curved gram-variable rods (Mobiluncus spp. morphotype; scored as 0 to 2). The Nugent score can range from 0 to 10, with scores of 0-3 deemed normal (non-BV), 4-6 intermediate, and 7-10 positive for BV.

Hay-Ison Criteria (alternatively Ison-Hay scoring system) suggests five grades of flora: a) Grade 0, epithelial cells with no bacteria; b) Grade I, normal vaginal flora (lactobacillus morphotypes alone); c) Grade II, reduced numbers of lactobacillus morphotypes with a mixed bacterial flora; d) Grade III, mixed bacterial flora only, few or absent lactobacillus morphotypes; e) Grade IV, Gram positive cocci only.

Grades 0, I, and IV are found in women without BV. Grade II is intermediate and not found in women with BV as defined by Amsel criteria. Grade III is consistent with BV as diagnosed by Amsel criteria. Grade III flora are indicative of BV (C. A. Ison and P. E. Hay, Sex Transm. Infect. 2002 December; 78(6):413-5).

The BV BLUE test (Gryphus diagnostics) detects sialidase activity, an enzyme produced by BV-associated bacteria such as Gardnerella vaginalis, Bacteroides spp., Prevotella spp., and Mobiluncus spp. A vaginal fluid sample is placed in the test vessel which contains a chromogenic substrate for sialidase. After incubation, a developer solution is added, and if the sample contained a high level of sialidase, a blue or green color is seen. Samples containing no sialidase, or low levels of this enzyme, will generate a yellow color in the reaction.

The AFFIRM Microbial Identification Test (Beckton Dickinson) is a DNA probe-based diagnostic test for the differential detection and identification of the three types of vaginitis-associated organisms: Candida spp., G. vaginalis and T. vaginalis.

In some embodiments, donor females are selected from generally healthy, pre-menopausal women, of ages 18 years and older with regular, predictable menstrual cycles. In some embodiments, donor females are selected from generally healthy, post-menopausal women. In some embodiments, donor females are selected from both pre- and post-menopausal women. Donors can take oral contraceptives, hormonal contraceptives, hormonal intrauterine devices or no contraceptives. Donors are substantially free of vaginal symptoms, such as odor, discharge, or itching. Optionally, donors do not use or perform one or more of (or all of a-e) during the sample donation period, e.g., from initial donor screening to the final donation: a) use vaginal feminine products that are inserted, e.g. tampons, menstrual cups, sex toys, though sanitary napkins are acceptable; b) use other vaginal products, such as, e.g., cleansing products, spermicides, lubricants, hygiene powders and sprays); c) have vaginal and anal intercourse; d) take baths, go swimming, sit in a hot tub, and/or e) wear thong underwear.

Donors may be excluded if they exhibit one or more of the following (e.g., test above a set of predetermined thresholds, e.g., concerning viral, fungal, and bacterial pathogen and/or pathobiont load, for which individual maximal thresholds may be set, e.g., above zero, such as, e.g., being substantially free thereof): (a) a health history of one or more of: bacterial vaginosis, recurrent yeast infection, trichomoniasis, syphilis, human papilloma virus (HPV) including genital warts, high grade pap smear, herpes, pelvic inflammatory disease, recurrent urinary tract infection, and mycoplasma, or any combination thereof; (b) testing positive for one or more of: HIV, Hepatitis A/B/C, syphilis, Human T-lymphotrophic Virus (HTLV)-I/II, WNV, Epstein-Barr Virus (EBV), rubella, Toxoplasma gondii, Herpes Simplex Virus (HSV)-1/2, chlamydia, gonorrhea, Mycoplasma genitalium, Trichomonas vaginalis, HPV, and other yeast or bacteria that are considered pathogenic/abnormal and/or show antibiotic resistance, or any combination thereof, (c) vaginal fluid/cervicovaginal secretions not dominated by one of the common vaginal lactobacillus species, e.g., as determined by qPCR; and/or (d) history of gonorrhea or chlamydia (e.g., within 12 months prior to screening), or any combination of (a), (b), (c) and (d).

Further, donors may be excluded if they exhibit one or more of the following: hysterectomy, intra-uterine device insertion or removal, cervical cryotherapy, or cervical laser treatment (e.g., within 2 months prior to screening), any condition requiring regular periodic use of systemic antibiotics, use of long-acting hormonal treatments, social, medical, or psychiatric condition, including history of drug or alcohol abuse, menopause (e.g., defined as more than 12 consecutive months of amenorrhea without another known cause), irregular menstrual cycles, use of other medication, or any combination thereof. Preferably, donors are not currently pregnant or breastfeeding.

If desired, donors exhibiting one or more of cytomegalovirus (CMV), Rubella, and Varicella Zoster Virus (VZV) IgG, or any combination thereof, will only be matched with CMV positive and/or Rubella and/or VZV positive recipients, or can be excluded.

Methods of Administration

Aspects of the invention include methods for vaginal administration to a human female subject of a composition comprising a substantially complete vaginal microbiota preparation described herein. The methods may include using a device for administration, e.g., these methods would normally be carried out by a healthcare provider (e.g., in a clinic). For example, the composition comprising a substantially complete vaginal microbiota preparation is provided frozen, e.g., in a cryo-vial, then thawed and pre-heated to 37° C. and then dispensed into a syringe/applicator by a healthcare provider and then administered to a recipient. Alternatively, the methods include using an alternative dosage form described herein, such as, e.g., a suppository, tablet, capsule, film, cream, etc. The methods can, if desired, be carried out by the recipient herself, e.g., by self-administration (e.g., at home). The methods may also include other healthcare related activities, such as diagnosing a health issue and providing standard of care in addition to providing a substantially complete vaginal microbiota preparation or composition described herein. The activities can include the one or more combination therapies provided herein. For example, the methods for administration described herein may further comprise administering antimicrobial agents, antifungal agents, antibacterial agents, antiviral agents, antibiotics, antiparasitic agents (e.g., with activities against Trichomonas vaginalis), anti-inflammatory agents, and the like.

In embodiments, in which administration is carried out using a device, the device typically includes an open end (e.g., a tip) for insertion into the vaginal cavity, and a dispensing end (e.g., a plunger or piston) to expel the composition through the open end. The administration steps include: a) introducing the open end into a vaginal cavity, b) expelling the composition into the vaginal cavity, c) removing the device from the vaginal cavity (after administering the desired dose). Administration is preferably carried out with the recipient being in a lithotomy position, e.g., with the female recipient in a lithotomy position. In some embodiments, the recipient is to remain in a lithotomy position for at least 5 minutes, at least 10 minutes, at least 20 minutes, at least 30 minutes before returning to an upright position to allow the substantially complete vaginal microbiota preparation or composition sufficient residence time in the vaginal cavity, e.g., sufficient contact time with the mucosal or endometrial surfaces of the vagina. In some embodiments, the administration is carried out targeting areas high in the vaginal cavity, e.g., near the vaginal fornices.

If desired, the menstrual cycle of the recipient female is taken into account when determining the timing of administration. For example, the procedure may be avoided during menstrual discharge. In some embodiments, a time other than during menstrual discharge is preferred for carrying out the administration, including, e.g., during a time window that includes prior to ovulation and prior to menstrual discharge.

In some embodiments, the precise steps, timing, and length of the procedure varies between recipients (and is, e.g., determined by a healthcare provider) in order to provide optimal conditions for the bacteria (e.g., lactobacilli) comprised in the substantially complete vaginal microbiota preparation to colonize and become established (engrafted) in the vagina of the recipient female.

Methods of Treatment and Recipient considerations

Suitable female recipients include females exhibiting a dysbiosis of the vaginal microbiota and those that are in need of treatment for dysbiosis, such as dysbiosis associated with an infection (e.g., with a pathogen), and/or (chronic) inflammation.

Aspects of the invention relate to methods for restoring a healthy human vaginal microbiota balance in the female genitourinary tract. The methods include administering to a female subject in need of restoration of vaginal microbiota balance an effective amount of a composition comprising a substantially complete vaginal microbiota preparation described herein for the purpose of providing a community of microbial species that are capable of colonizing or inhabiting the human vagina or vaginal epithelium and that provide a microbial niche that discourages the growth of pathogenic microbes and is not pro-inflammatory (e.g., is anti-inflammatory). The methods include administering to a female subject in need of vaginal microbiota balance an effective amount of a composition comprising a substantially complete vaginal microbiota preparation described herein for the purpose of maintaining healthy vaginal microbiota or a healthy vaginal microbiota balance.

In one aspect, the invention provides a pharmaceutical composition for use in treating inflammation in the female genitourinary tract of a human subject, wherein the female subject exhibits a dysbiotic microbiota in the genitourinary tract, said method comprising administering to the subject an effective amount of the pharmaceutical composition, wherein the pharmaceutical composition comprises a substantially complete vaginal microbiota preparation,
wherein (i) the preparation
   (i) comprises one, two, three or four bacterial species from the genus Lactobacillus, selected from Lactobacillus crispatus, Lactobacillus iners, Lactobacillus jensenii, Lactobacillus gasseri, which comprise >80-99.9% of all detectable bacterial species of the preparation; and
   (ii) comprises <5% of Gardnerella spp., Atopobium spp., and Prevotella spp.; wherein the pharmaceutical composition comprises a pharmaceutically acceptable carrier or diluent.

Inflammation and disorders, conditions or diseases associated with inflammation are used interchangeably and comprise acute and chronic inflammation. Dysbiotic vaginal microbiota may be associated with inflammation.

The subject may be an asymptomatic subject, wherein an asymptomatic subject is characterized by having no Amsel's criteria, Nugent score or other measures of symptoms associated with bacterial vaginitis (BV). In one aspect, the recipient is a asymptomatic, dysbiotic woman.

Vaginal Wash

In order to reduce the quantity of the pathogens in the vaginal cavity of the dysbiotic recipient, in some embodiments, prior to administering the pharmaceutical composition the vaginal cavity is rinsed, e.g., a vaginal wash is performed.

The vaginal wash may be performed with an absorbent material pre-soaked in the wash solution. In one embodiment, the absorbent material is gauze. In some embodiments, a tampon-shaped gauze may be used. The wash is typically performed by a medical professional, such as a gynecologist, wherein the recipient is in the lithotomy position and the pre-soaked absorbent material is used with pliers to wash the surface of the vaginal epithelium. After performing the vaginal wash, excess wash solution may be absorbed by a dry absorbent material, e.g., a dry tampon-shaped gauze.

The vaginal wash can be performed with any solution that is suitable for reducing the quantity of pathogens in the vaginal cavity, including but not limited to saline, antiseptic, or lactic acid. In some embodiments, the vaginal wash comprises a saline vaginal wash, lactic acid wash, or wash with an antiseptic solution. Suitable antiseptic solutions include chlorohexidine and povidone-iodine. In a particular embodiment, the vaginal wash is performed with saline. In another particular embodiment, the vaginal wash is performed with lactic acid, optionally wherein the lactic acid has a pH of about 3.5-4.5. In a particular embodiment, the vaginal wash is performed with lactic acid having a pH of about 3.8-4.3 or 3.8 to about 4.0. In a further particular embodiment, the vaginal wash is performed with an antiseptic solution, optionally wherein the antiseptic solution is chlorohexidine or povidone-iodine. In a particular embodiment, the vaginal wash is performed with povidone-iodine, wherein the solution comprises about 10% povidone-iodine. In a particular embodiment, the vaginal wash is performed with chlorhexidine, wherein the solution comprises about 0.5% chlorhexidine.

Homeostasis of Vaginal Microbiota

A healthy human vaginal microbiota balance includes establishment (including, e.g., engraftment) of a select variety of microbial species (including one or more Lactobacillus species) in which the relative numbers of each species or the sum of vaginal Lactobacilli (e.g. Lactobacillus, selected from Lactobacillus crispatus, Lactobacillus iners, Lactobacillus jensenii, Lactobacillus gasseri) are in homeostasis, as measured post administration of a composition comprising the substantially complete vaginal microbiota preparations described herein. Homeostasis, generally, is a state in which the relative abundance of each species in a population does not substantially vary over a given period of time, e.g., at least for 1 day, 3 days, 5 days, 7 days, 10 days, 14 days, 21 days, 30 days, 60 days, 90 days, 6 months, or at least 12 months, or, e.g., over several (1, 2, 3, 4, 5, 6) menstrual cycles in a reproductive age woman. Homeostasis may be measured, e.g., by metagenomic sequencing, such as MLST and MLVA, 16S sequencing and/or qPCR. If desired, CFU counts may be taken, e.g., to measure persistence of colony forming units of healthy bacteria).

A healthy human vaginal microbiota balance at a homeostatic state (preferably rich in lactobacilli, e.g., at least 50%, 60%, 70% or at least 80% of lactobacilli, e.g., selected from one or more of: L. crispatus, L. iners, L. gasseri and L. jensenii) confers, e.g., resistance to perturbations caused by vaginal pathogens (non-pathogenic) and/or provide an anti-inflammatory environment and is stable for a period of time. However, a healthy vaginal microbiota does not require complete absence of all pathogens. Many healthy women have low levels of yeast and/or pathobionts present in their microbiota.

In some embodiments, the methods for restoring a healthy human vaginal microbiota balance in the female genitourinary tract include providing a non-proinflammatory or anti-inflammatory environment in the genitourinary tract. In some embodiments, the compositions comprising a substantially complete vaginal microbiota preparation described herein when administered (e.g., for treatment) provide anti-inflammatory activity to the genitourinary tract. In some embodiments, the compositions comprising a substantially complete vaginal microbiota preparation described herein when administered (e.g., for treatment) do not provoke a local or systemic inflammatory or immune response in the subject or recipient. In some embodiments, the compositions comprising a substantially complete vaginal microbiota preparation described herein when administered (e.g., for treatment) promote a local or systemic anti-inflammatory or immune response in the subject or recipient. In some embodiments, the compositions comprising a substantially complete vaginal microbiota preparation described herein when administered (e.g., for treatment) provide maintenance of the balance of anti-inflammatory and proinflammatory mediators in vaginal epithelial cells of the genitourinary tract.

Inflammatory Markers

Proinflammatory cytokines that can be modulated by the methods described herein include, e.g., IL-1β, IFN-γ, IL-2, IL-4, IL-6, IL-8, IL-10, IL-12p70, IL-13, IL-17, IL-23, and TNF-α. In one embodiment, the administration of the substantially complete vaginal microbiota preparation effects reducing or stabilizing the levels of inflammatory markers, wherein the markers comprise one or more of IL-1a, IFN-γ, IL-18, IL-12, and MMP-10. In some embodiments, anti-inflammatory markers are increased after administration of the preparation of the invention.

Anti-inflammatory and proinflammatory mediators upon administration and engraftment of the lactobacilli comprised in the substantially complete vaginal microbiota preparations described herein can be measured (e.g., collecting a vaginal sample (e.g., vaginal fluid/secretion) or a systemic sample, (e.g., blood) using suitable commercially available biomarker panels.

Provided herein are methods of treating (chronic) inflammation of the female genitourinary tract. The methods include administering to a female subject in need of treatment an effective amount of a composition comprising a substantially complete vaginal microbiota preparation described herein. Subjects treated by the methods described here include females exhibiting an inflammation (in some instances chronic inflammation) that may result in a number of adverse health outcomes, such as urinary tract infections (UTIs).

Dysbiosis

Provided herein are methods of treating dysbiosis of the female genitourinary tract. The methods include administering to a female subject in need of treatment an effective amount of a composition comprising a substantially complete vaginal microbiota preparation described herein. Subjects treated by the methods described here include females exhibiting an imbalance in the vaginal microbiota that may result in overgrowth of pathogenic microorganisms and pathobionts, resulting in dysbiosis, inflammation, and/or infections. The methods include the treatment of vaginal infections (vaginitis) such as bacterial vaginosis, vaginal candidiasis and trichomoniasis and combinations thereof with composition comprising a substantially complete vaginal microbiota preparation described herein.

Vaginal dysbiosis is a prevalent condition that occurs in about 30-40% of female adult subjects. In some embodiments, the dysbiotic female subject does not exhibit any symptoms (asymptomatic). In other embodiments, the dysbiotic female subject suffers from vaginal symptoms characterized by Amsel's criteria, Nugent score criteria or Hay-Ison criteria.

Bacterial vaginosis (BV) is the most common vaginal infection and is associated with complications during pregnancy as well as an increased risk for sexually transmitted diseases. It is caused by an imbalance of naturally occurring bacterial flora, wherein the lactobacilli are overgrown by a mixed flora of anaerobic bacteria. Amongst other diagnostic criteria, a pH greater than 4.5 is thought to be suggestive of bacterial vaginosis. BV may be diagnosed using Amsel criteria, Nugent score, Hay-Ison criteria, or another suitable method of diagnosis. Current treatment options rely on oral or vaginal administration of classical antibiotics such as metronidazole and clindamycin, however there is a high rate of recurrence of over 50% within 3 months after the first exposure. Alternative treatment options include acidification of the vagina with naturally occurring acids such as lactic acid or acetate.

In one embodiment, a female subject exhibiting one or more diagnostic signs of BV is treated with an effective amount of a composition comprising a substantially complete vaginal microbiota preparation described herein. In one embodiment, treatment comprising administration of an effective amount of a composition comprising a substantially complete vaginal microbiota preparation further comprises a combination therapy (as described herein), including providing standard of care treatment for BV, such as, e.g., administering antibiotics such as metronidazole and clindamycin and/or acidifying the vagina with an acidifying agent, such as, e.g., lactic acid or acetate, either prior to, concomitant with or after administration of the composition comprising a substantially complete vaginal microbiota preparation to the female subject.

Vaginal candidiasis is a fungal infection of any of the Candida species (yeasts), of which Candida albicans is the most common. Most Candida infections are treatable and result in only minimal complications such as redness or itching. However, complications may also be severe or even fatal if left untreated in certain populations, such as immuno-compromised patients. External use of detergents or internal disturbances (hormonal or physiological) can disturb the normal vaginal flora and result in an overgrowth of Candida cells causing infection. Pregnancy and the use of oral contraceptives have also been reported as risk factors. In clinical settings, candidiasis is commonly treated with antimycotics such as clotrimazole, nystatin, fluconazole and ketoconazole. However, certain yeasts such as, e.g., C. albicans can develop resistance to said antimycotic drugs.

In one embodiment, a female subject exhibiting one or more diagnostic signs of candidiasis is treated with an effective amount of a composition comprising a substantially complete vaginal microbiota preparation described herein. In one embodiment, treatment comprising administration of an effective amount of a composition comprising a substantially complete vaginal microbiota preparation further comprises a combination therapy (as described herein), including providing standard of care treatment for candidiasis, such as, e.g., administering antimycotics such as, e.g., clotrimazole, nystatin, fluconazole and/or ketoconazole, either prior to, concomitant with or after administration of the composition comprising a substantially complete vaginal microbiota preparation to the female subject.

Trichomoniasis is a sexually transmitted disease of the urogenital tract and is caused by the parasite Trichomonas vaginalis. Symptoms include inflammation of the cervix, urethra and vagina, which produce an itching and burning sensation. Treatment options include the use of antibiotic/anti-protozoal (e.g., metronidazole) or anti-parasitic (e.g., tinidazole) drugs. However, as with most anti-microbial drugs, resistance may occur.

In one embodiment, a female subject exhibiting one or more diagnostic signs of Trichomoniasis is treated with an effective amount of a composition comprising a substantially complete vaginal microbiota preparation described herein. In one embodiment, treatment comprising administration of an effective amount of a composition comprising a substantially complete vaginal microbiota preparation further comprises a combination therapy (as described herein), including providing standard of care treatment for Trichomoniasis, such as, e.g., administering antimycotics such as, e.g., antibiotic/anti-protozoal (e.g., metronidazole) or anti-parasitic (e.g., tinidazole) agents, either prior to, concomitant with or after administration of the composition comprising a substantially complete vaginal microbiota preparation to the female subject.

### Definitions

The definitions hereinafter apply to all aspects disclosed herein. For example, the term “substantially complete vaginal microbiota preparation” shall have the same meaning when used in the context of the first aspect which pertains to vaginal delivery system, or when used in the context of the second aspect which pertains to pharmaceutical composition, or when used in the context of any other aspect disclosed herein.

General

Unless otherwise explained, all technical and scientific terms used herein have the same meaning as commonly understood by one of ordinary skill in the art to which this disclosure belongs. It is further to be understood that methods and materials similar or equivalent to those described herein can be used in the practice or testing of this disclosure. The materials, methods, and examples are illustrative only and not intended to be limiting. Other features of the disclosure are apparent from the following detailed description and the claims.

The singular terms “a,” “an,” and “the” include plural references unless context clearly indicates otherwise. Similarly, the word “or” is intended to include “and” unless the context clearly indicates otherwise. The term “comprises” means “includes.” The abbreviation, “e.g.” is derived from the Latin exempli gratia, and is used herein to indicate a non-limiting example. Thus, the abbreviation “e.g.” is synonymous with the term “for example.” The term “optional” or “optionally” means that the subsequent described event, circumstance or substituent may or may not occur, and that the description includes instances where the event or circumstance occurs and instances where it does not.

Unless otherwise indicated, all numbers expressing quantities of components, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art.

In a preferred embodiment, the term “about” shall allow a deviation of ±10%, and even more preferably of ±5% from an indicated numerical value.

The term “comprising” shall be understood to simultaneously also disclose the term “consisting” as a preferred option. For example, if a composition is said to comprise three components, this also discloses a composition consisting of these three components as preferred embodiment.

If the term “comprising” is used when referring to (a) pharmaceutically active compound(s), bacterium(a), and the like, this shall be understood to simultaneously also disclose that the pharmaceutically active compound(s), bacterium(a), and the like is/are preferably the sole pharmaceutically active compound(s), bacterium(a), and the like. For example, if a donor sample is said to comprise Lactobacillus crispatus and Lactobacillus iners, this simultaneously and preferably discloses that a donor sample contains Lactobacillus crispatus and Lactobacillus iners as the sole bacteria. If a donor sample is said to comprise Lactobacillus crispatus, Lactobacillus iners, and an excipient, this simultaneously and preferably thus discloses that a donor sample contains Lactobacillus crispatus and Lactobacillus iners as the sole bacteria and in addition comprises an excipient. It further discloses, that the donor sample consists of Lactobacillus crispatus, Lactobacillus iners, and an excipient.

All patents, patent applications, and publications identified are expressly incorporated herein by reference for the purpose of describing and disclosing, for example, the methodologies described in such publications that might be used in connection with the present invention.

“Genitourinary tract” or “urogenital tract” as used herein as used herein includes the uterus, fallopian tubes, ovaries, vagina, cervix, vulva, and urinary tract. In some instances, used herein are teachings and exemplifications specifically calling out the “vaginal tract”, “vaginal cavity” or “vagina”. One of ordinary skill will appreciate that these exemplifications and teachings are illustrative only and non-limiting and, thus also apply, where appropriate, to other anatomical sites of the genitourinary tract or urogenital tract, not just to the vagina.

“Dysbiosis” as used herein means a microbial imbalance where normally predominant species are diminished in abundance and less predominant species become more abundant and/or predominant. Vaginal dysbiosis is a microbial imbalance in the vagina, an aberration of the healthy state. Dysbiosis is generally associated with one or more of: (a) qualitative and quantitative changes in the content or amount of the microbiota itself, (b) changes in their metabolic activities; and/or (c) changes in their local distribution. A dysbiotic human vaginal microbiota balance refers to a population of vaginal microbes that promotes inflammation of a tissue of the vagina and/or that contributes to or establishes an environment that permits or promotes the colonization or growth of one or more pathogenic microbes. Dysbiosis also refers to a perturbation of the vaginal homeostasis. In some embodiments, a dysbiotic vaginal microbiota will generally result in increased pH relative to a healthy microbiota, e.g., a pH above 4.5, e.g., a pH of 5.0, 5.5, 6.0, 6.5, 7.0 or higher. In some embodiments, vaginal dysbiosis is characterized by a reduction of Lactobacillus spp., and an increased diversity of vaginal anaerobic bacteria. Vaginal dysbiosis is associated with upper genital tract infections or pelvic inflammatory disease (PDI), and increased risk of sexual transmitted diseases. Dysbiosis may be characterized by the relative amount of selected pathogens, such as >20% selected pathogens, and the relative low abundance of vaginal lactobacilli, e.g. <10% vaginal lactobacilli (L. crispatus, L. iners, L. jensenii, L. gasseri).

Dysbiotic subjects as used herein comprise subjects having vaginal symptoms (symptomatic subjects) and subjects not having any vaginal symptoms (asymptomatic subjects), wherein symptoms are characterized by Amsel's criteria, Nugent score and/or Hay/Ison score.

In contrast, the term “normal” or “healthy” “vaginal flora” or “vaginal niche” “microbiota” or “state” or similar terms connote that a woman has no vaginal complaints and does not exhibit a vaginal pathology (e.g., no sign or symptom corresponding to or resulting from a pathology of the vagina), and the condition of the vagina is such of a relatively low susceptibility to sexually transmitted diseases and pathogens, and generally of low pH, e.g., less than or equal to pH 4.5, e.g., between 3.2 and 4.5; and generally dominated by lactic acid producing bacteria (e.g., Lactobacillus spp.). Normal vaginal microbiota or normal flora are a community of microorganisms that localize to the vagina in a normal, healthy, that is, a non-pathological, non-pathogenic and/or non-dysbiotic, state.

“Cervicovaginal secretions” (“CVS”) or “vaginal fluid” refers to the mixture of mucus secreted by the cervix, shed epithelial cells, vaginal transudate, and bacteria found in the vagina of a woman.

“Isolated bacterial strain” means a strain that has been separated from other strains (e.g., from a vaginal bacterial community, e.g., derived from a sample of cervicovaginal secretions or vaginal fluid) and cultivated in vitro in a culture comprising said strain. An isolated bacterial strain is substantially free of contaminants or components that accompany the material it was derived from in its native state (e.g., such as, vaginal mucus and epithelial cells).

“Culture-independent method” means methods not involving isolation and/or in vitro propagation of bacterial strains, e.g., in cultures.

The term “microbe” is used synonymously with the term “microorganism” and includes bacteria (Archaea, Eubacteria), yeast, fungi, and viruses. The term “species” is used herein to refer to a taxonomically and/or genetically distinct group of microorganisms. Species may include one or more distinguishable (e.g., by sequencing) strains.

The term “microbiota” refers to a community of microorganism localized to a distinct shared environment (a “microbial niche”). For example, “vaginal microbiota” is a community of one or more species of microorganisms that are localized to, or found in, a vagina. The term “microflora” or “flora” is used synonymously with the term “microbiota.” Healthy or normal microbiota denominates the community of commensal microorganisms that colonize (inhabit) a particular microbial niche of the host, such as the vagina. Bacteria are the most numerous microbial components of the normal flora.

“Mucosa” as used herein indicates a mucous membrane. Mucus is a secretion produced by, and covering, mucous membranes. Mucous fluid is viscous and typically produced from mucous cells (e.g., goblet cells) found in mucous membranes and submucosal glands, and rich in antiseptic enzymes (such as lysozyme), immunoglobulins, inorganic salts, proteins such as lactoferrin, and glycoproteins (mucins). Mucosal surfaces include epithelial linings of the reproductive tract (vagina) and, e.g., lactobacilli are capable of colonizing the vaginal mucosal surfaces.

As used herein, the term “effective amount” means the amount (e.g., of a composition or preparation) to be administered to a typical subject (e.g., a female recipient) that is sufficient to lead to a desired beneficial or therapeutic effect in the subject. The desired beneficial or therapeutic effect includes prophylaxis and/or treatment, e.g., of dysbiosis, inflammation or an infection or urogenital tract, and also includes the restoration and/or rebalancing of the vaginal microbiota (e.g., to achieve homeostasis), e.g., an anti-inflammatory and/or anti-pathogenic state of the urogenital tract and the vaginal microbiota. An effective amount, for example, is the amount sufficient (e.g., at dosages and for periods of time necessary) to alleviate at least one or more symptom, delay the development of a symptom, alter the course of a symptom (e.g., slowing the progression of a symptom), or reverse a symptom. Effective amounts generally cause statistically significant, measurable changes.

The effective amount in a delivery system varies depending on the particular agent, intended use, expected release rate and the time for which the system is expected to provide therapy. A variety of devices with varying sizes can be formulated for administering dosages. A person skilled in the art is readily able to determine the effective amount of the active agent needed for each specific application and delivery system. Effective amounts can be determined, e.g., in clinical trials and animal studies. The term “effective amount” is used interchangeably with the term “therapeutically effective amount”.

A “lyophilized” or “freeze-dried” composition refers to a composition from which moisture has been removed, e.g., for easy storage and transport. Such compositions can be rehydrated before use (e.g., administration).

As used herein, the term “viable” refers to a cell (e.g., a bacterial cell) that is able to survive in a given condition (e.g., storage for a certain period of time under particular storage conditions, e.g., including, temperature, humidity) and is generally able to colonize and reproduce (e.g., in the urogenital tract) after exposure to the condition. Percent viability refers to the percentage of viable cells in a population. For example, percent viability can refer to the percentage of lactobacilli in a pharmaceutical composition that will survive (e.g., refrigeration, freezing and/or storage) and colonize upon application to a vaginal mucosal surface.

The abbreviation “cfu” means “colony-forming unit”.

The abbreviation “VCC” means viable cell count (as determined by life cell staining).

The phrases “excipient” “pharmaceutically acceptable carrier” “diluent” or “buffer” as used herein mean a non-active, pharmaceutically acceptable material, ingredient, composition or vehicle that is added to form part of the final formulation and/or maintains a drug or other agent in a form for delivery to a subject. Each carrier preferably is compatible with the other ingredients of the formulation, for example the carrier does not decrease the impact of an active ingredient or agent upon the treatment, e.g., the carrier is pharmaceutically inert. In some embodiments a pharmaceutically acceptable carrier can be a carrier other than water (including, e.g., a cream, emulsion, gel, liposome, nanoparticle, film, ointment and/or vaginal device). In some embodiments, a pharmaceutical composition is provided comprising the substantially complete vaginal microbiota preparations together with a pharmaceutically acceptable carrier and/or diluent (e.g., saline). These compositions allow the easy administration of the preparations by means known to the person skilled in the art. In some embodiments, a buffering agent is added, e.g., a weak acid or base that maintains the acidity at a chosen level (e.g., between pH 3.5 and 4.5) and prevents a rapid change in acidity.

The terms “contacting”, “administering” or “subjecting” and more specifically, “vaginal application” or “vaginal administration”, are used interchangeably herein and relate to a subject (e.g., a female recipient) or a specific organ or other physiological site (e.g., the urogenital tract or vaginal cavity or a subpart thereof, e.g., to a site on a vaginal wall (mucosal or endometrial surfaces) or vaginal fornices) and a device, dosage form or pharmaceutical composition that is provide or given to the subject or site, e.g., for the purpose of colonizing and engrafting a desired bacterial community (e.g., comprising lactobacilli), e.g., as comprised in the substantially complete vaginal microbiota preparations described herein. In one embodiment, administering is performed locally. In one embodiment, administering is performed vaginally, e.g., to improve vaginal health, e.g., in the context of treatment of a disease or disorder.

As used herein, the terms “pharmaceutical composition” or “therapeutic composition” refer to the active agent (e.g., the substantially complete vaginal microbiota preparations described herein) in combination with a pharmaceutically acceptable carrier, e.g., a carrier commonly used in the pharmaceutical industry, or an additional active agent. Pharmaceutical compositions are physiologically and pharmacologically acceptable. Generally, compounds, materials (devices), compositions, and/or dosage forms are “pharmaceutical” “therapeutic” or “pharmaceutically acceptable” if they are, within sound medical judgment (e.g., by a physician or regulatory agency), suitable for use in contact with the tissues of human beings without excessive toxicity, irritation, allergic response, or other problem or complication, commensurate with a reasonable benefit/risk ratio (e.g., desired benefit(s) versus side effects (adverse events)). A pharmaceutical composition will generally comprise agents for buffering and preservation in storage, and can include buffers and carriers for appropriate delivery, depending on the route of administration. Pharmaceutical compositions can be conventionally administered in a unit dose. The term “unit dose” refers to physically discrete units suitable as unitary dosage for the subject, each unit containing a predetermined quantity of active material calculated to produce the desired therapeutic effect in association with the required physiologically acceptable diluent, i.e., carrier, or vehicle.

The terms “dose” and “dosage” are used interchangeably herein. A dose refers to the amount of active ingredient given to an individual at each administration. The dose will vary depending on a number of factors, including frequency of administration (e.g., daily, one or more times per week, per month, or per 3 months); size and tolerance of the individual; severity of the condition; intended result (e.g., treatment, prophylaxis, modulation or restoration of the microbial community), and the route of administration. A baseline dose can be administered and modified based on the initial response of the subject. For example, a single dose of the compositions comprising the substantially complete vaginal microbiota preparations described herein can be in the range of 10⁴ to 10¹⁰ colony forming units (per dose). In other embodiments, a single dose of the composition can be in the range of 10³ to 10¹², 10⁴ to 10⁹, 10⁵ to 10⁹, 10⁵ to 10⁸, 10⁶ to 10⁹, 10⁷ to 10⁹, or 10⁷ to 10¹⁰ colony forming units (per dose).

A “dosage form” refers to a particular physical form of a pharmaceutical composition and depends, e.g., on the dose required to deliver a desired amount of an active agent and on the route of administration. For example, a dosage form can be in a suppository, a tablet, a capsule, a film, a cream, etc., or a device, such as, e.g., an applicator or dispenser, e.g., for vaginal administration. Dosage forms may be single or multiple-use dosage forms.

As used herein, the terms “treat,” “treatment,” “treating” refer to prophylactic and therapeutic treatments, wherein the object is to prevent, reverse, alleviate, ameliorate, inhibit, slow down or stop the progression or severity of a condition or symptom of a condition associated with a disease or disorder. Treatment includes the improvement of symptoms or markers (of the disease or condition) and the cessation or at least slowing of progress or worsening of symptoms that would be expected in absence of treatment. Treatment effectiveness can be measured by monitoring one or more symptoms or clinical markers and is compared, e.g., to the subject's condition and symptoms before administration or to a control subject not undergoing treatment. In one example, treating a vaginal infection refers to reducing the amount of the infective agent (e.g., number of cells or viral particles), reducing the severity of symptoms, and/or reducing the frequency of symptoms. A treatment that reduces the level of a pathogen to one which is kept in check by the immune system or by the state established by a healthy vaginal microbiota (e.g., the infection is no longer detectable, e.g., by symptoms or general diagnostic techniques) is considered effective as the term is used herein.

As used herein, the term “colonization” or “engraftment” refers to the colonization of an environment (e.g., a microbial niche), e.g., the vagina or vaginal epithelium, by a microbe, e.g., a bacterium (e.g., lactobacilli), such that the viable population of that microbe continues to reside, e.g., in the niche, for a certain period of time. Engraftment can be transient or stable depending on the period of time the microbe continues to reside in the niche. Colonization and engraftment (and residence time) can be quantified, e.g., by counting the number of colony forming units (CFU)/gram and/or performing nucleic acid sequencing of microbes comprised in one or more vaginal samples that are taken over a certain period of time.

The term “subject” refers to a human (e.g., a human female) subjected to a treatment, observation or study. In one embodiment, the subject is in puberty. In one embodiment, the subject is pregnant. In one embodiment, the subject is of childbearing age. In one embodiment, the subject is pre-menopausal. In one embodiment, the subject is post-menopausal. In one embodiment, the subject is a recipient of a composition comprising the substantially complete vaginal microbiota preparation described herein, e.g., a recipient female. In one embodiment, the subject is a donor female providing a microbial sample. In some embodiments, a subject is a human female suffering from or exhibiting a clinical condition related to a microbial imbalance (e.g., a dysbiosis, an infection, or an inflammatory condition). In some embodiments, a subject is a human female using the compositions and preparations described herein prophylactically.

The terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms “lower”, “reduced”, “reduction” or “decrease”, “down-regulate” or “inhibit” mean a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level (or levels below the limit of detection) as compared to a reference sample), or any decrease between 10-100% as compared to a reference level.

The term “substantially” means to a great or significant extent. For example in the case of a sample that is “substantially free of” pathogens, the term means the sample is, for the most part, or essentially, but possibly not completely, void of a pathogen. A sample that is substantially free of selected pathogens can refer to a sample that comprises about 5%, <5%, <4%, <3%, <2%, or <1% of the selected pathogens. In a particular embodiment, the term “substantially free” comprises <5%

The term “inflammation”, “inflammatory disorder”, “diseases associated with inflammation”, “conditions associated with inflammation” are used interchangeably and describe any condition, disorder, disease or state that comprise or are characterized by inflammation. The inflammation may be an acute inflammation or a chronic inflammation.

### EXAMPLES

### Example 1: Screening of Women to Identify Donors and Recipients

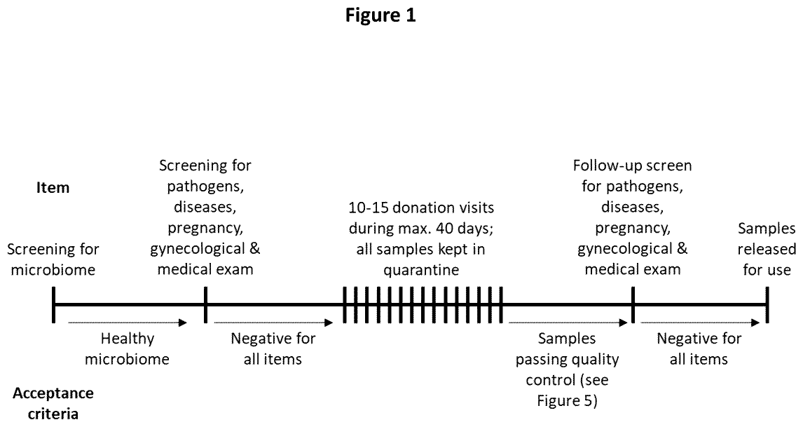

This Example describes the process of identifying suitable donors based on the analysis of the vaginal microbiome composition and absence of pathogens. A schematic overview of the screening is shown in FIG. 1.

Participant Enrollment and Vaginal Samples

Healthy women without a history of vaginal health issues were recruited. Enrolling participants were informed, consented and provided a vaginal swab for microbiome analysis, and a separate swab for HPV analysis. A total of 96 women consented and provided samples. For the last 60 women screened, instead of a vaginal swab, a cervicovaginal secretion (CVS) sample was obtained using a vaginal self-sampling device (e.g., a pliable menstrual cup that is inserted into the vaginal cavity, such as a Softdisc menstrual cup by The Flex Company, also sold under the name ‘Flex disc’) to enable both microbiome and biomarker analysis, as well as a dedicated swab for HPV screening. Both sample types were obtained via self-sampling by donors after thorough instruction of the donor on the correct sampling procedure.

Microbiome Analysis

The microbiome composition from the vaginal swab or CVS samples was determined by shotgun DNA sequencing analysis. Swabs for vaginal microbiome analysis were kept at 4° C. for up to 48 hours prior to DNA extraction. CVS samples were diluted with saline to reduce the viscosity, aliquoted (for procedure, see Example 3) and stored at −80° C. prior to DNA extraction. DNA was extracted from both sample types using the Molysis Complete5 kit (MolZym), which uses a differential lysis method to extract microbial DNA and remove human DNA. Shotgun sequencing and bioinformatics analyses of the microbiomes was performed by Seqbiome (Cork, IE). All samples were prepared for shotgun metagenomic sequencing according to Illumina Nextera XT library preparation kit guidelines, with the use of unique dual indexes for multiplexing with the Nextera XT index kit (Illumina). Samples were sequenced on an Illumina NextSeq 500 sequencing platform with a v2.5 kit (a 300-cycle kit/150 bp PE sequencing), at the Teagasc DNA Sequencing Facility, using standard Illumina sequencing protocols. Quality of the raw sequencing data was assessed using FastQC. Samples that did not meet minimum quality check cutoff or a sequencing depth of at least 4M reads underwent re-sequencing. Shotgun metagenomic sequencing data were then processed through analysis workflow that utilizes Kneaddata wrapper tool. Quality filtering and host genome decontamination (human) were performed utilizing Trimmomatic and Bowtie2. Taxonomic classification of quality filtered reads was further performed using Kraken2 species classifier using a customized version of Genome Taxonomy Database (GTDB) that also includes reference sequences belonging to archaea, fungi and viral genomes. Kraken2 classifications were then passed to Bracken tool to estimate species level abundance. The vaginal microbiome composition was determined for each putative donor and recipient by obtaining information regarding the presence and relative abundance of bacterial species.

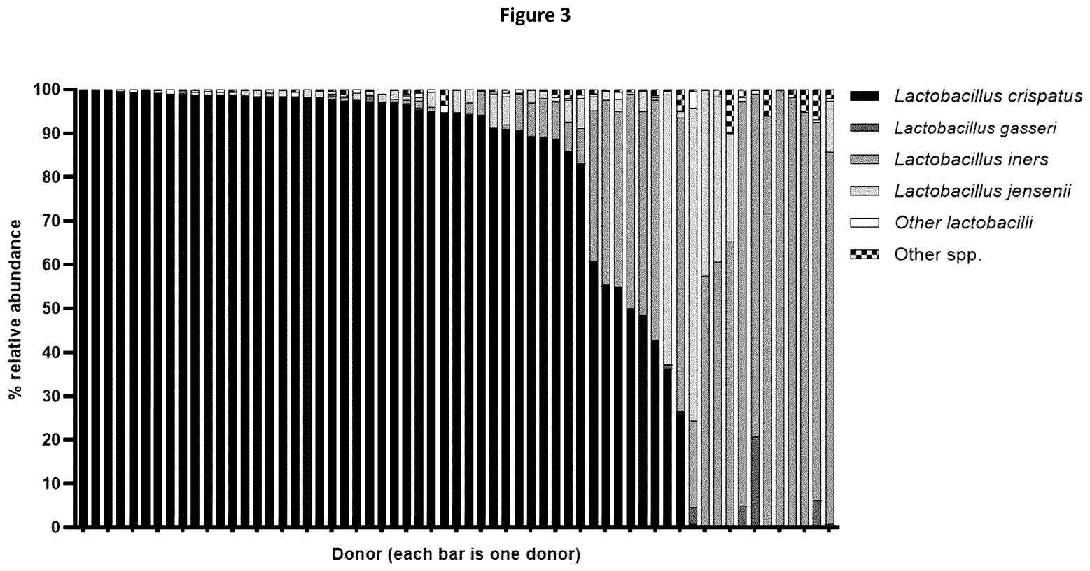

For this study, to qualify as a donor, the vaginal microbiome composition had to pass two criteria: to comprise at least 80% vaginal lactobacilli (L. crispatus, L. iners, L. jensenii, L. gasseri) and to comprise less than 5% selected pathogens (Atopobium spp., Prevotella spp., B. vaginale, and F. vaginae). The vaginal microbiome composition of 61 out of 96 women (64%) satisfied these two criteria and were considered healthy vaginal microbiomes. The relative abundance of the lactobacilli present in the vaginal microbiota are depicted in FIG. 3, wherein each bar represents the microbiome composition of a single healthy donor. The relative quantities of species are indicated in the y-axis. The microbiomes of the screened donors differed in their relative composition. In several instances, the microbiome was dominated by a single species, e.g., L. crispatus, wherein other microbiomes showed a heterogenous population of lactobacilli, e.g., comprising a combination of L. crispatus, L. iners and L. jensenii. Of note, even minute relative quantities, such as less than 0.05% could be detected (see, Table 2).

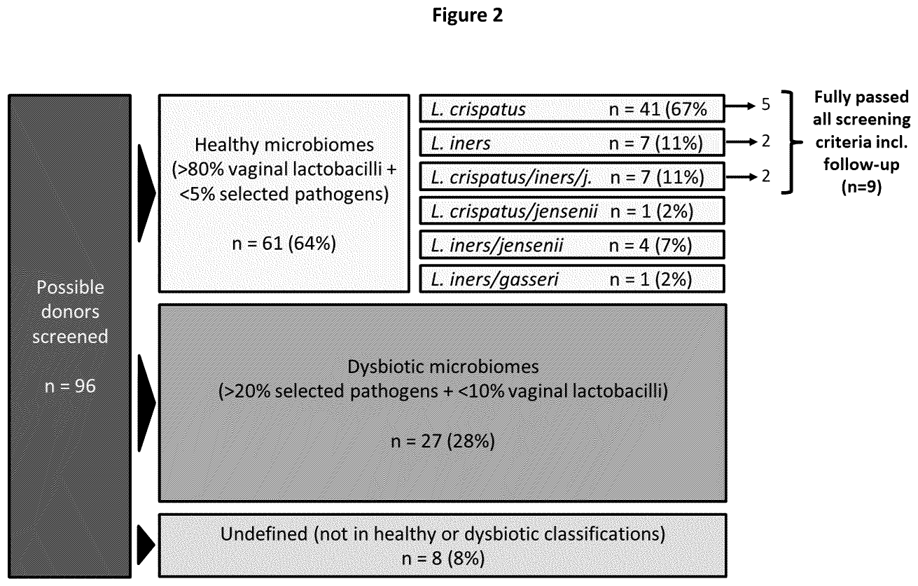

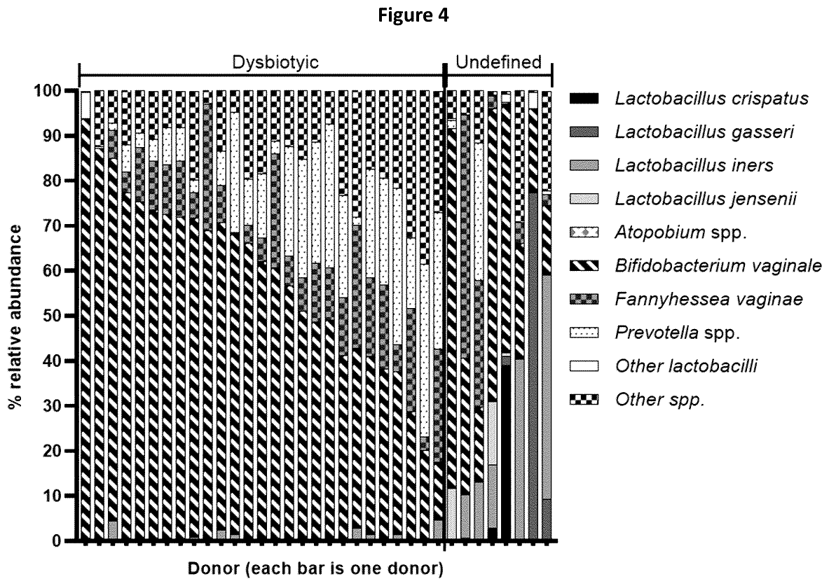

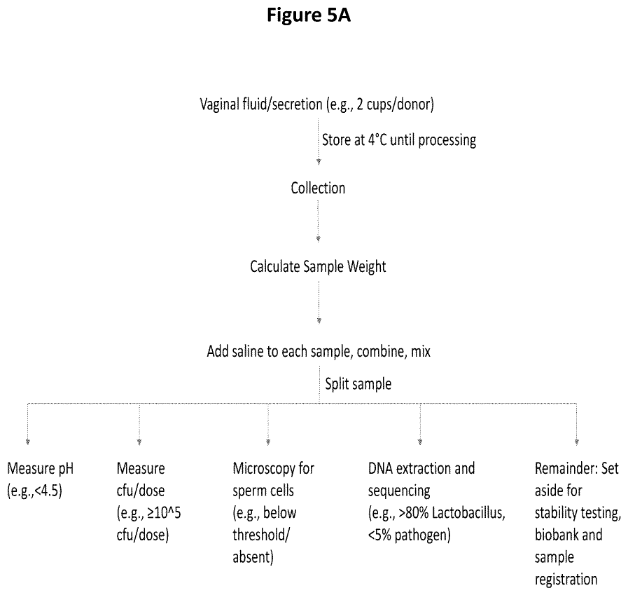

Of the 96 women screened, 28% (n=27) exhibited a dysbiotic microbiome (defined as >20% selected pathogens and <10% vaginal lactobacilli) (see, FIGS. 2 and 4). The women that exhibited dysbiotic vaginal microbiota were free of vaginal disease symptoms and of general good health. The remaining 8% (n=8) fell between the healthy and dysbiotic microbiome classifications, did not exhibit any vaginal disease symptoms, or contained species not classified/defined in the screening criteria (FIG. 4). These women did not qualify as putative donors or recipients.

The microbiome compositions of the healthy cohort (n=61) had a median vaginal lactobacilli abundance of 99.26% and selected pathogens of 0.03%. In contrast, the dysbiotic cohort (n=27) was characterized by a median vaginal lactobacilli abundance of 0.34% and selected pathogens of 87.21%. The undefined cohort (n=8) was characterized by a median vaginal Lactobacilli abundance of 35.76% and selected pathogens of 61.83%.

This suggests that the selection criteria (see, FIG. 2) can reliably distinguish healthy and dysbiotic vaginal microbiota with high certainty. Only healthy vaginal microbiomes were considered as putative donors and underwent further stringent screening procedures.

HPV Screening

Human papillomavirus (HPV) is a highly prevalent viral infection, several strains of which are associated with cervical cancer. The prevalence of HPV-positive women is high. On the vaginal swabs taken for HPV screening during the donor's screening visit, DNA extraction was performed using the Qiagen QIAmp DNA mini kit. Extracted DNA from each donor sample was used in the SeeGene Anyplex II HPV28 kit and ran semi-quantitatively on the BioRad CFX96 Dx qPCR machine, which was set up and calibrated for this assay (Triolab, DK). The semi-quantitative method provides three levels of quantity for 28 different genotypes of HPV, which included all known high-risk genotypes. Only samples that were HPV-negative were considered suitable putative donors.

Pathogen Check and Examination by Medical Staff

To minimize any adverse effects for the recipients, putative donors (women who had a healthy microbiome and passed the HPV screen) were further screened by a gynecologist for the presence of pathogens, pathobionts, and sexually transmitted diseases, and underwent a medical and gynecological examination to assess the presence of other diseases such as cancer or endometriosis. The procedure included an additional HPV test.

Standard diagnostic tests were performed for HIV, Hepatitis A, B, and C, cytomegalovirus, Treponema, urinary tract infections, HPV, chlamydia, gonorrhea, Trichomonas, Herpes genitalis, Candida, Mycoplasma, and Streptococcus A, B, C, and G. During the medical examination, putative donors were subjected to a general health check including medical history and medication usage, demographics, heart rate and blood pressure measurements.

For approved donors, the same tests were again performed at a follow-up visit after they had provided their last donations over a specified time period (see, FIG. 1). Until approved donors passed the test at the follow-up visit, and all samples passed all quality checks (see, Example 4), the samples remained quarantined and not released for administration to a recipient.

Donor Selection

Donors were selected on the screening procedure described above, including of microbiome analysis, pathogen screening, and medical and gynecological examination. Only subjects that had a healthy vaginal microbiome, had no positive pathogen test, and had no abnormal findings in the medical and gynecological exam were considered suitable donors and were enrolled in the program (see also, Example 2).

Following the screening protocol, 12 (13%) out of 96 screened women were enrolled into the donor program and started their donation visits. Of these, three failed to provide sufficient donations or failed during follow-up screening. Overall, of all 96 women who enrolled for screening 9% fully passed all screening criteria resulting in nine approved donors (n=9) (Table 1).

Table 1 provides an overview of the pathogens and pathobionts that were screened for in donor subjects (n=38) that had passed HPV screening.

TABLE 1

| | n positive |
|---|---|
| Screening | out of n = 38 |
| HIV | 0 |
| Hepatitis A/B/C | 0 |
| CMV IgM | 0 |
| Treponema | 0 |
| UTI | 2 |
| HPV | 13 |
| Chlamydia | 1 |
| Gonorrhoeae | 0 |
| Trichomonas | 0 |
| Herpes genitalis | 0 |
| Mycoplasma | 1 |
| Strep A/B/C/G | 4 |
| Candida | 7 |
| Other (e.g., abnormal gynecological findings) | 4 |
| Total passed - out of the original 96 women | 12 |
| Failed during donations or at follow-up | 3 |
| Total fully passed/approved donors | 9 |

The composition of the substantially complete vaginal microbiota preparation of all screened subjects (n=96) including the fully approved donors (n=9) is summarized in Table 2.

Table 2 Vaginal microbiota compositions of four cohorts: (i) Healthy, (ii) fully approved donors, (iii) dysbiotic and (iv) undefined. Shown are the relative amounts of the quantified bacteria in median and interquartile range (IQR) (in %) relative to the total amount of detectable species in the sample preparation.

TABLE 2

| | Healthy | | Fully approved | | Dysbiotic | | Undefined | |
|---|---|---|---|---|---|---|---|---|
| | (n = 61) | | donors (n = 9) | | (n = 27) | | (n = 8) | |
| Species | median | IQR | median | IQR | median | IQR | median | IQR |
| Lactobacillus | 94.75 | 50.05 | 97.42 | 50.16 | 0.00 | 0.00 | 0.03 | 1.08 |
| crispatus | | | | | | | | |
| Lactobacillus gasseri | 0.00 | 0.08 | 0.00 | 0.00 | 0.00 | 0.00 | 0.28 | 3.84 |
| Lactobacillus iners | 0.84 | 42.20 | 0.84 | 46.48 | 0.34 | 1.11 | 11.47 | 20.79 |
| Lactobacillus jensenii | 0.74 | 2.82 | 0.07 | 1.34 | 0.00 | 0.00 | 0.10 | 3.42 |
| Total vaginal | 99.26 | 1.44 | 99.62 | 0.37 | 0.34 | 1.10 | 35.76 | 33.35 |
| lactobacilli | | | | | | | | |
| Atopobium spp. | 0.00 | 0.00 | 0.00 | 0.00 | 0.00 | 0.01 | 0.00 | 0.00 |
| Bifidobacterium | 0.01 | 0.30 | 0.00 | 0.08 | 61.60 | 31.71 | 27.98 | 39.65 |
| vaginale | | | | | | | | |
| Fannyhessea vaginae | 0.00 | 0.00 | 0.00 | 0.00 | 10.70 | 9.75 | 2.63 | 10.11 |
| Prevotella spp. | 0.00 | 0.01 | 0.00 | 0.00 | 10.23 | 22.19 | 0.02 | 0.46 |
| Total selected | 0.03 | 0.50 | 0.01 | 0.08 | 87.21 | 10.10 | 61.83 | 49.59 |
| pathogens | | | | | | | | |
| Other lactobacilli | 0.31 | 0.55 | 0.15 | 0.14 | 0.00 | 0.00 | 0.28 | 0.98 |
| Other species | 0.14 | 0.58 | 0.03 | 0.33 | 12.48 | 10.96 | 5.71 | 13.50 |

“Healthy”, “Dysbiotic” and “Undefined” cohorts were categorized based on the relative quantities of the vaginal lactobacilli and pathogens as described above (see also, FIG. 2). “Fully approved donors” refers to subjects that, in addition to passing the first screen successfully underwent additional screens, e.g., to exclude STIs and other infections and medical conditions as described above. Subjects that passed all screening criteria and qualified as suitable donors (n=9) had a median vaginal lactobacilli concentration of 99.62% and selected pathogens of 0.01%. Approved donors had the highest amount of selected vaginal lactobacilli and the lowest amount of vaginal pathogens of the groups. Fully approved donors were not limited to L. crispatus-dominant vaginal microbiota compositions but comprised a mixture of lactobacilli species or were L. iners dominant.

Recipient Selection

Recipients were recruited and enrolled in clinical studies. In one study, healthy volunteer women aged 18 to 45 were recruited for screening and participation. Only women without vaginal disease symptoms and of general good health were enrolled for screening. Another study enrolled a single patient with vaginal symptoms. All recipients were classified as having vaginal dysbiosis (harboring a dysbiotic vaginal microbiome) according to the inclusion criteria defined above (see, FIG. 2): >20% selected pathogens (Atopobium spp., Prevotella spp., B. vaginale, and F. vaginae) and <10% vaginal Lactobacilli.

### Example 2: Obtaining Donor CVS Samples

This Example describes the process of obtaining vaginal microbiota samples from a donor, wherein the sample comprises substantially complete vaginal microbiota.

Donation Visit Setup

Donors that successfully passed the screening process described in Example 1 were invited to donate cervicovaginal secretions (CVS) within a time period of 40 days between the two gynecologist/pathogen check visits (see, FIG. 1). Each donor provided 10-15 donations at any time during the 40 days except on days during menstruation and one day thereafter. The donations were spaced at least 16 hours apart.

During the donation period, donors needed to adhere to the following restrictions: abstinence from vaginal and anal sexual intercourse; no swimming in lakes, Jacuzzis and swimming pools; no use of intravaginal products (e.g., tampons, soap). At each visit, the donor filled out a questionnaire to affirm adherence to these restrictions together with general health questions.

After the donation period, the donors underwent a follow-up check at the gynecologist. Only if they passed this follow-up, were their samples considered for release and use for administration (see, FIG. 1, Example 1). Until that time, and until all samples passed all quality checks (see, Example 4), all samples were kept in quarantine.

Donor Self-Collection of CVS

Cervicovaginal secretion (CVS) samples were obtained through self-collection using a vaginal self-sampling device after thorough instructions. Donors obtained CVS samples in a dedicated, hygienically designed donor room. The donor room was cleaned with 70% ethanol after each donor and subjected to biweekly environmental monitoring to check for Enterobacteriaceae, total aerobic microbial count, and yeast and mold. This setup maximizes cleanliness and minimizes minimized processing time, compared to, e.g., home sampling. Only a single contamination was detected in one of the donor samples consisting of skin bacteria, caused by the donor not following the sampling procedure correctly.

The vaginal self-sampling device was a single use menstrual cup with a flexible/pliable ring and plastic foil cup (described in Example 1). For CVS donations, it was not worn like a menstrual cup over the cervix, but instead used as a large swab by inserting it partially folded into the vagina, leaving it in a longitudinal position for about 10 seconds and twisting it along its longitudinal axis while removing it. The donor deposited the vaginal self-sampling device into a provided labeled and pre-weighed sterile 50 mL tube. After 15-20 min, this process was repeated a second time with a new vaginal self-sampling device, which was placed in a separate sterile 50 mL tube.

The soft and pliable material of the vaginal self-sampling device in conjunction with it being inserted partially folded into the vaginal canal enables the effective collection of CVS from the surface of the vaginal cavity, without damaging or irritating the vagina. The vaginal self-sampling devices are intended and safe for vaginal use, but to ensure maximum donor safety and control, each batch of the devices was screened for contamination of Enterobacteriaceae and confirmed to be free of any contamination.

The procedure described here is believed to have several advantages over using the vaginal self-sampling device over a prolonged time period as a collecting device over the cervix. With the above method, the bacteria comprised in the CVS sample will spent less time in contact with the vaginal self-sampling device, and the amount of vaginal mucus and vaginal bacteria that is collected is increased. The vaginal epithelium contains the substrates for the vaginal microbiome. Thus, a sample obtained in this manner has a higher concentration of viable vaginal lactobacilli and mucus, and a lower amount of fluid from the endometrium.

### Example 3: Producing a Substantially Complete Vaginal Microbiota Preparation

This Example demonstrates the production of a substantially complete vaginal microbiota preparation from cervicovaginal secretions.

Process to Produce a Substantially Complete Vaginal Microbiota Preparation

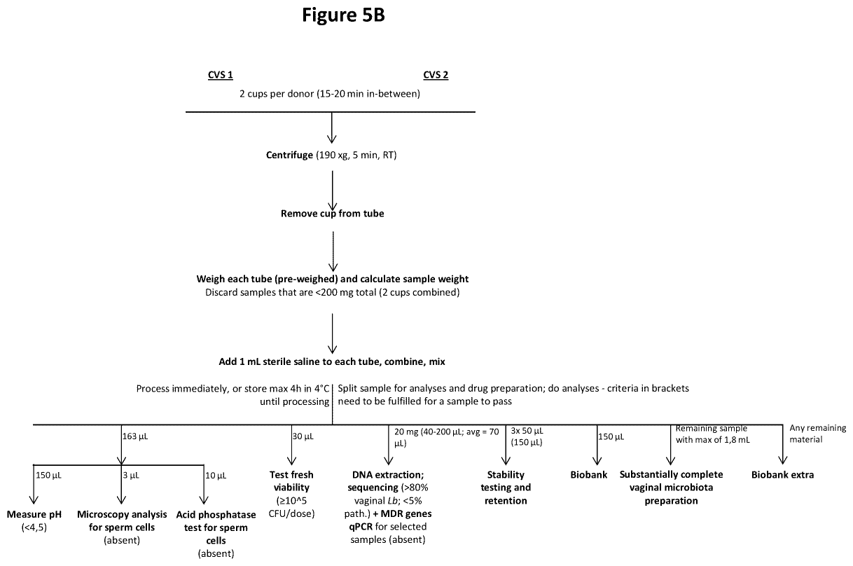

A schematic representation of the sample processing is provided in FIGS. 5A and 5B. At each visit, the donor was provided with two 50 mL sterile tubes, which were pre-labelled and pre-weighed, and two vaginal self-sampling devices, along with the questionnaire (see, Examples 1 and 2). The CVS was collected by centrifuging for 5 min at 190×g and ambient temperature. The low speed collects the CVS from the device, while not phase-separating into layers. All further steps were conducted under sterile conditions at ambient temperature. The self-sampling devices were discarded from the tubes and the CVS sample weight was determined. Samples with a total (two tubes combined) weight of less than 200 mg were discarded. Samples in which blood was visibly present were discarded.

The sample in each tube was mixed with 1 mL saline to reduce viscosity, after which the two samples were combined and aliquoted for storage and further testing. The two combined samples were then distributed over several aliquots for quality control (see, Example 4) and storage as shown in FIG. 5B.

The cryovials containing samples for storage were placed in a CoolCell (Corning) and then at −80° C. A CoolCell has a controlled cooling rate of 1° C. per minute, which ensures maintaining maximum viability of the samples. After a minimum of 2 hours, the samples were transferred into a regular −80° C. storage box labelled ‘quarantined’. Samples were released only after all release criteria had been met (see, FIG. 5B and as described above).

### Example 4: Quality Control

This Example describes the quality control of a substantially complete vaginal microbiota preparation produced from cervicovaginal secretions, so it can be used safely for administration.

Overall Procedure

A schematic representation of the sample processing procedure with details about quality control is provided in FIGS. 5A and 5B. This procedure is highly optimized, in such a way that the volumes used for analyses and quality control are as low as possible to minimize loss of substantially complete vaginal microbiota preparation. The sample used for pH measurement is discarded after measurement. The retention vial is maintained for at least 1 year after administration for safety reasons (e.g., to check for STIs in case one occurred in a recipient). Every first and last sample of each donor was subjected to a qPCR analysis to check for the absence of multi-drug resistance genes (MDR genes).

Analyses for Quality Control

The pH was measured using a micro-pH probe. Technical triplicate measurements were performed, and repeated if the difference between measurements was >0.2. The sample used for pH measurement is discarded after measuring. Microscopy was performed. The absence of sperm cells was determined optically by microscopy and using acid phosphate paper, a highly sensitive and selective method to identify sperm cells. Viability was tested by colony-forming units (CFU) count on de Man, Rogosa and Sharpe (MRS) agar plates or with viable cell count (VCC) for donors who at screening were dominant in L. crispatus, L. jensenii, or L. gasseri. For donors who at screening were dominant for L. iners, this method could not be used. Instead, viability for these samples was tested using BacLight live/dead staining (ThermoFisher Scientific) and counting the viable cells/mL in a Thoma cell counting chamber under the microscope. Within one week after a sample was used for administration, a stability vial was used to test cell viability at the time of vaginal administration, and dose calculation.

DNA extraction was performed using the Molysis Complete5 kit (Molzym) according to the manufacturer's instructions to obtain sufficient bacterial sequence reads to perform in silico engraftment check after administration based on metagenome data (see, Example 1 for methods used). The multi-drug resistance (MDR) marker qPCR was performed using the same DNA as was used for Shotgun sequencing, using the SeeGene Allplex Entero-DR qPCR assay kit on a BioRad CFX96 Dx qPCR machine calibrated for the assay. This kit allows single or multiple detection of carbapenemase genes (NDM, KPC, OXA-48, VIM, IMP), extended spectrum beta-lactamase (ESBL) genes (CTX-M), and vancomycin resistance genes (VanA, VanB).

### Example 5: Donor Microbiome Characterization

This Example describes the substantially complete vaginal microbiota preparations suitable for vaginal administration.

Microbiome Compositions During the Donation Period

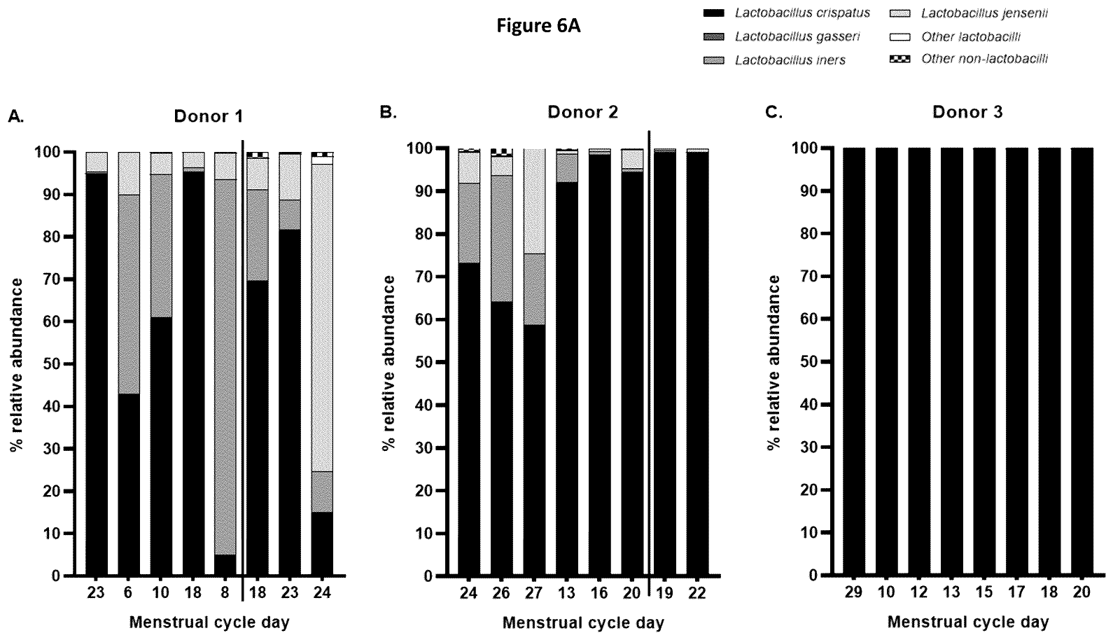

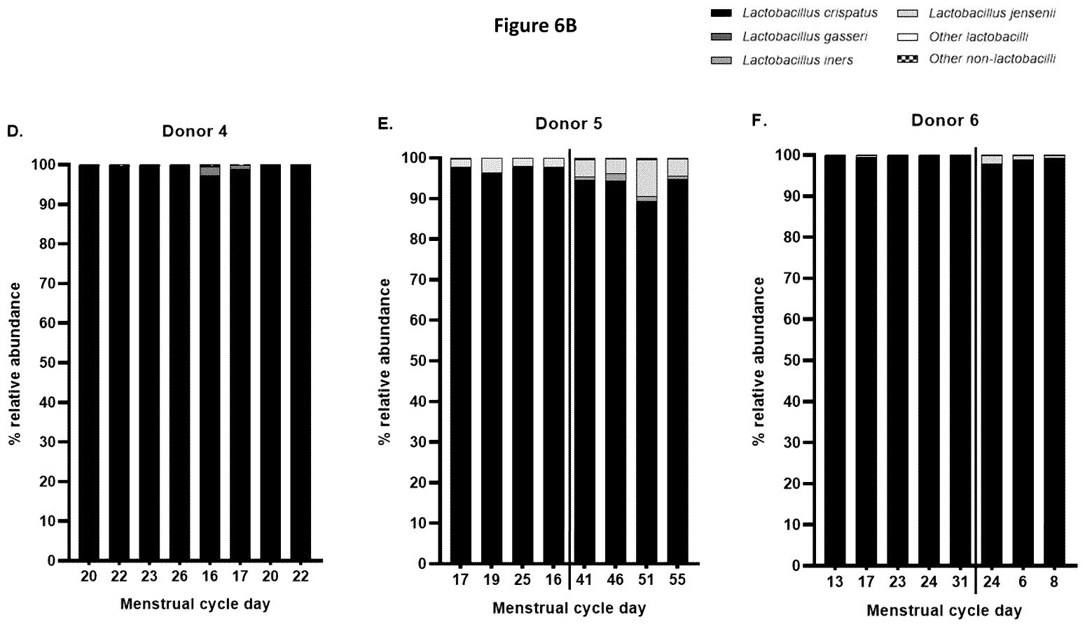

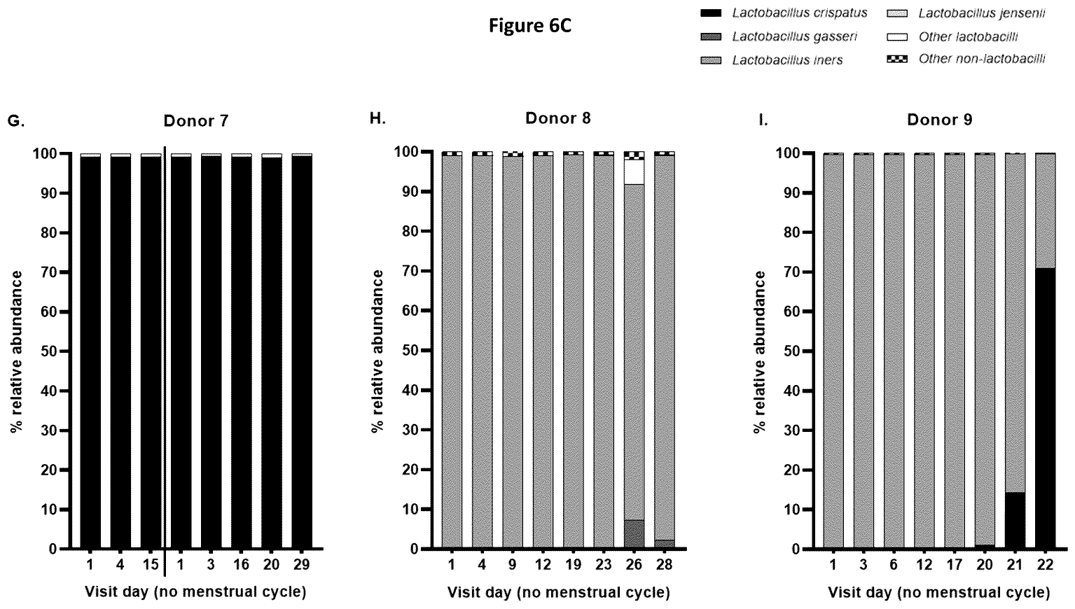

It is known that the vaginal microbiome in women can fluctuate over the menstrual cycle or depending on sexual activity (Gajer et al., 2012, Science Translational Medicine). For donors with mixed species compositions that did not use contraception (Table 3), fluctuations between the species were observed over the menstrual cycle (FIG. 6A, B), but the totality of L. crispatus, L. iners, L. jensenii, L. gasseri in the sample remained stably>80%. The microbiome of donors with a strongly L. crispatus-dominant microbiome was very stable over extended periods of time (e.g., Donor 7, FIG. 6 G).

The microbiome of L. iners-dominant donors was mostly stable, although in one case a shift between L. iners and L. crispatus was observed towards the end of the donation period (FIG. 6 I). This donor did not have a menstrual cycle due to due contraception. The microbiome stability observed in the donors is likely aided by the restrictions that the donors adhere to, such as, e.g., no sexual intercourse.

Some donors returned for a second round of donation visits. In FIG. 6, this is indicated by a vertical line. There was a 1 month in-between two rounds for donors 2, 5, and 7, and a 1.5-months for donor 1. For donor 6, there was a 2-months in-between the rounds, and she changed from using contraceptive pills in the first round to no hormonal contraceptives in the second round.

In several donor samples, a certain species (mostly L. jensenii) was present in small but very consistent amounts (0.3-3.5%). In one of these donors, an even smaller amount of L. iners was consistently present (0.07-0.33%). Even smaller amounts of both L. iners (0.02-0.10%) and L. jensenii (0.01-0.03%) were consistently observed in yet another L. crispatus-dominant donor. In the two donors with mixed species composition, none of the species present in the mixture fully disappeared (below detection level) over the donation period even though their relative abundances sometimes decreased to as low as 0.27%. Altogether, this implies that the vaginal microbiome is a stable ecosystem and points to a symbiotic relationship between the different Lactobacillus species in one person. This is further substantiated by the observations of the vaginal administration of the preparations, as described in Example 7.

In addition to the four main vaginal lactobacilli (L. crispatus, L. iners, L. jensenii, L. gasseri), other lactobacilli were regularly observed to be consistently present in small amounts in donor samples (mostly <0.05%): L. acetotolerans, L. acidophilus, L. amylovorus, L. gallinarum, L. gigeriorum, L. helveticus, L. johnsonii, L. kefiranofaciens, L. kitasatonis, L. paragasseri, L. psittaci, Lactobacillus sp002911475, L. taiwanensis, L. ultunensis, L. coleohominis, L. reuteri, and L. vaginalis. The stable presence of these lactobacilli further points to a complex ecosystem that in its entirety might be needed to promote and maintain a healthy vaginal niche.

Some other lactobacilli appeared sporadically in a subset of the donor samples. L. helveticus was not observed in the two L. iners-dominant donors, and only appeared in the last visit of a single donor who shifted from L. iners-dominant to L. crispatus-dominant, which may suggest that certain species prefer cohabitation with each other while others might be incompatible in the ecosystem. In contrast to reports in the literature where L. iners-dominant donors were not considered suitable for administration, the data suggest that L. iners-dominant microbiomes of fully approved donors have a very stable and healthy ecosystem (see also inflammatory marker data in Example 8) and are suitable donors for substantially complete vaginal microbiota preparations.

L. gasseri was infrequently observed as a dominant species (FIG. 3) but was typically found in small amounts in several of the donors (FIG. 6, Table 1). L. gasseri was detected in L. crispatus-dominant donor 4 (FIG. 6 D) in relative concentrations of 0.01-2.23%; in mixed microbiome donor 1 (FIG. 6A) in relative concentrations of 0.01-0.07%; and in L. iners-dominant donor 8 (FIG. 6 H) in relative concentrations of 0.3-7.14%. The consistent maintenance of minor Lactobacillus species suggests that the minor species might play a hitherto unknown role in maintenance or stability of the vaginal ecosystem.

Physical Properties of the Obtained Samples

The weight of samples fluctuated among donations of the same donor and among donors, but on average was >200 mg. CFU and pH remained stable and within the defined threshold of <4.5 (Table 3). Altogether, this stability over both the microbiome composition and sample properties indicates the health and stability of the donor microbiomes.

Table 3 summarizes the properties of the CVS and substantially complete vaginal microbiota preparations that are generated. Each row contains data for one donor during her 10-15 donations over a 40-day period. The ‘Time window’ column lists the number of days during which the donor provided the number of samples listed in the ‘Nr of donations’ column. Donor ID is the same as in the microbiome graphs in FIG. 6. The weight is the combined weight of the two samples provided by the donor, prior to adding saline. The dose is the final amount of mL in the substantially complete microbiome preparation vial used for dosing a recipient, after all volumes for analyses have been taken out (see, FIG. 5 B). The maximum dose is 1.8 mL to fit in a cryovial. Viable cells/dose was determined by CFU plate counting on MRS medium grown anaerobically for 2-3 days for all species except for the two L. iners-dominant donors (8 and 9). For these, viable cells/dose was determined by live/dead staining and counting viable cells in a counting chamber under the microscope. Abbreviations: hIUD: hormonal intrauterine device; pill: oral contraceptive.

TABLE 3

| | | | | Time | Ranges (min-max) | | | | Averages | | | |
|---|---|---|---|---|---|---|---|---|---|---|---|---|
| Donor | | Contra- | No of | window | | Dose | | Viable | Weight | Dose | | Viable |
| ID | Microbiome | ception | donations | (days) | Weight (mg) | (mL) | pH | cells/dose | (mg) | (mL) | pH | cells/dose |
| 1 | Mixed | none | 15 | 28 | 244-1035 | 1.5-1.8 | 3.9-4.4 | 8.6E+06- | 515 | 1.6 | 4.1 | 1.2E+08 |
| | | | | | | | | 3.1E+08 | | | | |
| 2 | Mixed | none | 10 | 36 | 311-918 | 1.4-1.8 | 4.1-4.3 | 1.9E+08- | 597 | 1.7 | 4.2 | 3.6E+08 |
| | | | | | | | | 5.4E+08 | | | | |
| 3 | L. crispatus | none | 11 | 25 | 628-1778 | 1.7-1.8 | 3.7-3.8 | 5.1E+07- | 1206 | 1.8 | 3.8 | 1.5E+10 |
| | | | | | | | | 8.4E+10 | | | | |
| 4 | L. crispatus (+ | hIUD | 14 | 31 | 195-486 | 1.3-1.7 | 3.9-4.2 | 7.2E+07- | 334 | 1.5 | 4.1 | 1.9E+08 |
| | some L. gasseri) | | | | | | | 3.4E+08 | | | | |
| 5 | L. crispatus + | pill | 12 | 30 | 340-823 | 1.5-1.8 | 3.7-4.0 | 3.3E+08- | 525 | 1.7 | 3.9 | 7.4E+09 |
| | L. jensenii | | | | | | | 1.0E+11 | | | | |
| 6 | L. crispatus + | pill | 14 | 20 | 276-703 | 1.3-1.8 | 3.8-3.9 | 5.0E+07- | 487 | 1.6 | 3.8 | 1.7E+10 |
| | L. jensenii | | | | | | | 1.0E+11 | | | | |
| 7 | L. crispatus + other | pill | 13 | 16 | 376-710 | 1.6-1.8 | 3.8-4.0 | 3.6E+08- | 542 | 1.7 | 3.9 | 8.5E+09 |
| | lactobacilli | | | | | | | 1.0E+11 | | | | |
| 8 | L. iners (+L. | hIUD | 12 | 31 | 532-1593 | 1.6-1.8 | 3.9-4.0 | 4.3E+08- | 868 | 1.8 | 3.9 | 8.5E+08 |
| | gasseri towards | | | | | | | 1.2E+09 | | | | |
| | end) | | | | | | | | | | | |
| 9 | L. iners (+L. | hIUD | 14 | 24 | 233-627 | 1.4-1.8 | 3.9-4.2 | 1.3E+08- | 446 | 1.6 | 4.0 | 3.0E+08 |
| | crispatus towards | | | | | | | 6.0E+08 | | | | |
| | end) | | | | | | | | | | | |
| Average | | | 13 | 27 | | | | | 614 | 1.6 | 4.0 | 5.5E+09 |

It was further evaluated whether repeated CVS donations would affect the composition of the sample. Donors provided two samples 2× per day with a 6-hour interval and 3× per day with a 4-hour interval. Both of the repeated CVS donation procedures resulted in a significant increase of the pH (approx. 0.4 increase), and a decrease of sample weight (to around or below 200 mg) and CFU (1 log), rendering repeated sampling within a single day unsuitable. This suggests that the microbiome requires more than 6 hours to fully regenerate after sampling. Consequently, donors were allowed to give samples once a day at most with approximately 16-hour intervals between donations.

Stability of the Substantially Complete Vaginal Microbiota Preparation

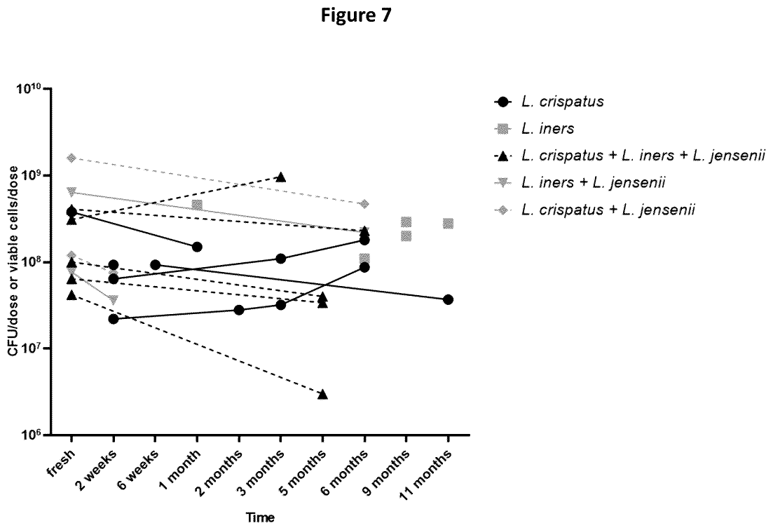

The stability of substantially complete vaginal microbiota preparations comprising different vaginal microbiome compositions was tested after different storage periods between 2 weeks to 11 months (FIG. 7). Stability was tested on 50 μL aliquots, which was found to be representative for the full volume vials (data not shown).

Generally, viability remained high over time for all species with no significant loss observed (average loss of viability below 1-log). No major differences in viability between species were observed. Other researchers have reported 2-log viability decreases after 9 months for vaginal microbiota compositions that are not L. crispatus-dominant. The improved stability seen here might, at least in part, be due to the collection method of obtaining the cervicovaginal secretion provided herein, which does not rely on a prolonged time period the collection cup is worn inside the donor subject but instead exposes vaginal mucus and bacteria only briefly to the collection cup. Without being bound by theory, it is believed that the longer the lactobacilli reside in the menstrual cup without contact or being able to adhere to the vaginal epithelium, the worse the viability, which might be particularly relevant for sensitive species, such as L. iners.

The process of obtaining the samples and processing the same described herein provides a substantially complete vaginal microbiota preparation that is stable over extended periods of time for all tested species compositions.

Further, it was assessed whether the mode of thawing the samples affected viability of the lactobacilli comprised in the preparation. When performing vaginal administration of the preparation in the clinic, the clinician will generally pre-warm all materials needed to 30-37° C. Sample viability was tested after thawing the sample and warming it up to approx. 30° C. by two alternative methods: (i) thawing from −80° C. to 4° C., then to room temperature (RT) and 30° C., or (ii) the same procedure but without the 4° C. step.

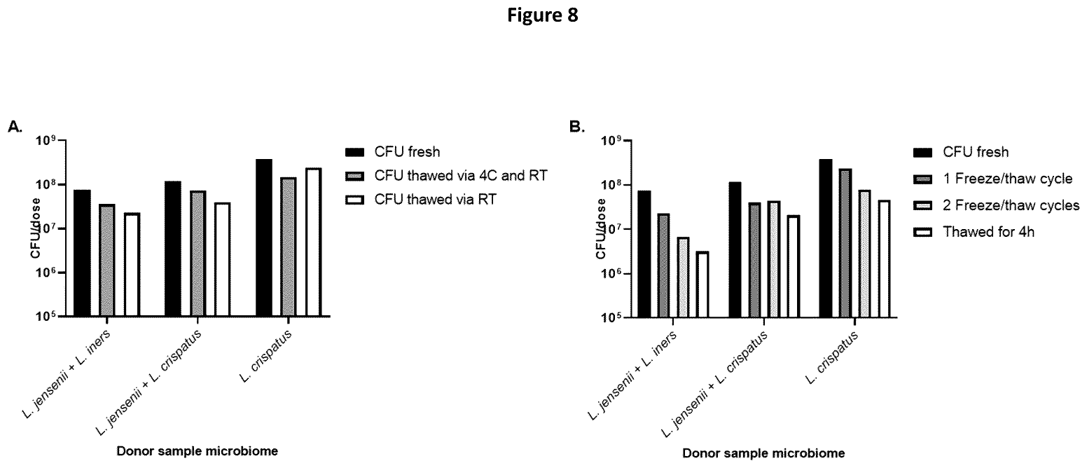

FIG. 8A shows the results, wherein ‘CFU fresh’ is the CFU as obtained when plating the fresh sample directly after sampling and prior to any storage. ‘CFU thawed via 4 C and RT’ is the CFU after taking the sample out of −80° C. storage and gradually warming it up by placing it at 4° C. for 30 min, then at room temperature (RT) for 30 min, and then at 30° C. ‘CFU thawed via RT’ follows the same procedure, but skips the step at 4° C.

CFU and cell viability remained stable in all tested samples, wherein the former method resulted in slightly better viability for 2 samples, while for a third one the latter method was slightly better (FIG. 8A). However, differences were small and generally acceptable viability was maintained. This suggests that the 4° C. step can be omitted, and method (ii) was used for implementation in the clinic during vaginal administration of the preparations.

Repeated Freeze-Thaw Cycles

Another aspect of the stability is the capacity to withstand repeated freeze-thaw cycles. This is practically relevant in cases where recipients fail to report for an appointment to receive the substantially complete vaginal microbiota preparation, and the samples might have to be re-frozen for administration at a later time point. Alternatively, shipments might be delayed and need refreezing after thawing en route. Hence, the effect of freeze-thaw cycles on the samples, as well as a prolonged incubation at room temperature (RT) were assessed.

FIG. 8B shows preliminary data, wherein ‘CFU fresh’ is the CFU as obtained when plating the fresh sample directly after sampling and prior to any storage. ‘1 Freeze/thaw cycle’ is the CFU after taking the sample out of −80° C. storage and leaving it at RT for about 40 min and pre-warming it to around 30-37° C. during about 30 min. ‘2 Freeze/thaw cycles’ is the CFU after repeating this freeze-thaw cycle one more time but without heating to 30-37° C., with the sample being stored at −80° C. in-between thaw cycles for 2-3 weeks. ‘Thawed for 4 h’ is the CFU after taking the sample out of −80° C. storage and leaving it at room temperature (RT) for 4 hours instead of 30 min prior to plating. The results indicate that undergoing the freeze-thaw cycle twice reduces viability more than undergoing the cycle just once. L. crispatus-L. jensenii mixtures were most robust and could withstand repeated freeze-thaw cycles better than other tested compositions (FIG. 8B).

A prolonged (4 h) incubation at RT reduced viability by more than 1 log or close to 1 log and hence should be avoided. These preliminary data demonstrate that all samples could maintain cell viability for at least one freeze-thaw cycle, when not left for longer periods at RT. This is useful in a clinical setting, where samples can be refrozen and do not need to be discarded.

### Example 6: Matching Donor and Recipient for Vaginal Administration of the Preparation

To evaluate if a donor sample could be selected in vitro prior to administration, the following assay was performed. CVS material from recipient 2, who was B. vaginale-dominant (previously called Gardnerella vaginalis) (FIG. 8B), was processed according to the biobanking protocol (see Example 8), plated on Gardnerella vaginalis medium (ThermoFisher Scientific, PB5067A) and grown overnight at 37° C. in an anaerobic jar with gas pack. After adding 1 mL sterile saline to the plate, colonies were scraped off, resuspended, and diluted to a density of about 0.5 McFarland standard. 100 μL of the diluted recipient sample was plated on four new Gardnerella vaginalis plates and air-dried for 5 min. Then, four sterile holes with a diameter of approx. 3 mm each were punched into each plate. Donor-derived substantially complete vaginal microbiota preparations were deposited into each hole (2 replicates with undiluted and 2 replicates with 5× diluted sample). These plates were incubated anaerobically at 37° C. in an anaerobic jar with gas pack. The presence and size of halos formed around the holes were measured after 5 and 7 days. A halo is a zone around the well that is characterized by a distinct change of appearance that can be determined visually. The absence or inhibition of proliferation of the pathogenic cells from the recipient could be observed around the holes, if the hole contained agents that negatively affected the proliferation of the pathogenic cells (bacterial exclusion zone). The larger the halo around the well, the stronger the inhibition/exclusion. Since the entire substantially complete vaginal microbiota preparation was administered, the effect observed here is the result of the complete composition. The halo assay thus provides a readout to assess which donor would be suitable and would have the best chance to inhibit cell proliferation of the pathogens, e.g., those residing in the vaginal cavity of a recipient. The ability of a lactobacilli-dominated microbial preparation to inhibit resident pathogen growth in a niche (e.g., the vaginal cavity of a recipient) is expected to aide engraftment and stably colonization upon administration to the niche. The Gardnerella vaginalis medium contains no antibiotics for which lactobacilli are sensitive and hence it is a representation of the administration without antibiotics.

The results are summarized in Table 4.

TABLE 4

| | | | | | | | | | | Halo |
|---|---|---|---|---|---|---|---|---|---|---|
| | | | | | | | | | Halo | (mm) |
| | L. | L. | L. | L. | Other | Pathogens | | Viable | (mm) | 5x |
| Donor | crispatus % | gasseri % | iners % | jensenii % | lactobacilli % | total % | pH | cells/dose | undiluted | diluted |
| 1 | 88.7 | <0.05 | 4 | 7.2 | <0.05 | 0 | 3.8 | 1.3E09 | 3 | 1.5-2 |
| 5 | 97.6 | 0 | 0.3 | 2 | 0.1 | 0 | 4.0 | 3.4E08 | 3 | 0 |
| 4 | 97.3 | 2.2 | 0 | 0.1 | 0.2 | 0 | 3.9 | 4.1E08 | 0 | 0 |
| 9 | 0 | 0 | 99.6 | 0 | 0 | 0.1 | 4.1 | 3.1E08 | 0 | 0 |

The undiluted samples from donors 1 and 5 showed equally large halos (3 mm) indicative of a strong inhibitory effect on the growth of the pathogens. The same samples were diluted 5-fold, which resulted in reduced efficacy, which can be seen by the reduction of the halo for donor 1. For donor 5, the dilution abolished the inhibitory effect. The samples from donors 4 and 9 were not effective in inhibiting pathogen growth at either concentration.

These data suggest that donor 1 could be a potentially better match for administration of the substantially complete vaginal microbiota preparation for this recipient than donors 5, 4 and 9, allowing for an increased chance of success to treat the dysboitic microbiome residing in this recipient. This sample of donor 1 was hence used for administration to recipient 2 in a clinical setting (described below in Example 7).

### Example 7: Treating Dysbiotic Microbiomes Through Vaginal Administration of the Preparation

This Example demonstrates how a substantially complete vaginal microbiota preparation can be used to revert a recipient's dysbiotic microbiome to a donor's healthy microbiome using vaginal administration, thereby treating the dysbiotic microbiome.

Administration of Substantially Complete Vaginal Microbiota Preparation and Results

The substantially complete vaginal microbiota preparation sample was first thawed by moving it from −80° C. to room temperature for 40 minutes. Thereafter, the sample was placed in an incubator at 37° C. for 30 minutes. The tube containing the sample was then gently inverted 5 times for mixing. The sample was drawn up into a 5 mL syringe and applied intravaginally to the recipient lying in the lithotomy position using an insemination catheter attached to the 5 mL syringe. The recipient was instructed to remain lying down in a horizontal position for 30 minutes following the sample being inserted intravaginally.

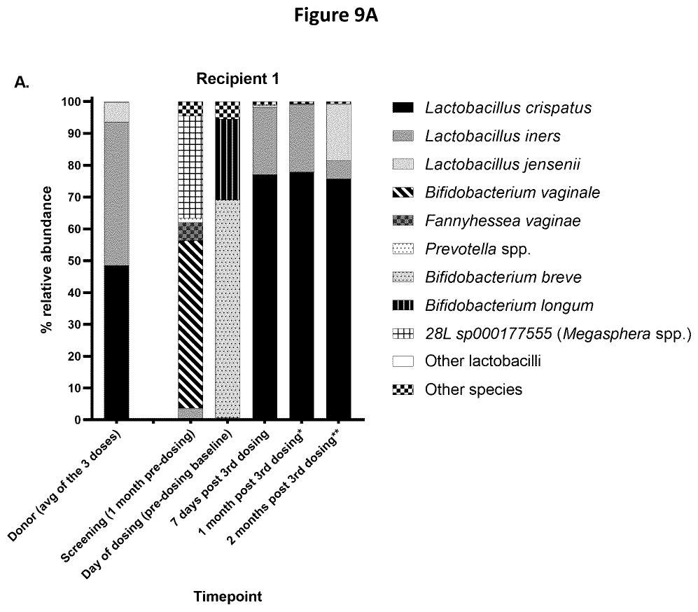

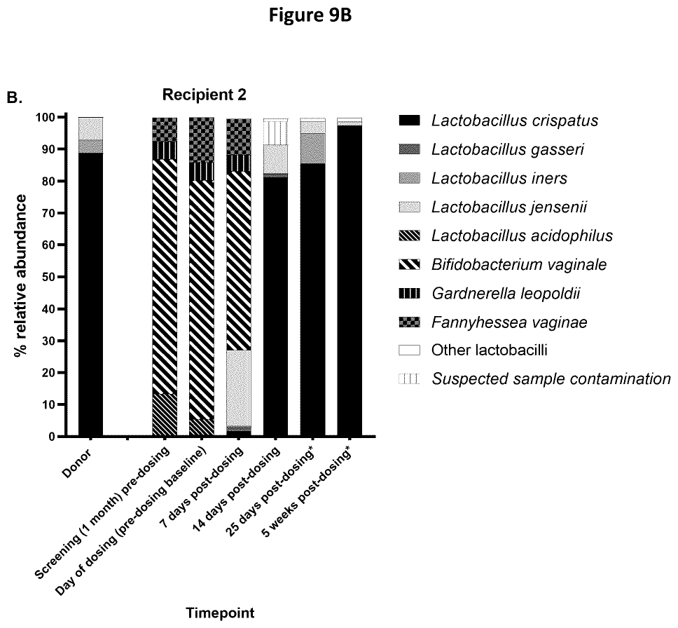

Two representative changes of a dysbiotic vaginal microbiota after administration of a substantially complete vaginal microbiota preparation are shown in FIG. 9. Recipient 1 received 3 doses (1 dose/day over the course of 3 consecutive days) (FIG. 9A), while recipient 2 received only a single dose (FIG. 9 B). To increase the chance of success for recipient 2, an in vitro donor-recipient matching assay was performed as described in Example 6. The sample that performed best in that assay, from donor 1 (see, Table 4), was successfully used for administration in recipient 2. The recipients were not pre-treated with antibiotics prior to or during the administration of the preparation. After administration of the preparation, the microbiome changes of the recipients demonstrate that the substantially complete vaginal microbiota preparation is capable of changing a dysbiotic microbiome to a microbiome more closely resembling that of the donor, by administration of the substantially complete vaginal microbiota preparation alone (i.e, without treatment with antibiotics).

All recipients were enrolled based on their microbiome at screening, which was generally one month before their visit. At their visit for administration of the substantially complete vaginal microbiota preparation, their microbiome was checked again as baseline measurement. Recipient 1 was vaginally asymptomatic, while recipient 2 showed vaginal disease symptoms at the time of administration. Recipient 2 reported a marked decrease in symptoms 1 week after administration of the preparation, and full disappearance of all vaginal disease symptoms after 2 weeks.

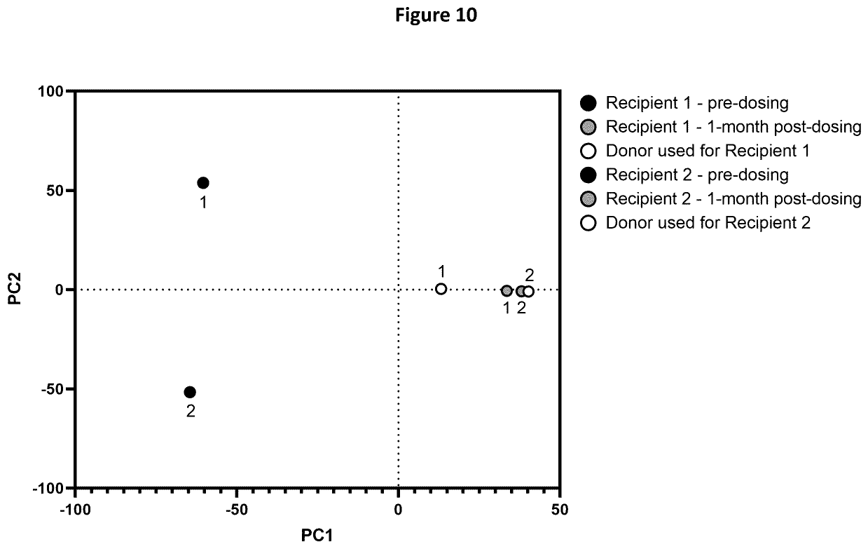

The post-administration microbiome of the recipients appeared similar to the donors', in what could be termed ‘microbiome twinning’ and remained fairly stable. Overall, the post-administration recipients clustered together in an unsupervised principal component analysis (PCA) plot with the donor samples, while the pre-administration recipients form a separate cluster (FIG. 10). This suggests that the vaginal microbiome of the recipient after administration of the preparation did not only change from a dysbiotic state but resembles the vaginal microbiome composition of the healthy, non-dysbiotic donor.

The microbiome compositions of the recipients pre- and post-administration of the substantially complete vaginal microbiota preparation(s), and the composition of the respective donor are summarized in Table 5. The Table shows the relative abundances (determined by Shotgun sequencing) of species in donor and recipient samples, wherein after administration of the preparation ‘microbiome twining’ was observed in each case, i.e. donor species cultivated in the vaginal niche of recipients including species that were present in the donor microbiome in low quantities, and were absent in the recipient prior to administration of the preparation. The relevant species where this phenomenon is observed are highlighted in the grey columns.

The post-administration microbiome of the recipient included species that were present in minor quantities in the donor microbiome (Table 5; wherein L. is Lactobacillus and B. is Bifidobacterium), suggesting that the entire stable ecosystem was transferred from the donor to the recipient. It was also observed that these species were stably present for all donation visits of most donors (see, Example 5). The transfer of the entire ecosystem to the recipient through administration of a substantially complete vaginal microbiota preparation may allow for successful engraftement and colonization of the lactobacillus-dominated donor microbiome, even in the absence of an antibiotic pre-treatment used to eradicate residing pathogens and pathobionts and create a substantially empty microbial niche. This might explain why single species or single strain preparations that have previously been tested generally do not work well. Alterntively or in addition, there might be a need for certain substances in the substantially complete vaginal microbiota preparation (e.g., mucin, lactic acid, etc.) to support initial engraftment.

Moreover, while from 2 weeks post-administration onwards the microbiome of recipient 2 was nearly identical to the donor's, with a mixed species composition, one week post-administration L. jensenii was the first dominant Lactobacillus species. This may indicate the existence of ‘pioneer species’, which pave the way for colonization of the other species. This phenomenon might be explained by different substrate utilization capacities or pH optima of different species. However, in some of the other recipients, such as recipient 1, after 1 week the microbiome was already nearly identical to that of the donor. The fluctuation of species within the microbiome observed in the donor was also observed in the recipient, suggesting the transfer of an ecosystem. The material used in recipient 1 was from the same donor but different visits as that used for recipient 2. Across those samples, the species are the same but their relative abundance is different, indicating that the abundance of a species is not necessarily the determining factor. Moreover, the substantially complete vaginal microbiome preparations were effective in both a single and a multiple dose.

Engraftment appears successful when the entire ecosystem is present in the donor material, thus requiring a substantially complete preparation.

Other researchers have previously suggested a harmful role of L. iners. However, L. iners is present in the donor microbiomes that were tested. In women that pass the stringent screening criteria described herein, L. iners may also play a be beneficial role in representing healthy, stable microbiomes. This confirms the selection process to use all four main lactobacillus species for administration of the preparation.

As a blinded approach, to check engraftment by the specific donor microbiome in one of the recipients vaginal niche, the post-treatment recipient microbiome was compared to a library of all donor microbiomes (metagenomic sequencing data). It was possible to identify correct donor sample from the donor library based on the closeness to the recipient microbiome, suggesting that engraftment of the donor microbiome in the recipient was successful.

This Example thus demonstrates the successful treatment of a dysbiotic vaginal microbiome by administration of a substantially complete vaginal microbiota preparation to a dysbiotic recipient, wherein post-administration the recipient's microbiota composition closely resembled the donor's microbiota composition. While antibiotic treatment prior to administration was thought to be required—as proposed by others—these data demonstrate that pretreatment with antibiotics is not required if substantially complete vaginal microbiota preparations are provided that contain a substantially complete ecosystem.

TABLE 5
Relative abundance (%) of vaginal Lactobacillus species and selected vaginal pathogens
after administration of the substantially complete vaginal microbiota preparation.

| | | | | | | Limosilacto- | | |
|---|---|---|---|---|---|---|---|---|
| | L. | L. | L. | L. | L | bacillus | Prevotella | B. |
| Recipient 1 | crispatus | gasseri | iners | jensenii | helveticus | vaginalis | spp. | vaginale |
| Dose 1 donor sample | 42.84 | 0.00 | 47.17 | 9.84 | 0.01 | 0.02 | 0.00 | 0.00 |
| Dose 2 donor sample | 46.65 | 0.00 | 48.30 | 4.89 | 0.00 | 0.05 | 0.00 | 0.00 |
| Dose 3 donor sample | 56.07 | 0.00 | 39.78 | 3.83 | 0.00 | 0.10 | 0.00 | 0.00 |
| Screening | 0.02 | 0.00 | 3.63 | 0.00 | 0.00 | 0.00 | 1.29 | 52.64 |
| Day of (pre-) dosing | 0.08 | 0.00 | 0.02 | 0.00 | 0.00 | 0.00 | 0.55 | 0.04 |
| 7 days post 3rd dosing | 77.05 | 0.00 | 21.17 | 0.50 | 0.02 | 0.02 | 0.31 | 0.00 |
| 1 cycle post 3rd dosing | 77.85 | 0.00 | 21.28 | 0.08 | 0.02 | 0.12 | 0.01 | 0.03 |

| | Fannyhessea | | | 28L sp000177555 | Other | Other |
|---|---|---|---|---|---|---|
| Recipient 1 | vaginae | B. breve | B. longum | (Megasphera spp.) | lactobacilli | species |
| Dose 1 donor sample | 0.00 | 0.00 | 0.00 | 0.00 | 0.11 | 0.04 |
| Dose 2 donor sample | 0.00 | 0.00 | 0.00 | 0.00 | 0.09 | 0.04 |
| Dose 3 donor sample | 0.00 | 0.00 | 0.00 | 0.00 | 0.10 | 0.05 |
| Screening | 5.75 | 0.00 | 0.00 | 32.23 | 0.00 | 4.44 |
| Day of (pre-) dosing | 0.00 | 68.49 | 25.31 | 0.00 | 0.00 | 5.53 |
| 7 days post 3rd dosing | 0.00 | 0.00 | 0.00 | 0.00 | 0.04 | 0.92 |
| 1 cycle post 3rd dosing | 0.00 | 0.00 | 0.00 | 0.00 | 0.16 | 0.57 |

| | | | | | | Limosilacto- | |
|---|---|---|---|---|---|---|---|
| | L. | L. | L. | L. | L. | bacillus | L. acido- |
| Recipient 2 | crispatus | gasseri | iners | jensenii | helveticus | vaginalis | philus |
| Donor sample (1 dose) | 88.71 | 0.05 | 4.03 | 7.16 | 0.02 | 0.01 | 0.00 |
| 26 days pre-dosing | 0.03 | 0.00 | 0.03 | 0.00 | 0.00 | 0.00 | 13.30 |
| Day of (pre-) dosing | 0.36 | 0.00 | 0.24 | 0.00 | 0.00 | 0.00 | 4.85 |
| 7 days post-dosing | 1.70 | 1.14 | 0.45 | 23.80 | 0.08 | 0.03 | 0.00 |
| 14 days post-dosing | 81.20 | 1.10 | 0.13 | 8.90 | 0.80 | 0.00 | 0.02 |
| 25 days post-dosing* | 85.30 | 0.24 | 9.40 | 3.76 | 0.77 | 0.07 | 0.02 |
| 5 weeks post-dosing* | 97.30 | 0.05 | 0.03 | 1.10 | 0.94 | 0.00 | 0.02 |

| | B. | Fannyhessea | Gardnerella | Other | Suspected sample |
|---|---|---|---|---|---|
| Recipient 2 | vaginale | vaginae | leopoldii | lactobacilli | contamination |
| Donor sample (1 dose) | 0.00 | 0.00 | 0.00 | 0.02 | 0.00 |
| | | | | 0.00 | |
| 26 days pre-dosing | 73.50 | 7.50 | 5.50 | 0.00 | |
| Day of (pre-) dosing | 74.80 | 14.10 | 5.60 | 0.00 | |
| 7 days post-dosing | 56.00 | 11.20 | 5.10 | 0.00 | |
| 14 days post-dosing | 0.01 | 0.00 | 0.00 | 0.17 | 7.24 |
| 25 days post-dosing* | 0.03 | 0.00 | 0.00 | 0.09 | |
| 5 weeks post-dosing* | 0.01 | 0.00 | 0.00 | 1.23 | 0.12 |

### Example 8: Elevated Vaginal Inflammatory Markers in Dysbiotic Women

This Example demonstrates that dysbiotic vaginal microbiomes represent a local inflammatory state which is distinct from healthy women.

Biobanking of Screening Cohort CVS for Biomarker Analysis

CVS samples were collected as described for the for the substantially complete vaginal microbiota preparation (see, Example 2) but with the following differences: Samples were immediately placed on ice when received and kept cold throughout the entire processing procedure. Cold instead of room temperature sterile saline was used, and samples were frozen to −80° C. without the use of a CoolCell. Instead of adding 1 ml sterile saline per sample, 2.5 μL was added per mg sample so all samples were normalized for weight prior to analysis. The quality checks were omitted (CFU, pH, sperm cell check), except sequencing.

Inflammatory Protein Analysis

Bacterial vaginosis (BV)-associated and dysbiotic vaginal microbiota have been linked to increased concentrations of several proinflammatory cytokines such as IL-1a, IL-1b, IL-8, IL-12, IL-18, and FMS-related tyrosine kinase 3 ligand (FLT-3L), as well as matrix metalloproteinases (MMPs). To investigate the inflammatory state of the asymptomatic and dysbiotic, and healthy women in the screening cohorts, 0-link analysis (BioXpedia, Aarhus, DK) was performed. 0-link is a highly specific, targeted proteomics method, in which qPCR is performed on proteins by using antibody-linked oligonucleotides. Different panels of multi-well plates are available that target different subsets, such as the inflammation panel used here to quantify 92 inflammatory markers.

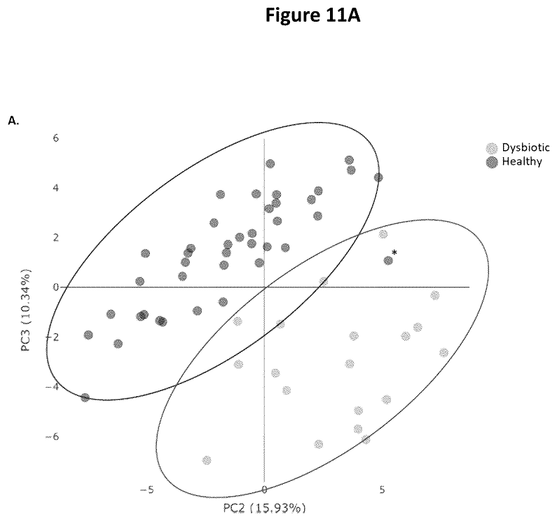

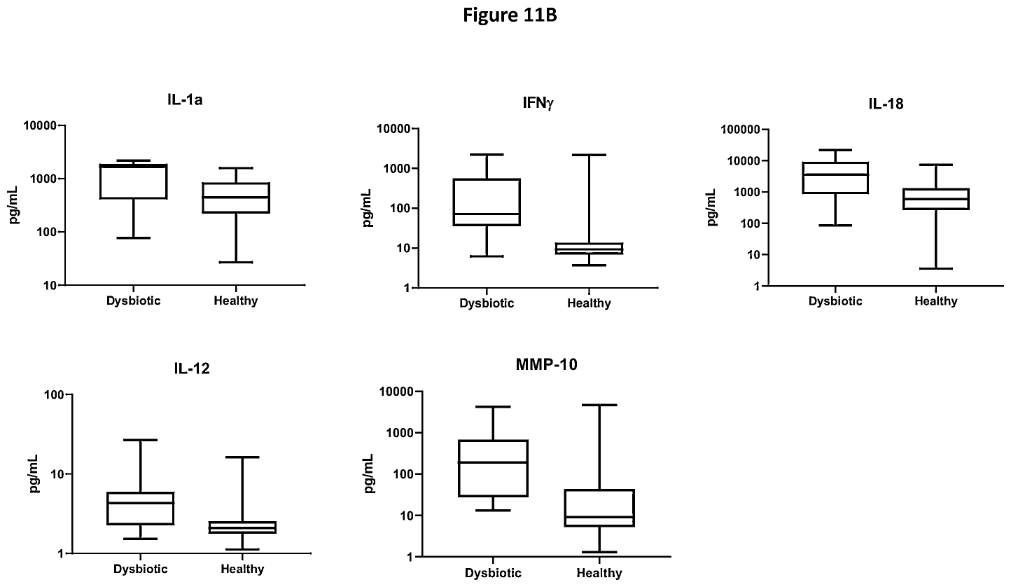

A clear separation based on inflammatory protein markers was observed between the dysbiotic and healthy groups in the screening cohort (FIG. 11A). Only one healthy microbiome sample clustered with the dysbiotic samples, which could be due to an undetermined infection that is not picked up by bacterial metagenome sequencing, such as, e.g., Candida (this data was generated pre-gynecological examination and pathogen screen, and all women were asymptomatic). Several proinflammatory cytokines, known to be upregulated in BV, were also upregulated in the dysbiotic participants, such as IFNγ, IL-1α, IL-12, IL-18, and MMP-10 (see, References and FIG. 11 B), which belong to Th1 cytokines (IFNγ, IL-12), innate/inflammasome-associated cytokines (IL-1, IL-18), and metalloproteinases (e.g., MMP-10). Horizontal lines represent medians.

### Example 9: Administration of the Substantially Complete Vaginal Microbiota Preparation Reduces the Inflammatory State of the Vaginal Niche

This Example describes the change of the dysbiotic vaginal microbiome induced by administration of a substantially complete vaginal microbiota preparation by reducing of the local inflammatory state.

Biobanking of Donor and Recipient CVS for Biomarker Analysis

For several points in time, CVS samples taken from recipient 2 were used for Olink inflammatory biomarker analysis. Samples were processed in the same way as the biobanking samples of the screening cohort in Example 8. For each donor, including the one used for recipient 2 the 5^{th} visit was not used for administration but instead processed according to the biobanking method. This yielded a number of aliquots for the sole purpose of biobanking.

Inflammatory Protein Analysis

To be able to compare the samples from recipient 2 with dysbiotic and healthy vaginal microbiome samples without having batch-to-batch variation between Olink plates, a subset of samples from the screening (based on sample availability) was run again on the same Olink inflammation plate as the recipient samples. The biobanking sample of the donor used for recipient 2 was run on the same plate.

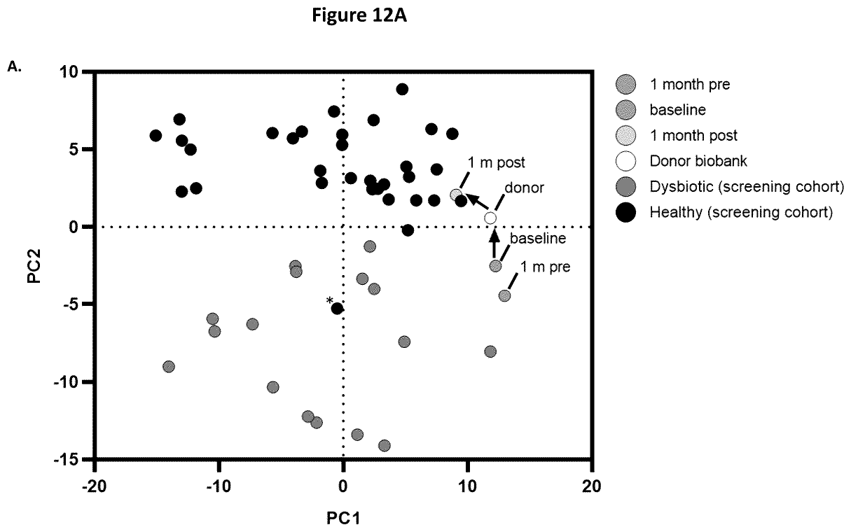

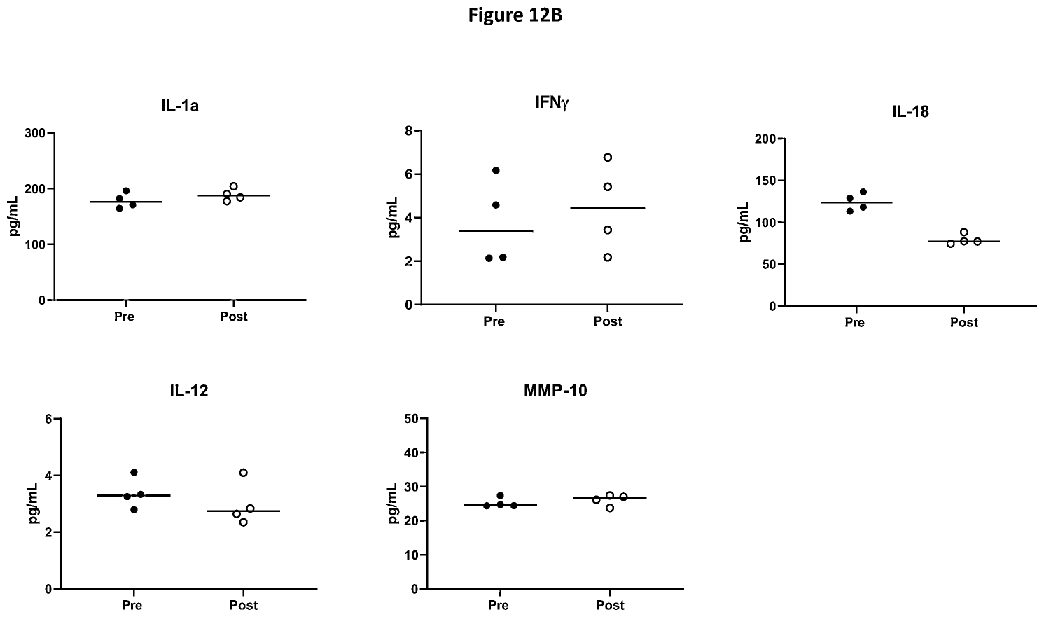

The subset of re-run screening cohort samples again showed a clear separation based on inflammatory protein markers between the dysbiotic and healthy groups (FIG. 12A). The donor biobanking sample clustered together with the healthy/lactobacilli-dominant screening samples. Pre-administration, i.e., prior to administration of the substantially complete vaginal microbiota preparation to the recipient, the samples of recipient 2 clustered with the dysbiotic samples and were separated from the donor as well as healthy cohort (FIG. 12A). One month after administration of the preparation to the recipient, the recipient's microbiome clustered with the healthy cohort, which can be seen by the “1 m post” data point in the PCA plot of FIG. 12A, which is in close proximity to the solid circles representing the healthy subjects. The administration of the substantially complete vaginal microbiota preparation thus shifted the recipient's vaginal microbiome from a dysbiotic microbiome towards the microbiome of the healthy donor (FIG. 12 A). The post-administration and donor dots are not exactly overlapping, which might be due to slight differences in the donor samples that were assayed for Olink and used for administration to the recipient (same donor but different days of donation and preparation). Further, since healthy donor microbiomes also fluctuate in their relative amounts of lactobacilli (see FIGS. 6A-C), slight variations between donor and recipient are to be expected.

Though limited by the small number of available data points, the selected BV-related proinflammatory markers that were differentially expressed in dysbiotic and healthy cohorts (FIG. 11 B) were either not changed or showed a trend of being downregulated post-administration (FIG. 12 B), suggesting that the treatment of dysbiosis by administration of the substantially complete vaginal microbiota preparation may treat or ameliorate local inflammation in the vaginal cavity.

### Example 10: Clinical Studies

This Example describes two clinical studies aimed at evaluating the efficacy of using a substantially complete vaginal microbiota preparation as provided herein as a treatment of vaginal dysbiosis.

Two clinical studies are performed, which explore and map the shifts in vaginal microbiomes as a consequence of treatment with a substantially complete vaginal microbiota preparation.

The main objective of the first study is to evaluate the effect of 3 sequential substantially complete vaginal microbiota preparation doses from healthy donors to healthy, asymptomatic volunteer women screened to have vaginal dysbiosis based on criteria defined from metagenomic sequencing of a vaginal swab sample. Administration of the substantially complete vaginal microbiota preparation will be performed 3 times at 3 different visits on 3 consecutive days and subjects and their vaginal microbiomes are followed until approximately 6 months after the first dose. The study is a single-site, randomised, double-blind, placebo-controlled, two-armed, parallel-group study in n=40 healthy volunteer women aged 18-45 years. It involves 11 study visits in total (FIG. 14).

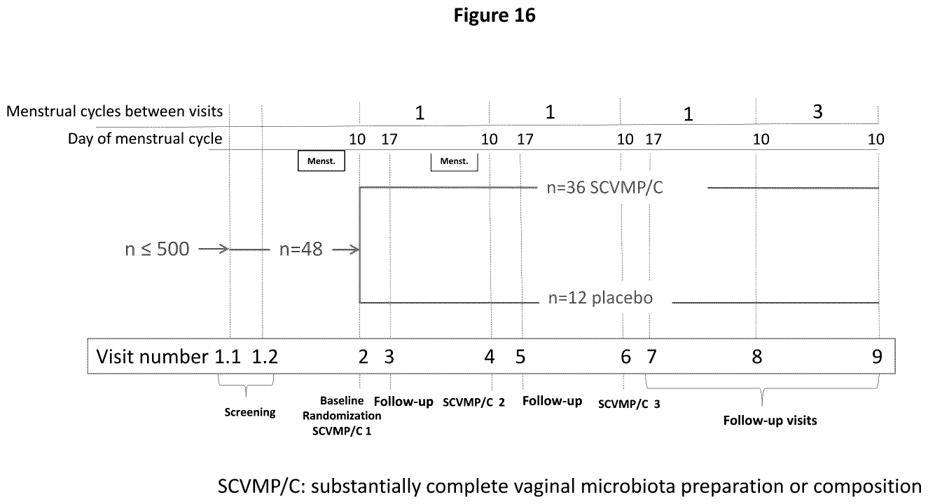

The main objective of the second study is to evaluate the effect of up to 3 individual doses separated by follow-up visits from healthy donors to either healthy, asymptomatic or symptomatic women volunteers, screened to have vaginal dysbiosis based on criteria defined from metagenomic sequencing of a vaginal swab sample. The treatment with a single dose is performed up to 3 times, separated by follow-up visits and subjects and their vaginal microbiomes will be followed out until approximately 6 months after the first dose. Each dose is evaluated individually for successful shifting of the recipient's dysbiotic microbiome. The second dose is only administered if it is determined that the first dosing did not successfully shift the vaginal microbiome towards a healthy, lactobacillus-dominant taxonomic profile in the follow-up visit after the first dosage. Similarly, the third dosing is performed only if the follow-up visit after the second dosing showed that a dysbiotic vaginal microbiome remained. This study is a single-site, randomised, double-blind, placebo-controlled, two-armed, parallel-group study in n=48 healthy volunteer women aged 18-40 years. It involves 10 study visits in total (FIG. 16).

The investigational product is a cryovial containing approx. 1.4-1.8 mL of the substantially complete vaginal microbiota preparation. The donors used for obtaining the substantially complete vaginal microbiota preparation underwent the screening and follow-up procedure described herein. The reference product (placebo) is provided in similar cryovials as the test product but only contains approx. 1.3 ml of sterile saline solution.

The trials include a screening visit where all criteria for participation are checked and a vaginal swab is obtained to check if pre-defined dysbiosis criteria are met based on metagenomic sequencing (see below). If, and when, all criteria are confirmed, a baseline visit, visit 2, is scheduled to be performed around day 8 (Study 1) or 10 (Study 2) of the following menstrual cycle of each individual subject. Besides baseline assessments, the subject are randomised to “active” substantially complete vaginal microbiota preparation or “placebo” arms in a ratio of 3 (active): 1 (placebo) and the first dose is given. For Study 1, Visits 2, 3 and 4 (performed on days 8, 9 and 10 in the same menstrual cycle as Visit 2) consist of receiving 1 dose each for a total of 3 doses. For Study 2, the second dose is given at day 10 in the participant's cycle in case dosing 1 did not work. Dosing 3 is performed at visit 6 also on day 10 of the participant's cycle in case dosing 2 did not work.

In total, for Study 1, 7 follow-up visits (visit 5-11) are scheduled to take place during a period of up to 6 months after the last dose. For Study 2, in total 3 follow-up visits (visit 7-9) are scheduled to take place during a period of up to 6 months after the first dose. All follow-up visits are scheduled to occur around day 8 (Study 1) or 10 (Study 2) in the subject's menstrual cycle with one menstrual cycle between visits. At all follow up visits, a metagenomic sequencing assessment of the microbiota in a vaginal sample is performed.

To be eligible for inclusion, the participant must fulfil all of the following criteria:
able and willing to give written informed consent.
between ≥18 to ≤45 (Study 1) or ≥18 to ≤40 (Study 2).
generally healthy, as determined by the investigator.
free of any vaginal symptoms (Study 1 only).
pre-menopausal.
meets the following pre-defined criteria of vaginal dysbiosis: Combined Lactobacilli relative abundance below 10% and combined relative abundance of Gardnerella+Atopobium+Prevotella above 20% based on metagenomic sequencing of vaginal sample.
has regular, predictable menstrual cycles of known length or has been amenorrhoeic for at least 3 months due to use of a long-acting progestin or hormonal contraceptives.
willing to be asked questions about personal medical, sexual, and behavioural history.
willing to undergo three or up to three treatments with the SCVMP.
willing to self-collect cervicovaginal secretions and vaginal swab samples at a clinic.
willing to abstain from vaginal intercourse, unless using condoms, without the use of adjunctive spermicide or lubricant for at least one menstrual cycle length (28 days) after the treatments.
willing to avoid taking baths, swimming, sitting in a hot tub, or wearing thong underwear for at least one menstrual cycle length (28 days) after the treatment.
willing to abstain from using insertive vaginal feminine products (e.g., tampons, menstrual cups, sex toys), vaginal cleansing products, spermicides, lubricants, or other vaginal products not approved by the study investigators for at least 1 menstrual cycle length (28 days) after a treatment.
a wish to not become pregnant in the study participation period and willing to use condoms during sexual intercourse in the study participation period (Study 2 only).

The presence of any of the following criteria excludes the participant from the study:

Participants who are post-menopausal, defined as more than 12 consecutive months of amenorrhea without another known cause than use of long-acting progestin or hormonal contraceptives.

Participants who are pregnant, breastfeeding and/or have been pregnant within the last two months, or in the case of Study 2 who plan/wish to become pregnant in the coming 6 months.

For Study 1, for participants currently of childbearing potential, but not using an effective method of contraception:
a. Complete abstinence from intercourse two weeks prior to the treatment, throughout the clinical trial, until the completion of follow-up procedures or for two weeks following discontinuation of the study participation in cases where participant discontinues the study prematurely. (Participants utilising this method must agree to use an alternate method of contraception if they should become sexually active and will be queried on whether they have been abstinent in the preceding 2 weeks when they present to the clinic for the Final Visit).
b. Has a male sexual partner who is surgically sterilised prior to the Screening Visit and is the only male sexual partner for that participant.
c. Use of acceptable method of contraception, such as a spermicide, mechanical barrier (e.g., male condom, female diaphragm), or contraceptive pill. The participant must be using this method for at least 1 week following the end of the study.
d. Use of any non-hormonal intrauterine device (IUD) or contraceptive implant with published data showing that the highest expected failure rate is less than 1% per year. The participant must have the device inserted at least 3 months prior to the first Screening Visit, throughout the study, and 2 weeks following the end of the study.

Women in same-sex relationships, or who are likely to engage in sexual relationships with other females throughout the study (Study 1 only).

Participants who have HIV/AIDS or other immunodeficiency.

Participants who test positive at screening for HIV, chlamydia, gonorrhoea, Mycoplasma genitalium, and/or Trichomonas vaginalis; and for Study 2 also and/or urine hCG.

Participants with a current vaginal Candida infection requiring treatment that by the opinion of the investigator contraindicates participation in the current trial (Study 1 only).

Participants may not be receiving treatment involving experimental drugs. If the participant has been in a recent experimental trial, these must have been completed not less than 30 days prior to this study.

Participants who have undergone some sort of procedure involving trauma to the cervix within the last 2 months prior to screening (i.e., IUD removal, cervical cryotherapy, or cervical laser treatment.

Participants with any known condition requiring regular use of antibiotics, that would suggest the participant is likely to require antibiotic treatment during the study.

Systemic and/or vaginally applied antibiotic use within the last month prior to screening (Study 1 only).

Participants with any social, medical, or psychiatric condition that in the opinion of the investigator would make it unlikely for the participant to comply with the study or would complicate interpretation of data from her participation.

Participants with a history of drug or alcohol abuse that in the opinion of the investigator would make it unlikely for the participant to comply with the study or would complicate interpretation of data from her participation (Study 1 only).

Participants with a history of gynaecological cancers, gynaecological conditions, or surgical gynaecological medical history, which, in the opinion of the investigator, precludes participation (Study 1 only).

Participants with abnormal finding on screening exam, which, in the opinion of the investigator, precludes participation.

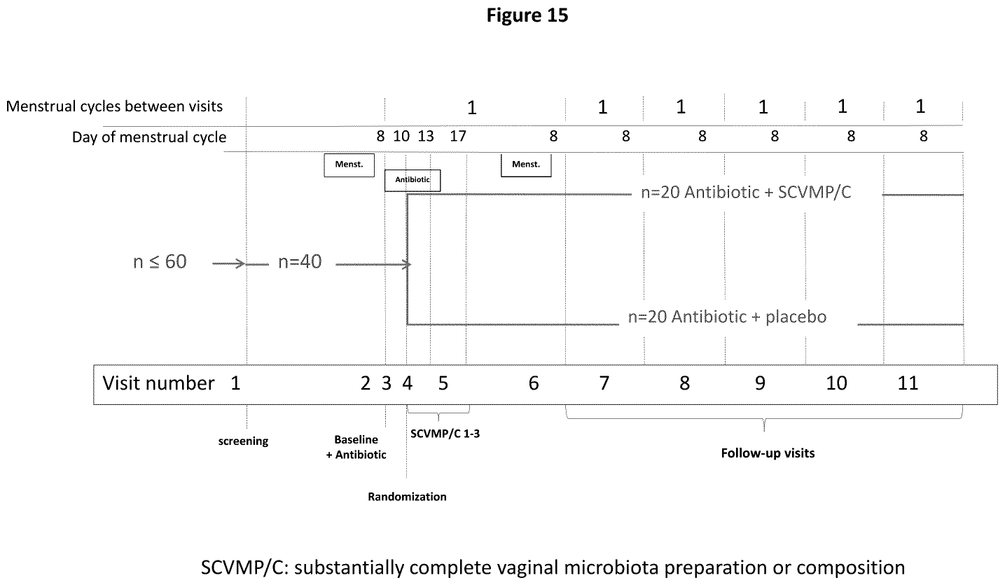

A modified clinical trial that is identical to Study 1, but further administers an antibiotic agent in addition to the substantially complete vaginal microbiota preparation is shown in FIG. 15.

Preliminary Results

Across the 2 studies, the following success rates were observed in shifting the vaginal microbiomes from dysbiosis to a Lactobacillus-dominant healthy vaginal microbiomes that had similar or near similar taxonomic profiles as the donor sample used for the administration of the substantially complete vaginal microbiota preparations. Data is stratified on method of contraception in the recipients and shown only for recipients that were randomized to active intervention (not placebo recipients) (Table 6).

TABLE 6

| | Intrauterine device | Other form of hormonal | Other contraception |
|---|---|---|---|
| | (hormonal and non- | contraception than | method than |
| | hormonal) | intrauterine device | hormonal |
| Total number of recipients | 7 | 8 | 9 |
| dosed with the substantially | | | |
| complete vaginal microbiota | | | |
| preparation | | | |
| Number of recipients with | 0 out of 7 | 3 out of 8 | 3 out of 9 |
| successful shift to healthy | | | |
| vaginal microbiome after | | | |
| dosing | | | |
| Percentage share of | 0% | 38% | 33% |
| recipients dosed that shifted | | | |
| to a healthy vaginal | | | |
| microbiome | | | |

Out of 8 treated dysbiotic subjects using non-IUD hormonal contraception, 3 (38%) successfully shifted from a dysbiotic vaginal microbiome to a healthy vaginal microbiome. Similarly, out of 9 treated dysbiotic subjects that did not use any hormonal contraceptions, 3 (33%) successfully shifted from a dysbiotic vaginal microbiome to a healthy vaginal microbiome. Thus, a total of 6 out of 17 subjects (35%) in these two groups successfully shifted from a dysbiotic vaginal microbiome to a healthy vaginal microbiome. All 7 dysbiotic subjects wearing an intrauterine device (IUD) were not successful in reverting their dysbiotic vaginal microbiome. Among the 7 participants using IUDs, 4 had the hormonal IUD brand Mirena, 1 had hormonal IUD brand Jaydess, 1 had hormonal IUD brand unknown and 1 participant had a copper IUD also with an unknown brand. The data suggest that at least for recipients not using an IUD during treatment, about one third of subjects were successfully treated using a substantially complete vaginal microbiome preparation. This rate of success was achieved even in the absence of (pre)-treatment with antibiotics. This suggests that successful treatment of dysbiosis—by administering the substantially complete vaginal microbiome preparations described herein—is feasible in the absence of antibiotics and the adverse effects associated with their uses.

### Example 11: Multiparameter Assessment of Various Dosage Formulations Using Simulated Vaginal Fluid

A variety of suitable dosage forms were assessed for formulating the substantially complete vaginal microbiota preparation described herein, including formed gels, lyophilized gels, tablets, and films A number of excipients were assessed to achieve suitable dosage forms, including mannitol, micro-crystalline cellulose, mucin (porcine, Sigma), hyaluronic acid (Sigma), maltodextrin, Guar gum (Sigma), inulin (Sigma), alginic acid (sodium alginate, Dupont), polyvinyl alcohol (PVA Parteck SRP 80, Merck), sodium CMC (Ac-Di-sol, sodium carboxymethyl cellulose, DuPont), polyvinylpyrrolidone (Kollidon (PVP), BASF), hydroxypropyl methylcellulose (Methocel K4M (HPMC), Colorcon), poloxamer (poloxamer 407 (Kolliphor), BASF), Carbopol (Carbopol 934, Serva), lactic acid, and acetate buffer.

Basic Formulations

Formed gels were prepared using the excipients including, e.g., hyaluronic acid, sodium alginate, HPMC/PVP, and poloxamer 407. The gels were pH adjusted to maintain a pH about pH 3.4-pH 3.9. A combination of lactic acid and acetate buffers were assessed.

Tablets were prepared using the excipients such as bulking agents including, e.g., microcrystalline cellulose, HPMC/PVP, maltodextran, and poloxamer 407 and compression. Lyophilized excipients were also evaluated to determine if mucoadhesion and/or gelling is improved. A combination of lactic acid and acetate buffer salts was assessed for inclusion. Tablet tensile strength was targeted at about 1.0 MPa, to ensure lack of excessive breakages and good processability. The target for pH of a reconstituted tablet (e.g., inside the vaginal cavity) was about pH 3.4-pH 3.9. The disintegration profile in a low liquid volume environment (e.g., inside the vaginal cavity) was also assessed.

For lyophilized gels, a number of excipients were lyophilized, including, e.g., hyaluronic acid, sodium alginate, HPMC/PVP, and poloxamer 407. All gels were pH adjusted to maintain a pH about pH 3.4-pH 3.9. A combination of lactic acid and acetate buffers were assessed. The target lyophilized appearance was a clean, uniform cake. The target water content was generally less than 3% w/w water (e.g., to increase viability of drug substance). The target reconstitution time in a vial was less than 2 minutes with hand swirling.

For films (air-dried) PVA in various concentrations was assessed. For example, PVA was supplemented with sodium CMC and other excipients to modify drying times, final flexibility of films, mucoadhesion etc. A combination of lactic acid and acetate buffers was assessed, and pH effect on immersion in simulated vaginal fluid evaluated. The target film properties included sufficient flexibility for application (e.g., to the vaginal tract), non-tackiness, acceptable drying times for ease of processing, a suitable film thickness, and disintegration profile in a low liquid volume environment (e.g., inside the vaginal cavity), as well as effective release and engrafting of drug substance. The target for pH of a reconstituted film (e.g., inside the vaginal cavity) was about pH 3.4-pH 3.9.

All components were assessed for optimisation of viscosity (e.g., at 37° C., e.g., inside the vaginal cavity), enhancement of mucoadhesion (e.g., to epithelial, mucosal surfaces e.g., inside the vaginal cavity) and/or positive impact on drug substance stability (e.g., bacterial viability upon formulation and storage). Ideal formulations show little to no flow on suitable vertical surfaces and maintain high bacterial viability (e.g., CFU count) both upon formulation and during (long-term) storage.

Formulation selection parameters that were assessed included: Mucoadhesion (of reconstituted product, e.g., in the vaginal tract); viscosity (of reconstituted product), e.g., final viscosity for gel-based product needs to be syringeable at ambient temperature and preferably congealed at 37° C. (at body temperature, e.g., in the vaginal tract); total sugar content (of reconstituted product), e.g., ideally at or lower than physiological concentration (about 0.5-1.0 mg/mL); volume of reconstituted product, e.g., up to 3 mL; hydration rate/disintegration rate (e.g., of gel/matrix), e.g., sufficient physical integrity to provide desired release rate; pH, e.g., between about pH 3.4-3.9 (e.g., to promote inhibition of competitive vaginal bacteria); water activity/moisture content (e.g., of dried formulations), e.g., between 0.5-3% water (e.g., for longer term dried formulation stability); microbial diversity, e.g., relative abundance of Lactobacillus species, such as, e.g., L. crispatus, L. gasseri, L. jensenii, and L. iners; total dose/potency, e.g., preferably above 1×10⁵ CFU/VCC, above 1×10⁶ CFU/VCC, above 1×10⁷ CFU/VCC, or above 1×10⁸ CFU/VCC per administration (per dose), shelf-life (not reconstituted) at various temperatures, and microbial limits, e.g., absence of microorganisms such as, Pseudomonas aeruginosa, Candida albicans, Staphylococcus aureus, Ph Eur criteria 5.1.4, 2.6.12 & 2.6.13).

Testing was performed using standard assays, including plate count (e.g., MRS agar) or viable cell count (VCC, e.g., using Quantom Tx automated counting system (AM620) with fluorescent stain), e.g., for life bacteria count, dose determination, shelf-life; rheometer, e.g., for mucoadhesion and viscosity, pH meter, Karl Fisher/water activity meter, Ph Eur testing, e.g., for microbial loads.

Gel Formulations

Gel formulations were prepared with the following excipients:
Hyaluronic Acid at 0.5% and at 2%
HPMC+Carbopol+PVP
Poloxamer 20%+Sodium Alginate 2%
PVA 3%+NaCMC 2%
Poloxamer 20%+Guar gum 2%
Guar gum at 0.5% and 2%
Mucin 5%
Inulin 8%

Flow rates (viscosity, mPa×sec/shear rate 1/sec) under gravity at ambient temperature were determined with 0.5 mL on a 56 mm glass slide for Hyaluronic acid 0.5%, HPMC Kollidon 25+Carbopol, Guar gum 0.5%, Simulated mucus and inulin. Values obtained ranged from 300 to 1×10⁷ mPa×sec at shear rate of 0 to 3 to 7,000 mPa×sec at a shear rate of 100 l/sec, depending on formulation. Guar gum (0.5%) and hyaluronic acid (0.5%) did not appear to form gels, yielding low viscosities. Viscosity against temperature from 15 to 45° C. (with fixed shear rate of 10 s−1) was also determined. Poloxamer showed a thermo-reversible gelling behaviour with an increase in viscosity at about 25° C. (from about 10,000 to about 12,000 mPa× sec, while other formulations mostly showed small changes in their viscosities at various levels between about 500 and 12,000 mPa× sec at 15° C. and about 200 to 11,000 at 45° C., depending on formulation.

The next step included and assessment which of the gel bases could be lyophilized and reconstituted to form an acceptable gel. The appearance of a ‘cake’ was also evaluated to indicate homogeneity.

A simulated vaginal fluid (SVF) was produced, with the following composition: 35 mg of NaCl, 14 mg of KOH, 22 mg of calcium hydroxide, 20 mg of lactic acid, 10 mg of acetic acid, 1.6 mg of glycerol, 50 mg of glucose into 10 mL of water, adjusting to pH 4.2 using HCL.

Single vials were used to simulate a single cervico-vaginal secretion donation with SVF, and volumes used were considered as representative of the range of ˜0.5 mL to 1.5 mL expected from a typical donation. After suitable excipients were determined, these excipients were used to form gels by adding 1.5 mL of SVF. The addition of SVF allowed correct pH balance and simulate the addition of a substantially complete vaginal microbial preparation to the excipient base. Samples were hand-shaken within vials to form homogenous gels and then lyophilized. Some excipients produced acceptable appearance post-lyophilisation. PVA, Guar gum and HA, formed acceptable (clear) gels; Kolliphor P 407 (Poloxamer 407) and Methocel K4M formed white, crystalline cakes; maltodextrin and PVP displayed discolorations (e.g., yellow), whereas sodium alginate, mucin, and NaCMC produced non-uniform cakes with discolorations (e.g., brown). Surprisingly, mannitol which typically forms consistently acceptable cakes, was crystalline which is probably due to the salt contents in the SVF.

The resulting gels were reconstituted with 1.5 mL deionised water post lyophilisation. HA, guar gum and PVA reconstituted easily, with reconstitution times of 2:30 min, 2:30 min, and 1:30 min, respectively. HA formed a homogenous gel with a pH of 4.6. Guar gum formed a non-homogenous gel with a pH of 4.5. PVA formed a homogenous liquid with some foaming with a pH of 4.7. PVP, NaCMC, poloxamer, sodium alginate and HPMC formed semi-homogenous gels. All required more than 3 minutes for reconstitution. PVP dissolved slowly with a pH of 4.1. NaCMC formed a non-homogenous viscous liquid with a pH of 4.9. Poloxamer displayed some foaming with a pH of 5.4. HPMC formed a gel with some foaming with a pH of 4.4. Sodium alginate was non-homogenous with a pH of 5.5. Mannitol formed a homogenous liquid with some crystals remaining with a pH of 4.4.

A muco-adhesion test was conducted to study the effect under gravity of the drug product when applied. For this, a mucosal surface (such as would be present in the vaginal cavity) was simulated. 500 mg of mucin was compressed into disc a with a flat 2 cm tooling to approximately 3 tons of pressure. These were then adhered to a substrate, e.g., the bottom of a plastic box. Each disc was then wetted with approximately 200 μL of deionised water and rubbed with a gloved finger until the surface of the mucin became tacky to the touch. About 0.5 mL of sample (or the complete dosage unit, in the case of tablets and PVA films) was then applied in the horizontal position and allowed to settle for 2 minutes. The box was then raised so that samples were in a vertical position, and a visual assessment was taken with respect to adherence properties (muco-adhesion test) of the Guar gum, poloxamer, mannitol, PVP, hyaluronic acid, sodium alginate, CMC, HPMC, and PVA gel samples. Hyaluronic acid, NaCMC, sodium alginate and HPMC showed the best muco-adhesion in this test.

As poloxamer showed thermo-reversable behavior in earlier tests, the muco-adhesion testing was repeated on a pre-heated plastic box and mucin disc at 37° C. It was confirmed that the gel that formed was more viscous than at ambient temperature and retained on the surface of the wetted mucin disc. This indicates that the poloxamer may be at suitable viscosity at body-temperature

Syringeability was also assessed following adjustment of concentrations: 3% (45 mg) carbopol, 4% (60 mg) sodium alginate, 3% (45 mg) HPMC & 1.9% (30 mg) sodium CMC, and 24% (360 mg) poloxamer. These were prepared using an SVF:lactate buffer mix. The inclusion of a lactate buffer in the pH range of ˜3.4-4 may promote engraftment of the lactic acid bacteria in vivo (e.g., in the vaginal tract), e.g., potentially by minimizing the competition of undesirable bacterial taxa resident in the existing microbial niche. Excipient were mixed with 1.5 mL SVF+lactate buffer. Carbopol and poloxamer both formed homogenous gels after hand shaking with some air bubbles which dispersed when settled. Sodium alginate and HPMC+Na-CMC did not form homogenous gels.

A lyophilized gel format as a dosage form for the substantially complete vaginal microbial preparation can, for example, be produced by blending with excipients followed by lyophilization and packaging, e.g., in vials. The vials can then be reconstituted, e.g., in a clinic setting, with water to form a gel in the vial prior to application of the reconstituted drug product, e.g., by using an applicator, such as a syringe, to administer the composition comprising the substantially complete vaginal microbial preparation to the vaginal tract of a subject.

A frozen gel describes a dosage form for the substantially complete vaginal microbial preparation that is blended with gelling excipients (optionally along with a suitable lactate buffer) and the liquid gel form is then frozen (at −80° C.) and stored in either a vial or a pre-filled syringe, see, e.g., FIG. 13.

Tablet Evaluation

In addition to gels, tablet-pessaries were generated as an additional dosage form. The following aspects were considered: choice of excipient suitable for compression to form tablets and for lyophilisation, as well as to provide acceptable level of mucoadhesion, optional inclusion of lactate buffer with a pH target of about pH 3.5-4, optionally with the aim to have a single tablet prepared from individual cervico-vaginal fluid donations. All of the assessed excipients which produced both acceptable gel formation and acceptable lyophilisation (e.g., to form a reasonably free-flowing powder for further processing) were evaluated (alongside additional excipients) for tabletting quality on a manual tablet press, including Carbopol, HPMC, gelatine, sodium alginate, poloxamer, NaCMC, and pectin. Tablets were compressed at about 1 ton a with 5.5 mm tooling to form tablets. Tablets were gently added to wetted mucin discs and muco-adhesion assessed, as described earlier.

All tablets remained attached for over 5 minutes. Tablets were rinsed every minute to assess impact of adhesion. NaCMC began to swell into a gel. Tablets of carbopol, HPMC, alginate, NaCMC showed sufficient tablet integrity. Poloxamer formed a tablet but with relatively low strength. Pectin and gelatine tablets showed low hardness and some brittleness.

Polymeric Films

Film forming/casting is a potential route of preserving viability in live biological materials. To investigate this, the following excipient test samples were generated:
PVA 3%
PVA 20%
PVA 3%, NaCMC 2%
PVA 20%, NaCMC 5%

All samples were mixed as necessary to make a gel/film and transfer into plastic blister pack (size 0) and were stored at ambient conditions for about 48 hours to set.

Both samples containing 3% PVA did not form suitable films Films containing NaCMC displayed brittleness and were difficult to remove from the blister mould. The 20% PVA sample did form a film and could be removed with ease from the blister mould. The film was flexible and resistant to tearing.

To evaluate whether forming a film with the inclusion of substantially complete vaginal microbial preparation is feasible, a ‘sandwich’ approach to film preparation was tried. This was performed in an upturned Karl Fischer lid (with PTFE lining), to produce a mould with a flat disc design. A 12% PVA solution (from the previous 20%) was used to reduce the initial viscosity and allow more consistent sample preparation via pipette. Preparation was performed as follows:

0.25 mL of 12% PVA was pipetted into each mould, followed by 0.5 mL of SVF (to simulate a substantially complete vaginal microbial preparation). Another 0.25 mL of 12% PVA was pipetted on top to complete the ‘sandwich’. The moulds were left to solidify.

After about 48 hours, discs were formed, which were flexible and left no apparent residue on the PTFE lining. This suggests that the PTFE may be a suitable material for moulding, the film was easily recovered from this surface.

Films were gently added to mucin discs and muco-adhesion assessed, as described earlier. The PVA discs showed no movement from the mucin surface, indicating good muco-adhesion. A wash of about 2 mL of SVF was performed after 5 minutes, which did not displace the PVA discs from the mucin surface.

TABLE 7
Formulation summary:

| | | | Lyophilized/ | |
|---|---|---|---|---|
| | | | frozen liquid | Film-form |
| Excipient | Functionality | Tablet | gel | (air-dried) |
| Hyaluronic acid | Structuring polymer | ++ | + | − |
| Sodium alginate | Structuring polymer | − | − | − |
| Maltodextran | Bulking agent, carbon | − | − | − |
| | source | | | |
| Guar gum | Bulking agent, prebiotic/ | + | + | − |
| | viscosity agent | | | |
| NaCMC + HPMC | Polymer combination | + | + | − |
| Polyvinyl alcohol | Structuring polymer | − | − | ++ |
| Sodium CMC | Polymer/mucoadhsive | ++ | ++ | − |
| Polyvinylpyrrolidone | Structuring polymer | ++ | + | − |
| Hydroxypropyl | Structuring polymer | ++ | ++ | − |
| methylcellulose | | | | |
| (HPMC) | | | | |
| Poloxamer 407 | Structuring polymer | − | ++ | − |
| Carbopol 934 | Polymer/viscosity modifier | ++ | ++ | − |
| Mannitol | Control | − | − | − |
| Sodium lactate | pH modification | ++ | ++ | ++ |
| Lactic acid | pH modification/ | ++ | ++ | ++ |
| | enhancement of drug | | | |
| | product effectiveness | | | |

++ suitable for dosage form
+ potentially suitable for dosage form
− not suitable for dosage form

### Example 12: Formulations of Dosage Forms Comprising a Substantially Complete Vaginal Microbiota Preparation

Based on excipient data obtained from the formulation work using a simulated vaginal fluid (Example 11) and the viability testing performed (data not shown), a number of formulations were generated that contained substantially complete vaginal microbiota preparations (SCVMP).

Lyophilized Gel 1:

The formulation comprised NaCMC and a substantially complete vaginal microbiota preparation and was subjected to lyophilization.

30 mg of NaCMC was weighed and transferred into a lyophilization vial. 1 SCVMP was transferred into the lyophilization vial. 1 mL lactate buffer was used to rinse and take out any remaining SCVMP from its vial and this was then transferred to the lyophilization vial. The vial was capped and swirled by hand to form a homogeneous gel that was then freeze-dried.

Lyophilized Gel 2:

The formulation comprised poloxamer and a substantially complete vaginal microbiota preparation and was subjected to lyophilization.

240 mg of Poloxamer 407 was weighed and transferred into a lyophilization vial. 1 SCVMP was transferred into the lyophilization vial. 1 mL lactate buffer was used to rinse and take out any remaining SCVMP from its vial and this was then transferred to the lyophilization vial. The vial was capped and swirled by hand to form a homogeneous gel that is then freeze-dried.

Frozen Gel 1:

The formulation comprised hyaluronic acid and a substantially complete vaginal microbiota preparation and was subjected to freezing at −80° C.

45 mg of hyaluronic acid was weighed and transferred into a lyophilization vial. 1 SCVMP was transferred into the lyophilization vial. 1 mL lactate buffer was used to rinse and take out any remaining SCVMP from its vial and this was then transferred to the lyophilization vial. The vial was capped and swirled by hand to form a homogeneous gel that was then frozen at −80° C.

Frozen Gel 2:

The formulation comprised poloxamer and a substantially complete vaginal microbiota preparation and was subjected to freezing at −80° C.

240 mg of poloxamer 407 was weighed and transferred into a lyophilization vial. 1 SCVMP was transferred into the lyophilization vial. 1 mL lactate buffer was used to rinse and take out any remaining SCVMP from its vial and this was then transferred to the lyophilization vial. The vial was capped and swirled by hand to form a homogeneous gel that was then frozen at −80° C.

Lyophilized Tablet 1:

The formulation comprised NaCMC and a substantially complete vaginal microbiota preparation and was subjected to lyophilization.

30 mg of NaCMC was weighed and transferred into a lyophilization vial. 1 SCVMP was transferred into the lyophilization vial. 1 mL lactate buffer was used to rinse and take out any remaining SCVMP from its vial and this was then transferred to the lyophilization vial. The vial was capped and swirled by hand to form a homogeneous gel that is then freeze dried. Once lyophilized, 300 mg of NaCMC were added to substance and compressed to 1 ton.

Lyophilized Tablet 2:

The formulation comprised poloxamer and a substantially complete vaginal microbiota preparation and was subjected to lyophilization.

30 mg of Poloxamer 407 was weighed and transferred into a lyophilization vial. 1 SCVMP was transferred into the lyophilization vial. 1 mL lactate buffer was used to rinse and take out any remaining SCVMP from its vial and this was then transferred to the lyophilization vial. The vial was capped and swirled by hand to form a homogeneous gel that was then freeze-dried. Once lyophilized, 300 mg of PVP (Kollidon) were added to substance and compressed to 0.5 ton.

PVA Film Disk

The formulation comprised 12% PVA and a substantially complete vaginal microbiota preparation sandwiched between two PVA layers (approximately 0.25 mL of PVA per layer).

Viable cell counts (VCC) were determined at t=0 after formulating the substantially complete vaginal microbiota preparation. Viability after 1 month and 2 months is also being determined and both VCC and CFU are assessed.

TABLE 8
Viable cell counts at t = 0 after formulation.

| | Donor #/ | CFU of | | Viable cell |
|---|---|---|---|---|
| Formulation | Visit # | donation | Species | count (VCC) |
| Lyophilized gel 1: CMC | E2 | 1.80E+08 | L. crispatus + L. iners | 5.94E+07 |
| | E6 | 8.70E+07 | L. crispatus + L. iners | 4.12E+07 |
| Lyophilized gel 2: | C4 | 4.60E+08 | L. crispatus + L. iners | 2.20E+08 |
| Poloxamer | C6 | 4.00E+08 | L. crispatus + L. iners | 1.14E+08 |
| Frozen gel 1: Hyaluronic | A1 | 7.30E+09 | L. crispatus | 7.86E+08 |
| acid | | | | |
| Frozen gel 2: Poloxamer | G8 | 1.70E+09 | L. jensenii + L. iners | |
| PVA disc | B3 | 5.10E+08 | L. crispatus | 1.95E+08 |
| Lyophilized tablet 1: CMC | D1 | 1.00E+08 | L. gasseri + L. iners | 6.28E+07 |
| | D7 | 2.20E+08 | L. gasseri + L. iners | 1.00E+07 |
| Lyophilized tablet 2: | D5 | 1.30E+08 | L. gasseri + L. iners | 4.60E+08 |
| Poloxamer/PVP | D9 | 1.30E+08 | L. gasseri + L. iners | 5.04E+08 |

Substantially complete vaginal microbiota preparations can be lyophilized and formulated into, e.g., gels and tablets as well as other dosage forms that can be filled with lyophilized products, such as, e.g., capsules. Substantially complete vaginal microbiota preparations can also be formulated into gels that can be frozen, as well as into liquid media (e.g., with glycerol) that can be frozen. Substantially complete vaginal microbiota preparations can also be formulated into (air-dried) films, that could be, e.g., shaped like disks. Losses in viability range from approximately 0.5 log to 1 log at the formulation step depending on excipient and dosage form.

### REFERENCES

Anahtar M N, Byrne E H, Doherty K E, Bowman B A, Yamamoto H S, Soumillon M, Padavattan N, Ismail N, Moodley A, Sabatini M E, Ghebremichael M S, Nusbaum C, Huttenhower C, Virgin H W, Ndung'u T, Dong K L, Walker B D, Fichorova R N, Kwon D S. 2015. Cervicovaginal bacteria are a major modulator of host inflammatory responses in the female genital tract. Immunity 42:965-976. https://doi.org/10.1016/j.immuni.2015.04.019
Lennard K, Dabee S, Barnabas S L, Havyarimana E, Blakney A, Jaumdally S Z, Botha G, Mkhize N N, Bekker L-G, Lewis D A, Gray G, Mulder N, Passmore J-AS, Jaspan H B. 2018. Microbial composition predicts genital tract inflammation and persistent bacterial vaginosis in South African adolescent females. Infect Immun 86:e00410-17. https://doi.org/10.1128/IAI.00410-17.
Masson L, Mlisana K, Little F, Werner L, Mkhize N N, Ronacher K, Gamieldien H, Williamson C, Mckinnon L R, Walzl G, Abdool Karim Q, Abdool Karim S S, Passmore J-AS. 2014. Defining genital tract cytokine signatures of sexually transmitted infections and bacterial vaginosis in women at high risk of HIV infection: a cross-sectional study. Sex Transm Infect 90:580-588. https://doi.org/10.1136/sextrans-2014-051601
Fichorova R N, Morrison C S, Chen P-L, Yamamoto H S, Govender Y, Junaid D, Ryan S, Kwok C, Chipato T, Salata R A, Doncel G F. 2020. Aberrant cervical innate immunity predicts onset of dysbiosis and sexually transmitted infections in women of reproductive age. PLoS One 15:e0224359. https://doi.org/10.1371/journal.pone.0224359.
Garrett N., Mtshali, A., Osman, F. et al. Impact of point-of-care testing and treatment of sexually transmitted infections and bacterial vaginosis on genital tract inflammatory cytokines in a cohort of young South African women. Sex Transm Infect. 2021 December; 97(8):555-565.
Camargo Campos A C, Candido Murta E F, Michelin M A, and Reis C. Evaluation of Cytokines in Endocervical Secretion and Vaginal pH from Women with Bacterial Vaginosis or Human Papillomavirus. Obstetrics and Gynecology Volume 2012, Article ID 342075.
Cherne M D, Cole A L, Newberry L, Schmidt-Owens M, Deichen M, Cole A M. Matrix Metalloproteinases Expressed in Response to Bacterial Vaginosis Disrupt the Endocervical Epithelium, Increasing Transmigration of HIV. Infect Immun. 2020 Mar. 23; 88(4):e00041-20.

Enumerated Items
1. A vaginal delivery system comprising;
   a) a dosage form suitable for administering a composition to the vaginal cavity, and
   b) a composition comprising a substantially complete vaginal microbiota preparation, wherein (i) the preparation comprises one to five bacterial species from the genus Lactobacillus, and (ii) the one to five bacterial species comprise about 80-99% of the preparation, and wherein the composition further comprises a pharmaceutically acceptable carrier or diluent.
2. The vaginal delivery system of item 1, wherein the dosage form is an applicator, dispenser or suppository.
3. The vaginal delivery system of item 1 or 2, wherein the preparation comprises about 80-99% Lactobacillus crispatus.
4. The vaginal delivery system of item 1 or 2, wherein the preparation comprises about 80-99% Lactobacillus iners.
5. The vaginal delivery system of item 1 or 2, wherein the preparation comprises about 80-99% Lactobacillus jensenii.
6. The vaginal delivery system of item 1 or 2, wherein the preparation comprises about 80-99% Lactobacillus gasseri.
7. The vaginal delivery system of item 1 or 2, wherein the preparation comprises about 80-99% of one, two, three, or four of Lactobacillus crispatus, Lactobacillus iners, Lactobacillus jensenii, and Lactobacillus gasseri.
8. The vaginal delivery system of any one of the preceding items, wherein the preparation is substantially free of pathogens and pathobionts.
9. The vaginal delivery system of item 8, wherein the pathogen or pathobiont is one or more of Gardnerella spp., Atopobium spp., Prevotella spp.
10. The vaginal delivery system of item 8, wherein the preparation comprises less than 5% of Gardnerella spp., Atopobium spp., and Prevotella spp.
11. The vaginal delivery system of any one of the preceding items, wherein the preparation is substantially free of antimicrobial resistance (AMR) genes.
12. The vaginal delivery system of any one of the preceding items, wherein the preparation is substantially free of human sperm (spermatozoa).
13. The vaginal delivery system of any one of the preceding items, wherein the composition further comprises a spermicide.
14. The vaginal delivery system of any one of the preceding items, wherein the composition further comprises an acidifying agent.
15. The vaginal delivery system of any one of the preceding items, wherein the composition further comprises lactic acid.
16. The vaginal delivery system of any one of the preceding items, wherein the preparation comprises a glycan.
17. The vaginal delivery system of item 16, wherein said glycan is mucus.
18. The vaginal delivery system of any one of the preceding items, wherein the preparation comprises less than 20 species other than from the genus Lactobacillus.
19. The vaginal delivery system of any one of the preceding items, wherein the composition has a pH of equal or less than 4.5.
20. The vaginal delivery system of any one of the preceding items, wherein the dosage form comprises 10² to 10¹⁰ CFU of lactobacilli.
21. The vaginal delivery system of any one of the preceding items, wherein the composition has been dispensed or formulated in the dosage form.
22. The vaginal delivery system of any one of the preceding items, wherein the dosage form is sterile packaged.
23. The vaginal delivery system of item 2, wherein the applicator or dispenser comprises an open end (e.g., tip) for insertion into the vaginal cavity, and a dispensing end (e.g., a plunger or piston) to expel the composition.
24. The vaginal delivery system of any one of the preceding items, wherein the preparation is obtained by a culture-independent method.
25. The vaginal delivery system of any one of the preceding items, wherein the dosage form is calibrated (e.g., has a volume indication or maximal volume indication or control).
26. The vaginal delivery system of any one of the preceding items, wherein the dosage form is a single use or multiple use device.
27. The vaginal delivery system of any one of the preceding items, wherein the dosage form is designed from inert polymeric materials, such as polystyrene or polypropylene.
28. A pharmaceutical composition formulated for vaginal administration comprising a substantially complete vaginal microbiota preparation, wherein (i) the preparation comprises one to five bacterial species from the genus Lactobacillus, and (ii) the one to five bacterial species comprise about 80-99% of the preparation, and wherein the composition further comprises a pharmaceutically acceptable carrier or diluent.
29. The pharmaceutical composition of item 28, wherein the preparation comprises 80-99% Lactobacillus crispatus.
30. The pharmaceutical composition of item 28, wherein the preparation comprises 80-99% Lactobacillus iners.
31. The pharmaceutical composition of item 28, wherein the preparation comprises about 80-99% Lactobacillus jensenii.
32. The pharmaceutical composition of item 28, wherein the preparation comprises about 80-99% Lactobacillus gasseri.
33. The pharmaceutical composition of item 28, wherein the preparation comprises about 80-99% of one, two, three, or four of Lactobacillus crispatus, Lactobacillus iners, Lactobacillus jensenii, and Lactobacillus gasseri.
34. The pharmaceutical composition of any one of the preceding items, wherein the preparation is substantially free of pathogens and pathobionts.
35. The pharmaceutical composition of item 34, wherein the pathogen or pathobiont is one or more of Gardnerella spp., Atopobium spp., and Prevotella spp.
36. The pharmaceutical composition of item 34, wherein the preparation comprises less than 5% of Gardnerella spp., Atopobium spp., and Prevotella spp.
37. The pharmaceutical composition of any one of the preceding items, wherein the preparation is substantially free of antimicrobial resistance (AMR) genes.
38. The pharmaceutical composition of any one of the preceding items, wherein the preparation is substantially free of human sperm (spermatozoa).
39. The pharmaceutical composition of any one of the preceding items, wherein the composition further comprises a spermicide.
40. The pharmaceutical composition of any one of the preceding items, wherein the composition further comprises an acidifying agent.
41. The pharmaceutical composition of any one of the preceding items, wherein the composition further comprises lactic acid.
42. The pharmaceutical composition of any one of the preceding items, wherein the preparation comprises a glycan.
43. The pharmaceutical composition of item 42, wherein said glycan is mucus.
44. The pharmaceutical composition of any one of the preceding items, wherein the preparation comprises less than 20 species other than from the genus Lactobacillus.
45. The pharmaceutical composition of any one of the preceding items, wherein the composition has a pH of equal or less than 4.5.
46. The pharmaceutical composition of any one of the preceding items, wherein the preparation is obtained by a culture-independent method.
47. A method for vaginal administration to a human female subject in need thereof, of a composition comprising a substantially complete vaginal microbiota preparation using a device, wherein the device comprises an open end (e.g., a tip) for insertion into the vaginal cavity, and a dispensing end (e.g., a plunger or piston) to expel the composition through the open end, the method comprising:
   a) introducing the open end into a vaginal cavity,
   b) expelling the composition into the vaginal cavity,
thereby administering the composition to the vaginal cavity.
48. The method of item 47, wherein the device is an applicator or dispenser of any one of items 2-27.
49. The method of item 47, wherein the composition is a pharmaceutical composition of any one of items 28-46.
50. The method of any of the preceding items, wherein expelling the composition comprises contacting a mucosal or endometrial surface of the vagina.
51. The method of any one of items 47-50, wherein the subject is in need of improving her vaginal health.
52. The method of any one of items 47-51, wherein administration to the subject is carried out while the subject is in a lithotomy position.
53. The method of any one of items 47-52, wherein the composition is of a suitable viscosity that allows the majority of it to stay in the vaginal cavity for at least 5, 10, 20, or 30 minutes, when the subject is in upright position.
54. The method of any one of items 47-53, wherein the device is calibrated (e.g., has a volume indication or maximal volume indication or control).
55. The method of any one of items 47-54, wherein the device administers about 10² to 10¹⁰ CFU of lactobacilli per administration.
56. The method of any one of items 47-55, wherein administering is performed one, two, or three times within 3-21 days.
57. The method of any one of items 47-55, wherein administering is performed once every one to four weeks for a period of one to six months.
58. The method of any one of items 47-57, wherein the device is a single use or multiple use device.
59. The method of any of the preceding items, wherein the method further comprises colonizing the vaginal mucosa with one or more Lactobacillus species.
60. The method of item 59, wherein one or more Lactobacillus species are one, two, three, or four of Lactobacillus crispatus, Lactobacillus iners, Lactobacillus jensenii, and Lactobacillus gasseri.
61. The method of item 59 or 60, wherein the preparation comprises about 80-99% Lactobacillus crispatus.
62. The method of item 59 or 60, wherein the preparation comprises about 80-99% Lactobacillus iners.
63. The method of item 59 or 60, wherein the preparation comprises about 80-99% Lactobacillus jensenii.
64. The method of item 59 or 60, wherein the preparation comprises about 80-99% Lactobacillus gasseri.
65. The method of item 59 or 60, wherein the preparation comprises about 80-99% of one, two, three, or four of Lactobacillus crispatus, Lactobacillus iners, Lactobacillus jensenii, and Lactobacillus gasseri.
66. The method of any one of items 47-65, wherein the preparation is substantially free of pathogens and pathobionts.
67. The method of item 66, wherein the pathogen or pathobiont is one or more of Gardnerella spp., Atopobium spp., and Prevotella spp.
68. The method of item 66, wherein the preparation comprises less than 5% of Gardnerella spp., Atopobium spp., and Prevotella spp.
69. The method of any one of items 47-68, wherein the preparation is substantially free of antimicrobial resistance (AMR) genes.
70. The method of any one of items 47-69, wherein the preparation is substantially free of human sperm (spermatozoa).
71. The method of any one items 47-70, wherein the composition further comprises a spermicide.
72. The method of any one of items 47-71, wherein the composition further comprises an acidifying agent.
73. The method of any one of items 47-72, wherein the composition further comprises lactic acid.
74. The method of any one of items 47-73, wherein the preparation comprises a glycan.
75. The method of item 74, wherein said glycan is mucus.
76. The method of any one items 47-75, wherein the preparation comprises less than 20 species other than from the genus Lactobacillus.
77. The method of any one of items 47-76, wherein the composition has a pH of equal or less than 4.5.
78. The method of any one of items 47-77, wherein the preparation is obtained by a culture-independent method.
79. A kit comprising the dosage form (a) and composition (b) of any one of items 1-46.
80. The kit of item 79 wherein the dosage form (a) and composition (b) are in separate sterile packages.
81. The kit of item 79 or 80, wherein the dosage form is a multi-dosage form.
82. The kit of any one of items 79-81, wherein the dosage form (a) and/or the composition (b) comprises multiple doses.
83. The kit of any one of items 79-82, wherein the dosage form is a single use or
84. The kit of any one of items 79-83, wherein the composition is freeze-dried or lyophilized.
85. The kit of item 84, wherein the kit further comprises a resuspension medium for the freeze-dried or lyophilized composition.
86. A substantially complete vaginal microbiota preparation comprising one to five bacterial species from the genus Lactobacillus derived from a female donor's genitourinary tract (e.g., vaginal microbial niche) secretion, wherein the one to five bacterial species comprise about 80-99% of the preparation and one or more species of said bacterial species are capable of engrafting (e.g., colonizing) in a female recipient's genitourinary tract.
87. The preparation of item 86, wherein the preparation comprises about 80-99% Lactobacillus crispatus.
88. The preparation of item 86, wherein the preparation comprises about 80-99% Lactobacillus iners.
89. The preparation of item 86, wherein the preparation comprises about 80-99% Lactobacillus jensenii.
90. The preparation of item 86, wherein the preparation comprises about 80-99% Lactobacillus gasseri.
91. The preparation of item 86, wherein the preparation comprises about 80-99% of one, two, three, or four of Lactobacillus crispatus, Lactobacillus iners, Lactobacillus jensenii, and Lactobacillus gasseri.
92. The preparation of item 86, formulated as a pharmaceutical composition (e.g., a pharmaceutical composition of any one of items 28-47).
93. The pharmaceutical composition of any one of the preceding items, wherein said pharmaceutical composition further comprises an active agent selected from the group consisting of an antibiotic, immunological agent, and a hormonal agent.
94. The pharmaceutical composition of any one of the preceding items, wherein said pharmaceutical composition is in the form of a suspension, spray, gel, cream, powder, capsule, solution for lavages, ovules, a vaginal insert, tampon, tablets or a microencapsulated product.
95. A method of producing substantially complete vaginal microbiota preparation of any one of the preceding items, comprising:
   A. providing a microbiota sample from a donor female genitourinary tract; wherein step A comprises optionally (1), (2), (3), (4), (5) or any combination thereof:
      (1) adding a diluent to the microbiota sample to create a diluted sample,
      (2) removing a portion of the diluted microbiota sample for testing (e.g., nucleic acid sequencing),
      (3) pre-cooling for either refrigeration or freezing of the remainder of the microbiota sample,
      (4) storing the refrigerated or frozen microbiota sample under quarantine,
      (5) holding the refrigerated or frozen microbiota sample under quarantine until any completion of any combination of (a) testing the donor to exclude the substantial presence of one or more transmissible pathogens (e.g., blood, and/or cervicovaginal secretions, and/or urine sample testing), (b) confirming the composition and viability of the microbiota, or (c) further confirming the health of the female donor by a plurality of post-screening tests occurring within a time period of 30-90 days post-donation; and
   B. releasing the refrigerated or frozen microbiota sample from quarantine to define substantially complete vaginal microbiota preparation.
96. The method of item 95, wherein the microbiota sample from the donor female is a cervicovaginal secretion.
97. The method of item 95 or 96, wherein the microbiota sample comprises a glycan.
98. The method of item 97, wherein said glycan is mucus.
99. The method of any one of items 95-98, wherein the microbiota sample comprises at least 150 mg.
100. The method of any one of items 95-99, wherein the microbiota sample comprises about 80-99% Lactobacillus crispatus.
101. The method of any one of items 95-99, wherein the microbiota sample comprises about 80-99% Lactobacillus iners.
102. The method of any one of items 95-99, wherein the microbiota sample comprises about 80-99% Lactobacillus jensenii.
103. The method of any one of items 95-99, wherein the microbiota sample comprises about 80-99% Lactobacillus gasseri.
104. The method of any one of items 95-103, wherein the microbiota sample comprises about 80-99% of one, two, three, or four of Lactobacillus crispatus, Lactobacillus iners, Lactobacillus jensenii, and Lactobacillus gasseri.
105. The method of any one of items 95-104, wherein step B of item 95 comprises determining that the microbiota sample is substantially free of pathogens and pathobionts.
106. The method of any one of items 95-105, wherein step B of item 95 comprises determining that the microbiota sample is substantially free of Gardnerella spp., Atopobium spp., and Prevotella spp.
107. The method of any one of items 95-106, wherein step B of item 95 comprises determining that the microbiota sample is substantially free of antimicrobial resistance (AMR) genes.
108. The method of any one of items 95-107, wherein step B of item 95 comprises determining that the microbiota sample is substantially free of human sperm (spermatozoa).
109. The method of any one of items 95-108, wherein step B of item 95 comprises determining that the female donor is substantially free of any one or more (two or more, three or more, or four or more) of: (i) bacteria involved in bacterial vaginosis (e.g., Gardnerella spp. and Prevotella spp.), (ii) yeast (e.g., Candida, Cryptococcus, and Saccharomyces species), (iii) sexually transmitted pathogens (e.g., Neisseria gonorrhea, Chlamydia trachomatis, and Trichomonas vaginalis), (iv) bacteria involved in urinary tract infections (e.g., E. coli, Staphylococcus, Chlamydia, and Mycoplasma), and (v) viruses (e.g., HIV, human papilloma virus (HPV), hepatitis B virus, hepatitis C virus, HSV-2), or any combination thereof.
110. A method for restoring a healthy human vaginal microbiota balance in the female genitourinary tract of a human subject comprising administering to a subject in need of such restoration an effective amount of a pharmaceutical composition comprising a substantially complete vaginal microbiota preparation of any one of items 28 to 46, optionally using a vaginal delivery system of any one of items 1 to 27, thereby restoring a healthy vaginal microbiota balance.
111. The method of item 110, wherein the female subject exhibits a dysbiotic microbiota in the genitourinary tract and restoring a healthy vaginal microbiota balance provides treatment of the dysbiosis.
112. The method of item 110 or 111, wherein the female subject is treated with an effective amount of an antimicrobial agent (e.g., an antibiotic) to substantially diminish the residing dysbiotic microbiota prior to administration of the pharmaceutical composition comprising a substantially complete vaginal microbiota preparation.
113. The method of any one of items 110 to 112, wherein restoring a healthy vaginal microbiota balance comprises colonization and engraftment of one or more species provided by the pharmaceutical composition comprising a substantially complete vaginal microbiota preparation.
114. The method of any one of items 110 to 113, wherein restoring a healthy vaginal microbiota balance comprises one or more of: (a) a reduction in the relative abundance of pathogen and/or pathobiont residing in the genitourinary tract (e.g., less than 10% relative abundance of pathogen and/or pathobiont), (b) an increase in relative abundance of Lactobacillus above 50% (above 60%, or above 70%), (c) a reduction in one or more pro-inflammatory markers (e.g., local or systemic), and/or (d) a lowering of vaginal pH (e.g., by at least pH 0.3, 0.5, 1.0, or 1.5), and/or (e) a change in grade based on the Ison-Hay scoring system (e.g., grade 0 or grade I), when compared to the values of (a), (b), (c), (d) or (e) determined in the same subject prior to administration of the pharmaceutical composition comprising a substantially complete vaginal microbiota preparation.
115. The method of any one of items 110 to 114, wherein restoring a healthy vaginal microbiota balance comprises engraftment of a microbial community dominated (e.g., at least 50%, 60%, 70% or at least 80%) by one or more species of lactic acid producing bacteria.
116. The method of item 115, wherein the one or more species of lactic acid producing bacteria is selected from: one, two, three, or four of Lactobacillus crispatus, Lactobacillus iners, Lactobacillus jensenii, and Lactobacillus gasseri.
117. The method of any one of items 110 to 116, wherein restoring a healthy vaginal microbiota balance comprises achieving a pH of the vaginal tract of between about pH 3.5 to 4.5.
118. The method of items 115 or 116, wherein the microbial community remains dominated by lactic acid producing bacteria (e.g., lactobacilli) for at least 1 week, or at least 2, 3, 4 weeks.
119. The method of items 115 or 116, wherein the microbial community remains dominated by lactic acid producing bacteria (e.g., lactobacilli) for at least 2, 3, 4, 5 or at least 6 months.
120. The method of any one of items 110 to 119, further comprising administering one or more additional active agents, selected from the group consisting of an antibiotic, immunological agent, and a hormonal agent.
121. The method of any one of items 110 to 120, wherein the subject is Caucasian.
122. The method of any one of items 110 to 120, wherein the subject is Asian.
123. The method of any one of items 110 to 120, wherein the subject is Black/African.
124. The method of any one of items 110 to 120, wherein the subject is Hispanic.
125. The method of any one of items 110 to 124, wherein the subject is of child-bearing age.
126. The method of any one of items 110 to 124, wherein the subject is post-menopausal.
127. The method of any one of items 110 to 126, wherein restoring a healthy vaginal microbiota balance provides an antipathogenic microbial niche/vaginal environment.
128. The method of any one of items 110 to 127, wherein restoring a healthy vaginal microbiota balance provides an anti-inflammatory or low-inflammatory microbial niche/vaginal environment.
129. A method of treating dysbiosis in the female genitourinary tract of a human subject comprising administering to a subject in need of such treatment an effective amount of a pharmaceutical composition comprising a substantially complete vaginal microbiota preparation of any one of items 28 to 46, optionally using a vaginal delivery system of any one of items 1 to 27, thereby treating the dysbiosis.
130. The method of item 129, wherein the dysbiosis is associated with an infection of the genitourinary tract.
131. The method of item 129 or 130, wherein the infection is one or more of (recurrent) bacterial vaginosis (BV), vaginal candidiasis and trichomoniasis.
132. A method of treating (chronic) inflammation in the female genitourinary tract of a human subject comprising administering to a subject in need of such treatment an effective amount of a pharmaceutical composition comprising a substantially complete vaginal microbiota preparation of any one of items 28 to 46, optionally using a vaginal delivery system of any one of items 1 to 27, thereby treating the inflammation.
133. A method of treating (recurrent) bacterial vaginosis (BV) in the female genitourinary tract of a human subject comprising administering to a subject in need of such treatment an effective amount of a pharmaceutical composition comprising a substantially complete vaginal microbiota preparation of any one of items 28 to 46, optionally using a vaginal delivery system of any one of items 1 to 27, thereby treating (recurrent) BV.
134. A method of treating vaginal candidiasis in the female genitourinary tract of a human subject comprising administering to a subject in need of such treatment an effective amount of a pharmaceutical composition comprising a substantially complete vaginal microbiota preparation of any one of items 28 to 46, optionally using a vaginal delivery system of any one of items 1 to 27, thereby treating vaginal candidiasis.
135. A method of treating trichomoniasis in the female genitourinary tract of a human subject comprising administering to a subject in need of such treatment an effective amount of a pharmaceutical composition comprising a substantially complete vaginal microbiota preparation of any one of items 28 to 46, optionally using a vaginal delivery system of any one of items 1 to 27, thereby treating trichomoniasis.
136. The method of any one of items 129 to 135, further comprising administering one or more additional active agents, selected from the group consisting of an antibiotic, immunological agent and a hormonal agent.

1. A pharmaceutical composition for use in treating inflammation in the female genitourinary tract of a human subject, wherein the female subject exhibits a dysbiotic microbiota in the genitourinary tract, said method comprising administering to the subject an effective amount of the pharmaceutical composition, wherein the pharmaceutical composition comprises a substantially complete vaginal microbiota preparation, wherein the preparation
(i) comprises one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the preparation; and
(ii) comprises less than 5% of Gardnerella spp., Atopobium spp., and Prevotella spp.;
wherein the pharmaceutical composition comprises a pharmaceutically acceptable carrier or diluent.

2. The pharmaceutical composition for use according to claim 1, wherein the female subject is not treated with an antibiotic prior to or during treatment of the inflammation.

3. The pharmaceutical composition for use according to claim 1, wherein the pharmaceutical composition is administered to the vaginal cavity of the subject in need thereof.

4. The pharmaceutical composition for use according to claim 3, wherein prior to administering the pharmaceutical composition, a vaginal wash is performed and/or wherein the vaginal wash is subsequently rinsed with lactic acid.

5. The pharmaceutical composition for use according to 4, wherein the vaginal wash is performed with saline, lactic acid or an antiseptic agent, optionally wherein the antiseptic agent is povidone-iodine or chlorohexidine.

6. The pharmaceutical composition for use according to claim 1, wherein the subject has not been diagnosed with bacterial vaginosis.

7. The pharmaceutical composition for use according to claim 1, wherein the substantially complete vaginal microbiota preparation further comprises vaginal transudate and/or mucus, optionally wherein the mucus is cervicovaginal mucus.

8-10. (canceled)

11. The pharmaceutical composition for use according to claim 1, wherein about 80-99.9% of all detectable bacterial species of the preparation consist of Lactobacillus crispatus.

12. The pharmaceutical composition for use according to claim 1, wherein about 80-99.9% of all detectable bacterial species of the preparation consist of
(a) Lactobacillus crispatus;
(b) Lactobacillus iners;
(c) Lactobacillus crispatus and Lactobacillus iners;
(d) Lactobacillus crispatus and Lactobacillus jensenii;
(e) Lactobacillus crispatus, Lactobacillus iners and Lactobacillus jensenii;
(f) Lactobacillus crispatus, Lactobacillus gasseri and Lactobacillus jensenii;
(g) Lactobacillus iners and Lactobacillus jensenii;
(h) Lactobacillus iners, Lactobacillus jensenii, and Lactobacillus gasseri;
(i) Lactobacillus iners and Lactobacillus gasseri; or
(j) Lactobacillus crispatus, Lactobacillus iners, Lactobacillus jensenii, and Lactobacillus gasseri.

13. The pharmaceutical composition for use according to claim 12, wherein for (c) to (j) Lactobacillus crispatus, Lactobacillus iners, Lactobacillus jensenii, Lactobacillus gasseri may be present in greater relative quantity than one or more of the other vaginal lactobacilli present in the preparation.

14. (canceled)

15. The pharmaceutical composition for use according to claim 1, wherein less than 10% of all detectable bacterial species of the dysbiotic vaginal microbiota in the genitourinary tract of the female subject belong to the genus Lactobacillus, and at least 20%, 30%, 40%, 50%, 60%, 70% or more of all detectable bacterial species of the dysbiotic vaginal microbiota are pathogens or pathobionts selected from the group consisting of Gardnerella spp., Atopobium spp., and Prevotella spp.

16-17. (canceled)

18. A substantially complete vaginal microbiota preparation or pharmaceutical composition comprising the same, wherein said preparation
(i) comprises one, two, three or four bacterial species from the genus Lactobacillus, selected from Lactobacillus crispatus, Lactobacillus iners, Lactobacillus jensenii, Lactobacillus gasseri, which comprise about 80-99.9% of all detectable bacterial species of the preparation; and
(ii) comprises less than 5% of Gardnerella spp., Atopobium spp., and Prevotella spp.;
wherein the preparation and/or the pharmaceutical composition comprises a pharmaceutically acceptable carrier or diluent.

19-21. (canceled)

22. The preparation or pharmaceutical composition of claim 18 one of claims 18 to 21, said preparation or pharmaceutical composition comprising further lactobacilli other than Lactobacillus crispatus, Lactobacillus iners, Lactobacillus jensenii, or Lactobacillus gasseri, wherein the further lactobacilli are present in a concentration of about 0.01-1% of all detectable bacterial species of the preparation.

23. (canceled)

24. A dosage form comprising the pharmaceutical composition or the substantially complete vaginal microbiota preparation of claim 18 formulated for vaginal administration, wherein the dosage form is solid, semi-solid, a liquid formulation, or a film-forming formulation.

25-28. (canceled)

29. A vaginal delivery system for administration to the vaginal cavity comprising the pharmaceutical composition or the substantially complete vaginal microbiota preparation of claim 18, in a dosage form that is suitable for an applicator, dispenser or suppository.

30-32. (canceled)

33. A method for vaginal administration of the pharmaceutical composition or the substantially complete vaginal microbiota preparation of claim 18 to a human female subject, wherein the composition or preparation is administered using a device, wherein the device comprises an open end (e.g., a tip) for insertion into the vaginal cavity, and a dispensing end (e.g., a plunger or piston) to expel the composition or preparation through the open end, the method comprising:
a) introducing the open end into a vaginal cavity,
b) expelling the composition or preparation into the vaginal cavity,
thereby administering the composition or preparation to the vaginal cavity.

34-39. (canceled)

40. A method of producing a substantially complete vaginal microbiota preparation, said method comprising
A. providing a microbiota sample from a donor female genitourinary tract; wherein step A comprises one, two, or three of steps (1), (2), (3) or any combination thereof, and both steps (4) and (5):
(1) adding a diluent to the microbiota sample to create a diluted sample,
(2) removing a portion of the diluted microbiota sample for testing (e.g., nucleic acid sequencing),
(3) pre-cooling for either refrigeration or freezing of the remainder of the microbiota sample,
(4) storing the refrigerated or frozen microbiota sample under quarantine,
(5) holding the refrigerated or frozen microbiota sample under quarantine until any completion of any combination of (a) testing the donor to exclude the substantial presence of one or more transmissible pathogens (e.g., blood, and/or cervicovaginal secretions, and/or urine sample testing), (b) confirming the composition and viability of the microbiota, or (c) further confirming the health of the female donor by a plurality of post-screening tests occurring within a time period of 30-90 days post-donation; and
B. releasing the refrigerated or frozen microbiota sample from quarantine to define the substantially complete vaginal microbiota preparation.

41-60. (canceled)
